(19) United States
(12) Patent Application Publication
(10) Pub. No.: US 2023/0364219 A1
(43) Pub. Date: Nov. 16, 2023

(54) SARS COV-2 SPIKE PROTEIN CONSTRUCT

(71) Applicant: GlaxoSmithKline Biologicals SA, Rixensart (BE)

(72) Inventors: Dong YU, Rockville, MD (US); Giulietta MARUGGI, Rockville, MD (US); Jason W. WESTERBECK, Rockville, MD (US); Jeffrey B. ULMER, Rockville, MD (US); Jason P. LALIBERTE, Rockville, MD (US); Kate LUISI, Collegeville, PA (US); Lin QU, Collegeville, PA (US)

(73) Assignee: GlaxoSmithKline Biologicals SA, Rixensart (BE)

(21) Appl. No.: 17/918,676

(22) PCT Filed: Apr. 16, 2021
(86) PCT No.: PCT/IB2021/053161

§ 371 Date: Oct. 13, 2022

Related U.S. Application Data
(60) Provisional application No. 63/029,813, filed on May 26, 2020.
(60) Provisional application No. 63/010,763, filed on Apr. 16, 2020.

(51) Int. Cl. A61K 39/215; A61P 37/04

(52) U.S. Cl. CPC A61K 39/215; A61P 37/04; A61K 2039/55566

(57) ABSTRACT

Compounds useful as components of immunogenic compositions for the induction of an immunogenic response in a subject against infection, methods for their use in treatment, and processes for their manufacture are provided herein. The compounds comprise a nucleic acid construct comprising a sequence which encodes an antigen, in particular a Coronavirus antigen.

This application claims priority to U.S. Provisional Pat. Application 63/010763, filed Apr. 16, 2020 and U.S. Provisional Pat. Application 63/029813, filed May 26, 2020, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 31, 2021, is named VU66912_SL.txt and is 1,216,512 bytes in size.

### FIELD OF THE INVENTION

This invention is in the field of treating and preventing infections such as viral infections. In particular, the present invention relates to the use of Coronaviral antigens for treating and preventing Coronavirus infections.

### BACKGROUND TO THE INVENTION

The sequences of the Coronavirus spike protein (S) antigen for various Coronavirus strains are known. A prefusion stabilized Coronavirus S antigen was published in WO2018081318. Coronavirus sequences associated with COVID19 were published in 2020, see GenBank accession number MN908947. A prefusion stabilized Coronavirus S antigen ectodomain was published in Wrapp et al. (2020) “Cryo-EM structure of the 2019-nCoV spike in the prefusion conformation.” Science, 367:1260-1263.

Given the concerning disease burden and the potential for rapid dissemination, there is an urgent need for the development of components for use in a Coronavirus vaccine.

In the context of a vaccine, and in particular a Coronavirus vaccine, based on the use of a nucleic acid construct to express an antigen in target cells, there is a need to increase to increase expression and presentation of the antigen to the immune system.

### SUMMARY OF THE INVENTION

The present inventors provide constructs in which the expression and secretion or cell-surface expression of an antigen is enhanced. In some embodiments, the construct encodes a viral antigen, in particular a coronavirus antigen, wherein the expression and secretion or cell-surface expression is enhanced. Such constructs are useful as components of immunogenic compositions for the induction of an immune response in a subject. In some embodiments, such constructs are useful as components of immunogenic compositions for the induction of an immune response against Coronaviral infection. Methods for their use in treatment, and processes for their manufacture are also provided.

In one aspect, the invention provides a construct comprising a nucleic acid sequence encoding an antigen, wherein the antigen comprises a heterologous signal sequence.

In one aspect, the invention provides a construct comprising a nucleic acid sequence encoding an antigen, wherein the antigen comprises a heterologous signal sequence selected from a Gaussian Luciferase signal sequence as shown in SEQ ID NO:2, a human CD5 signal sequence as shown in SEQ ID NO:3, a human CD33 signal sequence as shown in SEQ ID NO:4, a human IL2 signal sequence as shown in SEQ ID NO:5, a human IgE signal sequence as shown in SEQ ID NO:6, a human Light Chain Kappa signal sequence as shown in SEQ ID NO:7, a JEV short signal sequence as shown in SEQ ID NO:8, a JEV long signal sequence as shown in SEQ ID NO:9, a Mouse Light Chain Kappa signal sequence as shown in SEQ ID NO:10, a SSP signal sequence as shown in SEQ ID NO:11, a Gaussian Luciferase (AKP) signal sequence as shown in SEQ ID NO:12, and variants thereof.

In one aspect, the invention provides a construct comprising a nucleic acid sequence encoding an antigen, suitably a coronavirus antigen, wherein the antigen comprises a heterologous signal sequence, a mutation with respect to the wild-type sequence which affects retention of the antigen in the endoplasmic reticulum, or a combination thereof.

In one aspect of the construct, the invention provides a self-replicating RNA comprising the construct of the invention.

In one aspect, the invention provides a DNA molecule encoding the self-replicating RNA of the invention.

In one aspect, the invention provides a composition comprising an immunologically effective amount of one or more of the constructs, self-replicating RNA or DNA of the invention.

In one aspect, the invention provides a process for producing an RNA-based vaccine comprising a step of transcribing the DNA molecule of the invention to produce a self-replicating RNA comprising a coding region for the coronavirus S protein.

In one aspect, the invention provides a composition produced by the process of the invention.

In one aspect, the invention provides a method of inducing an immune response against a Coronavirus infection in a subject in need thereof, which comprises administering to said subject an immunologically effective amount of the construct, the self-replicating RNA, the DNA molecule, or the composition of the invention.

In one aspect, the invention provides the construct, the self-replicating RNA, the DNA molecule, or the composition of the invention, for use in therapy.

In one aspect, the invention provides the construct, the RNA, the DNA molecule, or the composition of the invention, for use in preventing or treating a coronavirus infection in a subject.

In one aspect, the invention provides the construct, the self-replicating RNA, the DNA molecule, or the composition of the invention, for use in preventing or treating a SARS CoV-2 infection in a subject.

In one aspect, the invention provides the use of the construct, the self-replicating RNA, the DNA molecule, or the composition of the invention for inducing an immune response to a Coronavirus infection in a subject.

In one aspect, the invention provides the use of the construct, the self-replicating RNA, the DNA molecule, or the composition of the invention in the manufacture of a medicament inducing an immune response against a Coronavirus infection in a subject.

In one aspect, the invention provides the use of the construct, the self-replicating RNA, the DNA molecule, or the composition of the invention for inducing an immune response to a SARS CoV-2 infection in a subject.

In one aspect, the invention provides the use of the construct, the self-replicating RNA, the DNA molecule, or the composition of the invention in the manufacture of a medicament inducing an immune response against a SARS CoV-2 infection in a subject.

A. The present inventors also provide constructs encoding a coronavirus antigen. In some embodiments, a construct comprising a nucleic acid sequence selected from (a) a nucleic acid sequence encoding a polypeptide comprising the amino acid sequence of SEQ ID NO:49 and SEQ ID NO:26; (b) a nucleic acid sequence comprising the DNA sequence of SEQ ID NO:97 and SEQ ID NO:74; (c) a nucleic acid sequence comprising the RNA sequence of SEQ ID NO:145 and SEQ ID NO:122; and (d) variants or fragments of (a)-(c) are provided. In some embodiments, a self-replicating RNA molecule comprising a construct encoding a polypeptide comprising a Coronavirus spike (S) antigen, or an immunogenic variant or fragment are provided.

In some embodiments, a composition comprising an immunologically effective amount of one or more of the constructs or the self-replicating RNA molecules described beginning in section A above is provided.

In some embodiments, a method is provided for inducing an immune response against a Coronavirus infection in a subject in need thereof, which comprises administering to said subject an immunologically effective amount of a composition comprising one or more of the constructs or self-replicating RNA molecules as described beginning in section A above.

In some embodiments, a process is provided for producing an RNA-based vaccine comprising a step of transcribing a DNA molecule encoding a self-replicating RNA molecule as described beginning in section A above.

In some embodiments, a composition produced by the process of described beginning in section A above is provided.

In some embodiments, a use of the constructs, self-replicating RNA molecules, or compositions described beginning in section A above for inducing an immune response against a Coronavirus infection in a subject is provided.

In some embodiments, a use of the constructs, self-replicating RNA molecules, or compositions described beginning in section A above in the manufacture of a medicament for inducing an immune response against a Coronavirus infection in a subject is provided.

In some embodiments, a use of the constructs, self-replicating RNA molecules, or compositions described beginning in section A above for inducing an immune response against a SARS CoV-2 infection in a subject is provided.

In some embodiments, a use of the constructs, self-replicating RNA molecules, or compositions described beginning in section A above in the manufacture of a medicament for inducing an immune response against a SARS CoV-2 infection in a subject is provided.

### DESCRIPTION OF DRAWINGS/FIGURES

FIG. 1 Illustrates the vectors and sequences used to generate three SARS CoV-2 The self-amplifying mRNA (SAM) replicons. FIG. 1 discloses “GSAS” as SEQ ID NO: 190.

FIG. 2A illustrates pDNA in situ of Spike_ECTO-2P SAM replicon with furin cleavage site mutation and 2X proline mutation, pJW18; FIG. 2B illustrates pDNA in situ of Spike_WT SAM replicon (pJW20). FIG. 2C illustrates pDNA in situ of Spike_FL-2P SAM (pJW19).

FIG. 3 illustrates pDNA SAM CoV-2 Spike vectors digested with Apa1 and Pmel restriction enzymes to verify size of the spike sequence insert (3.8 Kb). Vector bands were compared to NEB 1 Kb extend DNA ladder (Ladder) and to the pDNA SAM source vector (pJL209).

FIG. 4. illustrates pDNA SAM CoV-2 Spike vectors digested with BspQI restriction enzyme prior to IVT.

FIG. 5 illustrates SAM RNA gel analysis.

FIG. 6A illustrates percent of cells containing dsRNA after electroporation as determined by J2 mouse monoclonal antibody staining. FIG. 6B illustrates geometric mean fluorescence intensity (MFI) of J2 positive cells (correlate of dsRNA amount). FIG. 6C illustrates percent of cells containing SARS CoV-2 spike protein as detected by a mouse monoclonal antibody to the S2 fragment of the spike protein from Genetex (GTX632604). FIG. 6D illustrates MFI of spike positive cells (correlate of amount of spike protein present).

FIG. 7A illustrates western blot analysis of SARS CoV-2 Spike SAM RNA replicons from BHK cells: 5% lysate from a 1 µg RNA electroporation into 1 million BHK cells. FIG. 7B illustrates western blot analysis of SARS CoV-2 Spike SAM RNA replicons from BHK cells: 5% lysate from a 1 µg RNA electroporation into 1 million BHK cells.

FIG. 8A illustrates total protein evaluation and comparison in fixed cells between percent positive spike cells and percent positive mScarlet signal from a control SAM replicon. Surface evaluation in live cells of the percent surface spike (FIG. 8B), and percent surface hACE2 binding cells (normalized by subtracting BFA+ value for each sample) (FIG. 8C). MFI values of spike or hACE2 for spike total protein (FIG. 8D), spike surface protein (FIG. 8E) and hACE2 bound to live cells (FIG. 8F).

FIG. 9A, FIG. 9B illustrates percent of cells containing dsRNA after electroporation as determined by J2 mouse monoclonal antibody staining for SAM GFP control and spike replicons. FIG. 9C depicts geometric mean fluorescence intensity (MFI) of J2 positive cells (correlate of dsRNA amount). FIG. 9D, FIG. 9E illustrate percent of cells containing mScarlet or SARS CoV-2 spike protein as detected by a mouse monoclonal antibody to the S2 fragment of the spike protein from Genetex (GTX632604). FIG. 9F, FIG. 9G depict surface evaluation in live cells of the percent surface spike, and percent surface hACE2 binding cells, respectively.

FIG. 10A, FIG. 10B illustrates 5% lysate from a 1 µg RNA electroporation into 1 million muscle cells were evaluated with and without PNGase (N-glycosidase treatment). FIG. 10C illustrates 25 µl of 10X concentrated supernatant was run per well of an 4-12% SDS-PAGE gel, and transferred to a nitrocellulose membrane.

FIG. 11A illustrates an EC50 curve of Spike_ECTO-2P SAM (JW18) (LPN) and Spike_FL-2P SAM(JW19) (LNP). FIG. 11B depicts an EC50 bar graph SAM-CoV-2 RV39 Potency protein expression assay. FIG. 11C depicts result images from HCI 10x objective: JW18: Spike_ECTO-2P SAM (LNP); JW19: Spike_FL-2P SAM (LNP).

FIG. 12 illustrates SEQ ID NO:49 depicts the polypeptide sequence of the full-length S protein, including the PP and GSAS (SEQ ID NO:190) substitutions (underlined, bold) but lacking the foldon, HRV3C protease cleavage, TwinStrepTab, and 8XHisTag (SEQ ID NO:231).

FIG. 13 illustrates SEQ ID NO:26 depicts the polypeptide sequence of the ectodomain of the prefusion stabilized S protein, including the foldon (italics, underlined), the PP and GSAS (SEQ ID NO:190) substitutions (underlined, bold), but lacking HRV3C protease cleavage, TwinStrepTab, and 8XHisTag (SEQ ID NO:231).

FIG. 14 illustrates details of the study design for the SARSCoV-2 vaccine using the SAM platform in the female BALB/c mouse.

FIG. 15 illustrates Luminex titers of study samples in AU with horizontal bars representing the Geometric Mean Titer (GMT) and vertical bars representing the 95% Confidence Intervals (CI). CNE: CoV-2 Spike_FL-2P SAM (CNE); LNP: CoV-2 Spike_FL-2P SAM (LNP); ecto: CoV-2 Spike _ECTO-2P SAM (LNP).

FIG. 16A illustrates Geometric Mean Ratio of CNE vs. LNP delivery formulations, SAM at 1.5 µg. FIG. 16B illustrates Geometric Mean Ratio of CNE vs. LNP delivery formulations, SAM at 0.15 µg.

FIG. 17A illustrates Geometric Mean Ratio of Spike_FL-2P SAM vs Spike_ECTO-2P SAM formulated with LNP, SAM at 1.5 µg. FIG. 17B illustrates Geometric Mean Ratio of Spike_FL-2P SAM vs Spike_ECTO-2P SAM formulated with LNP, SAM at 0.15 µg.

FIG. 18 illustrates neutralization antibody titers of the study samples in NT50 with horizontal bars representing the Geometric Mean Titer (GMT) and vertical bars representing the 95% Confidence Intervals (CI). CNE: CoV-2 Spike_FL-2P SAM (CNE); LNP:CoV-2 Spike _FL-2P SAM (LNP); ecto: CoV-2 Spike _ECTO-2P SAM (LNP).

FIG. 19A illustrates Geometric Mean Ratio of CNE vs. LNP delivery formulations, SAM at 1.5 µg. FIG. 19B illustrates Geometric Mean Ratio of CNE vs. LNP delivery formulations, SAM at 0.15 µg.

FIG. 20 illustrates Geometric Mean Ratio of Spike_FL-2P SAM vs Spike_ECTO-2P SAM formulated at 0.15 µg with LNP.

FIG. 21A illustrates frequency of IgD-IgM- cells among the CD3-CD19+ B-cells. FIG. 22B illustrates Frequency of CD95+GL7+ cells among the CD3-CD19+IgD-IgM- B cells. Each bar indicates magnitude of the response with the SEM shown in error bars. * indicates a statistical difference with P<0.05.

FIG. 22A illustrates frequency of Spike-specific cells among the CD3-CD19+IgD-IgM- B cells. FIG. 22B illustrates frequency of CD95+CD38+ among the Spike-specific CD3-CD19+IgD-IgM- B-cells. FIG. 22C illustrates frequency of CD95+GL7+ among the Spike-specific CD3-CD19+IgD-IgM- B-cells. Each bar indicates magnitude of the response with the SEM shown in error bars. * indicates a statistical difference with P<0.05.

FIG. 23A illustrates frequency of IgD-IgM- cells among the CD3-CD19+ B-cells. FIG. 23B illustrates frequency of CD95+GL7+ cells among the CD3-CD19+IgD-IgM- B-cells. Each bar indicates magnitude of the response with the SEM shown in error bars. * indicates a statistical difference with P<0.05.

FIG. 24A illustrates frequency of the Spike-specific cells among CD3-CD19+ IgD-IgM- B-cells. FIG. 24B illustrates frequency of CD95+GL7+ cells among Spike-specific CD3-CD19+IgD-IgM- B cells. Each bar indicates magnitude of the response with the SEM shown in error bars. * indicates a statistical difference with P<0.05.

FIG. 25A illustrates frequency of CD73+ cells among the Spike-specific CD3-CD19+IgD-IgM- B-cells. FIG. 25B illustrates frequency of CD80+ cells among the Spike-specific CD3-CD19+IgD-IgM- B-cells. FIG. 25C illustrates frequency of CD273+ cells among the Spike-specific CD3-CD19+IgD-IgM- B-cells. Each bar indicates magnitude of the response with the SEM shown in error bars. Mann-Whitney test was used for a side-by-side statistical comparison between the AS03-adjuvantd and the SAM LNP vaccine groups. ** indicates a statistical difference with P<0.01.

FIG. 26A illustrates the mean and SEM of total spike-specific CD4+ T-cell responses from 5 individual mice per group. FIG. 26B illustrates the mean and SEM of total spike-specific CD8+ T-cell responses from 5 individual mice per group. [*] Denotes a significant difference in total CD8+ T-cell response between the SAM LNP spike FL vs. SAM CNE spike FL vaccine groups at comparable doses. [†] Denotes a significant difference in Total T-cell responses between the spike protein/AS03 group and all SAM vaccine groups, apart from the SAM (CNE) 15 µg and 1.5 µg doses and the SAM (LNP) 1.5 µg dose for CD4 T-cells.

FIG. 27 illustrates mean of various spike-specific polyfunctional CD4+ (top panel) and CD8+ (bottom panel) T-cell populations for select vaccine groups. The dots within each bar represent individual mice responses.

FIG. 28A illustrates SARS-CoV-2 Spike-specific individual cytokine CD107a CD4+ T-cell responses in splenocytes at 2wp2. FIG. 28B illustrates SARS-CoV-2 Spike-specific individual cytokine IFN-γ CD4+ T-cell responses in splenocytes at 2wp2. FIG. 28C illustrates SARS-CoV-2 Spike-specific individual cytokine IL-4/IL-13 CD4+ T-cell responses in splenocytes at 2wp2. FIG. 28D illustrates SARS-CoV-2 Spike-specific individual cytokine IL-2 CD4+ T-cell responses in splenocytes at 2wp2. FIG. 28E illustrates SARS-CoV-2 Spike-specific individual cytokine TNF-α CD4+ T-cell responses in splenocytes at 2wp2. FIG. 28F illustrates SARS-CoV-2 Spike-specific individual cytokine IL-17F CD4+ T-cell responses in splenocytes at 2wp2.

FIG. 29A illustrates SARS-CoV-2 Spike-specific individual cytokine CD107a CD8+ T-cell responses in splenocytes at 2wp2. FIG. 29B illustrates SARS-CoV-2 Spike-specific individual cytokine IFN-γ CD8+ T-cell responses in splenocytes at 2wp2. FIG. 29C illustrates SARS-CoV-2 Spike-specific individual cytokine IL-2 CD8+ T-cell responses in splenocytes at 2wp2. FIG. 29D illustrates SARS-CoV-2 Spike-specific individual cytokine TNF-α CD8+ T-cell responses in splenocytes at 2wp2.

FIG. 30 illustrates the mean and SEM of total SAM nsP-specific CD4+ (top panel) and CD8+ (bottom panel) T-cell responses from 5 individual mice per group as measured using stimulation of splenocytes with a combination of nsP-1, nsP-2, nsP-3, and nsP-4 peptide pools. [*] Denotes a significantly different total T-cell response between comparator groups.

FIG. 31 illustrates frequencies of Tfh cells in spleen represented as mean ± SEM. Significance was determined by one-way ANOVA using GraphPad Prism 8.0. **:P<0.01; ***:P<0.001.

FIG. 32 - SAM-SARS CoV2 construct. The self-amplifying mRNA (SAM) shown herein is based on the RNA backbone of a VEE TC-83 replicon. This SAM comprises from 5′ to 3′ a non-coding sequence; a sequence encoding the viral nonstructural proteins 1-4 (nsP1-4); a subgenomic promoter; an insertion site comprising a construct encoding a CoV2 S antigen; a non-coding sequence and a poly(A) tail. A DNA encoding an empty SAM is shown in SEQ ID NO:170; the corresponding empty SAM is shown in SEQ ID NO: 171. The insertion site is immediately after nucleotide 7561.

FIG. 33A - FIG. 33C - SARS-CoV2 Spike protein optimization (adapted from Wrapp et al., 2020). FIG. 33A) Native full-length SARS-CoV2 Spike (S) protein; FIG. 33B) SAM - Full-length SARS-CoV2 Spike (S) protein (2XP,GSAS) (pJW19) FIG. 33B discloses “GSAS” as SEQ ID NO: 190. FIG. 33C) SAM - Ecto domain SARS-CoV2 Spike (S) protein (2XP,GSAS,T4 Trimer motif) (pJW18). FIG. 33C discloses “GSAS” as SEQ ID NO: 190. NTD: N-terminal domain; RBD: Receptor binding domain; FP: Fusion peptide; HR1: Heptad repeat 1; HR2: Heptad repeat 2; CH: Central helix; TM: Transmembrane; CT: C-terminal; CD: Connector domain; S2′: S2′ protease cleavage site; S1/S2: Cleavage site between S1 and S2 (furin-like).

FIG. 34 - Mutation of the S protein signal sequence in full length (FL) S protein (pJW19). FIG. 34 discloses SEQ ID NOS 190 and 232-254, respectively, in order of appearance.

FIG. 35 - Mutation of the S protein signal sequence in Ecto domain S protein (pJW18). FIG. 35 discloses SEQ ID NOS 190 and 232-254, respectively, in order of appearance.

FIG. 36 - Mutation of the ER retention signal in full length (FL) S protein (pJW19). FIG. 36 discloses SEQ ID NOS 190 and 255-257, respectively in order of appearance.

FIG. 37: SEQ ID NO:49 depicts the polypeptide sequence of the full-length S protein, including the PP and GSAS (SEQ ID NO: 190) substitutions (underlined, bold) of Wrapp et al. (2020), but lacking the foldon, HRV3C protease cleavage, TwinStrepTab, and 8XHisTag (SEQ ID NO: 231).

FIG. 38: SEQ ID NO:26 depicts the polypeptide sequence of the ectodomain of the prefusion stabilized S protein, including the foldon (italics, underlined), the PP and GSAS (SEQ ID NO:190) substitutions (underlined, bold) of Wrapp et al. (2020), but lacking HRV3C protease cleavage, TwinStrepTab, and 8XHisTag (SEQ ID NO: 231).

FIG. 39: DNA sequence of the plasmid that expresses the RNA sequence for the SAM-SARS-CoV2 Spike constructs. Upper case: SAM backbone; Lower case: non-SAM sequence; underlined: 5′ UTR of SAM; bold underlined: 3′ UTR of SAM; grey shade: antigen insertion site. FIG. 39 discloses SEQ ID NO: 170.

FIG. 40 illustrates % of antigen positive BHK cells (100 ng RNA electroporation). SARS-CoV-2 spike full length SAM mutants (heterologous signal sequences) compared to WT, i.e., pJW19 encoding the native spike protein signal sequence.

FIG. 41 illustrates % of antigen positive BHK cells (100 ng RNA electroporation). SARS-CoV-2 spike full length SAM mutants (heterologous signal sequences).

FIG. 42 illustrates MFI of spike positive BHK cells (100 ng RNA electroporation). SARS-CoV-2 spike full length SAM mutants (heterologous signal sequences) compared to WT, i.e., pJW19 encoding the native spike protein signal sequence.

FIG. 43 illustrates % of antigen positive BHK cells (300 ng RNA electroporation). SARS-CoV-2 spike full length SAM mutants (heterologous signal sequences) compared to WT, i.e., pJW19 encoding the native spike protein signal sequence.

FIG. 44 illustrates % of antigen positive BHK cells (2 µg RNA electroporation). SARS-CoV-2 spike full length SAM mutants (heterologous signal sequences) compared to WT, i.e., pJW19 encoding the native spike protein signal sequence.

FIG. 45 illustrates % of antigen positive BHK cells (100 ng RNA electroporation). SARS-CoV-2 spike Ecto SAM mutants (heterologous signal sequences) compared to WT, i.e., pJW18 encoding the native spike protein signal sequence.

FIG. 46 illustrates concentration of spike protein in concentrated supernatant. SARS-CoV-2 spike Ecto SAM mutants (heterologous signal sequences) compared to WT, i.e., pJW18 encoding the native spike protein signal sequence.

FIG. 47 illustrates concentration of spike protein in cell lysate. SARS-CoV-2 spike Ecto SAM mutants (heterologous signal sequences) compared to WT, i.e., pJW18 encoding the native spike protein signal sequence.

FIG. 48 illustrates concentration of spike protein in cell lysate and supernatant. SARS-CoV-2 spike Ecto SAM mutants (heterologous signal sequences) compared to WT, i.e., pJW18 encoding the native spike protein signal sequence.

FIG. 49 illustrates supernatant-to-cell ratio of spike protein. SARS-CoV-2 spike Ecto SAM mutants (heterologous signal sequences) compared to WT, i.e., pJW18 encoding the native spike protein signal sequence.

FIG. 50 illustrates supernatant-to-cell ratio of spike protein normalized to actin and wild type. SARS-CoV-2 spike Ecto SAM mutants (heterologous signal sequences) compared to WT, i.e., pJW18 encoding the native spike protein signal sequence.

### DETAILED DESCRIPTION OF THE INVENTION

### Antigens; Variants; Fragments; and Constructs

The present inventors provide constructs, RNA molecules, and self-replicating RNA molecules useful as components of immunogenic compositions for the induction of an immune response in a subject, nucleic acids useful for their expression, methods for their use in treatment, and processes for their manufacture. In some embodiments, the present inventors provide constructs and self-replicating RNA molecules useful as components of immunogenic compositions for the induction of an immune response in a subject against Coronaviral infection, nucleic acids useful for their expression, methods for their use in treatment, and processes for their manufacture. By construct is intended a nucleic acid that encodes polypeptide sequences described herein, and may comprise DNA, RNA, or non-naturally occurring nucleic acid monomers. The nucleic acid components of constructs are described more fully in the Nucleic Acids section herein.

In some embodiments, the constructs, RNA molecules, and and self-replicating RNA molecules disclosed herein encode wild-type polypeptide sequences of a Coronavirus, or a variant, or a fragment thereof. The constructs and self-replicating RNA molecules may further encode a polypeptide sequence heterologous to the polypeptide sequences of a Coronavirus. In some embodiments, the constructs and self-replicating RNA molecules encode wild-type polypeptide sequences of a SARS CoV-2, or a variant, or a fragment thereof. Unless indicated otherwise, descriptions of the wild-type Coronavirus antigens are made by reference to those encoded by the genome of the 2019-nCoV virus (GenBank: MN908947).

A “variant” of a polypeptide sequence includes amino acid sequences having one or more amino acid substitutions and/or deletions when compared to the reference sequence. In some embodiments, a variant includes the relevant polypeptide from a NL63-CoV, 229E-CoV, OC43-CoV, SARS-CoV, MERS-CoV, HKUI-CoV, WIVI-CoV, mouse hepatitis virus (MHV), or HKU9-CoV. In some embodiments, the variant may comprise an amino acid sequence which is at least 70%, at least 75%, at least 80%, at least 85%, at least 90%, at least 95%, at least 96%, at least 97%, at least 98%, or at least 99%) identical to a full-length wild-type polypeptide. Alternatively, or in addition, a fragment of a polypeptide may comprise an immunogenic fragment (i.e. an epitope-containing fragment) of the full-length polypeptide which may comprise a contiguous amino acid sequence of at least 8, at least 9, at least 10, at least 11, at least 12, at least 13, at least 14, at least 15, at least 16, at least 17, at least 18, at least, or more amino acids which is identical to a contiguous amino acid sequence of the full-length polypeptide. As used herein, the term “antigen” refers to a molecule containing one or more epitopes (e.g., linear, conformational or both) that will stimulate a host’s immune system to make a humoral and/or cellular antigen-specific immunological response (i.e. an immune response which specifically recognizes a naturally occurring polypeptide). An “epitope” is that portion of an antigen that determines its immunological specificity.

T- and B-cell epitopes can be identified empirically (e.g. using PEPSCAN or similar methods). See the following reference: Geysen et al. (1984) PNAS USA 81 :3998-4002; Carter (1994) Methods Mol Biol 36:207-23. They can be predicted (e.g. using the Jameson- Wolf antigenic index (see Jameson et al. (1988) CABIOS 4(1): 181-186), matrix-based approaches (see Raddrizzani & Hammer (2000) Brief Bioinform 1(2): 179-89), TEPITOPE (see De Lalla et al. (1999) J. Immunol. 163: 1725-29), neural networks (see Brusic et al. (1998) Bioinformatics 14(2): 121-30), OptiMer & EpiMer (see Meister et al. (1995) Vaccine 13(6):581-91; see Roberts et al. (1996) AIDS Res Hum Retroviruses 12(7):593-610), ADEPT (see Maksyutov & Zagrebelnaya (1993) Comput Appl Biosci 9(3):291-7), Tsites (see Feller & de la Cruz (1991) Nature 349(6311):720-1), hydrophilicity (see Hopp (1993) Peptide Research 6:183-190), antigenic index (see Welling et al. (1985) FEBS Lett. 188:215-218) or the methods disclosed in reference Davenport et al. (1995) Immunogenetics 42:392-297, etc.).

In some embodiments, constructs and self-replicating RNA molecules are provided herein that encode a Coronavirus S antigen. By “Coronavirus S antigen” is intended the amino acid sequence, or a nucleotide sequence encoding the amino acid sequence, of a wild-type Coronavirus S protein, variant, or fragment thereof. Unless indicated otherwise, descriptions of the wild-type S antigen are made by reference to residues 1-1273 encoded by the genome of 2019-nCoV S (GenBank: MN908947), as depicted in the SEQ ID NO:11 (polypeptide).

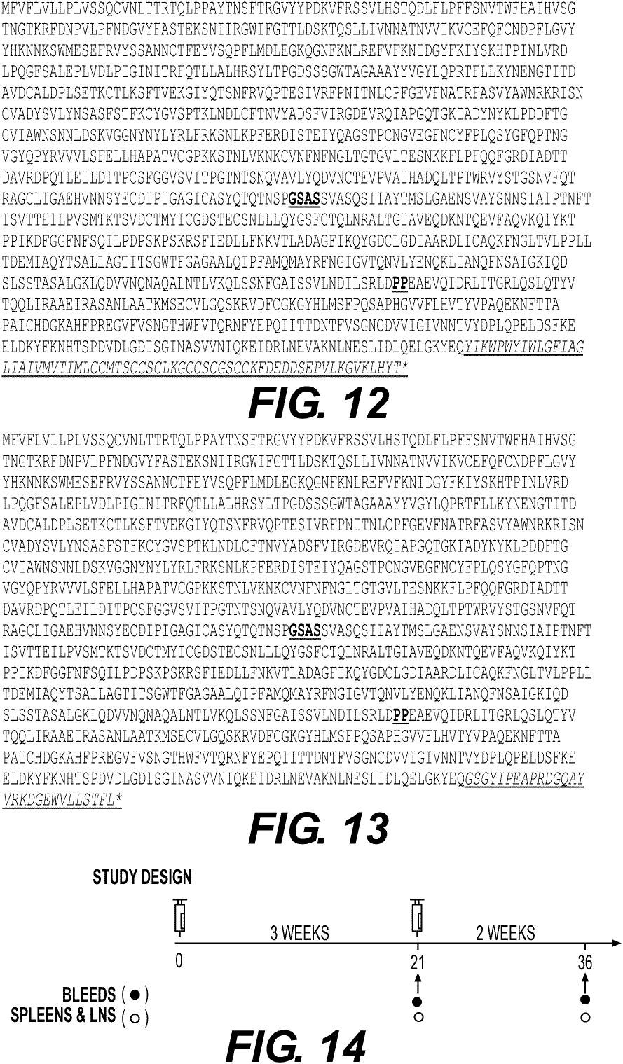

In some embodiments, the constructs and self-replicating RNA molecules encode a prefusion stabilized Coronavirus S protein variant as described in WO2018081318. In some embodiments, the constructs and self-replicating RNA molecules encode a recombinant coronavirus S antigen comprising one or more proline substitution(s) that stabilize the S protein trimer in the prefusion conformation. Such as one or more (such as two) proline substitutions at or near the boundary between a Heptad Repeat 1 (HR1) and a central helix of the protomers of the coronavirus S ectodomain trimer was found to be surprisingly effective for stabilization of coronavirus S protein trimers in the prefusion conformation. In some embodiments, the constructs and self-replicating RNA molecules encode a recombinant alphacoronavirus or betacoronavirus S antigen comprising one or two proline substitutions at or near a junction between a heptad repeat 1 (HR1) and a central helix that stabilizes the S trimer in a prefusion conformation. The one or two proline substitutions can comprise two consecutive proline substitutions (a “double proline substitution”). FIG. 12, FIG. 13, and FIG. 38 disclose the amino acid sequence of two such prefusion stabilized Coronavirus S protein variants. The sequence identifier numbers for each are set forth in the Sequences section and Sequence Listing herein. See SEQ ID NOS:49 and SEQ ID NO:26. The S antigen can also comprise a mutation in the furin cleavage site to help stabilize the prefusion form of the protein. Without wishing to be bound by theory, It is also considered that mutating the furin cleavage site will avoid the protein being processed into two subunits inside the cell and enable the entire protein to be expressed on the cell surface or be secreted. In some embodiments, the ecto domain protein has the entire C-terminus (including the transmembrane domain) deleted and replaced by a trimerization domain such as the T4 fibritin trimerization (foldon) motif, in order to promote the formation of trimeric complexes and stabilize the prefusion form of the protein.

Thus, in embodiments where a Coronavirus S protein (antigen) is a variant of a prefusion stabilized S polypeptide, the variant may be a stabilized NL63-CoV, 229E-CoV, OC43-CoV, SARS-CoV, MERS-CoV, HKUI-CoV, WIVI-CoV, mouse hepatitis virus (MHV), or HKU9-CoV S protein variant as described in WO2018081318, or a stabilized SARS CoV-2 S protein variant as described in Wrapp (2020) “Cryo-EM structure of the 2019-nCoV spike in the prefusion conformation.” Science, 367:1260-1263.

In some embodiments, a construct encodes a stabilized SARS CoV-2 S protein variant. In some embodiments, a construct may encode a polypeptide having a sequence as set forth in SEQ ID NO:49 or SEQ ID NO:26. In some embodiments, a construct may encode a polypeptide having is at least 70%, at least 75%, at least 80%, at least 85%, at least 90%, at least 95%, at least 96%, at least 97%, at least 98%, or at least 99% identical to a sequence set forth in SEQ ID NOS:49 or SEQ ID NO:26. In some embodiments, a construct may encode a polypeptide which comprises a fragment of a full-length sequence set forth in SEQ ID NOS:49 or SEQ ID NO:26, wherein the fragment comprises a contiguous stretch of the amino acid sequence of the full-length sequence up to 1, 2, 3, 4, 5, 6, 7, 8, 9, or 10 amino acids shorter than full-length sequence. In some embodiments, a fragment or variant described herein can be immunogenic.

In some embodiments, the construct comprises a DNA nucleic acid sequence set forth in SEQ ID NO:97 or SEQ ID NO:74. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence set forth in SEQ ID NO:97 or SEQ ID NO:74. In such embodiments, the construct comprises a nucleic acid sequence which comprises a fragment of a full-length sequence set forth in SEQ ID NO:97 or SEQ ID NO:74 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, the construct comprises a RNA nucleic acid sequence comprising a sequence set forth in SEQ ID NO:145 or SEQ ID NO:122. In such embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence set forth in SEQ ID NO:145 and SEQ ID NO:122. In such embodiments, the construct comprises a nucleic acid sequence which comprises a fragment of a full-length sequence selected from the group consisting of SEQ ID NO:145 and SEQ ID NO:122 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

### Signal Sequence

The present invention aims in particular at enhancing the expression and secretion or cell-surface expression of an antigen expressed from an intracellular construct. The inventors have in particular designed constructs in which the native antigen signal sequence is replaced by a heterologous signal sequence. The inventors have also designed constructs in which the endoplasmic reticulum (ER) retention signal is mutated or deleted.

In one aspect, the invention provides a construct comprising a nucleic acid sequence encoding an antigen, wherein the antigen comprises a heterologous signal sequence. In one aspect, the invention provides a construct comprising a nucleic acid sequence encoding an antigen, wherein the antigen comprises a heterologous signal sequence, a mutation with respect to the wild-type sequence which affects retention of the antigen in the endoplasmic reticulum, or a combination thereof.

In one embodiment, the antigen is a coronavirus antigen. The coronavirus antigen may be from SARS CoV-2, NL63-CoV, 229E-CoV, OC43-CoV, SARS-CoV, MERS-CoV, HKUI-CoV, WIVI-CoV, mouse hepatitis virus (MHV), HKU9-CoV S, or a variant thereof. Suitably, the coronavirus antigen is a coronavirus S protein. The coronavirus S protein may be a S protein from SARS CoV-2, NL63-CoV, 229E-CoV, OC43-CoV, SARS-CoV, MERS-CoV, HKUI-CoV, WIVI-CoV, mouse hepatitis virus (MHV), HKU9-CoV S, or a variant thereof. In a preferred embodiment, the coronavirus S protein is a SARS CoV-2 S protein. In some embodiments, a variant disclosed herein is immunogenic.

In some embodiments, the antigen comprises a heterologous signal sequence. As used herein, a “signal sequence” refers to a short (usually less than 60 amino acids, for example, 3 to 60 amino acids) amino acid sequence present on precursor proteins (typically at the N terminus), and which is typically absent from the mature protein. The signal sequence is typically rich in hydrophobic amino acids. The signal peptide directs the transport and/or secretion of the translated protein through the membrane. Signal sequences may also be called targeting signals, transit peptides, localization signals, or signal peptides. A “heterologous signal sequence” is a signal sequence which originates from a different species than the antigen. In some embodiments, a heterologous signal sequence comprises a positive charge at its N terminus.

Suitably, the heterologous signal sequence has a sequence selected from:
a) a Gaussian Luciferase signal sequence as shown in SEQ ID NO:2,
b) a human CD5 signal sequence as shown in SEQ ID NO:3,
c) a human CD33 signal sequence as shown in SEQ ID NO:4,
d) a human IL2 signal sequence as shown in SEQ ID NO:5,
e) a human IgE signal sequence as shown in SEQ ID NO:6,
f) a human Light Chain Kappa signal sequence as shown in SEQ ID NO:7,
g) a JEV short signal sequence as shown in SEQ ID NO:8,
h) a JEV long signal sequence as shown in SEQ ID NO:9,
i) a Mouse Light Chain Kappa signal sequence as shown in SEQ ID NO:10,
j) a SSP signal sequence as shown in SEQ ID NO:11,
k) a Gaussian Luciferase (AKP) signal sequence as shown in SEQ ID NO: 12, and
l) a variant of any one of sequences (a)-(k) having 1, 2, 3, 4 or 5 amino acid residue deletions, insertions or substitutions.

In some embodiments, the heterologous signal sequence has a sequence selected from:
a) a Gaussian Luciferase signal sequence as shown in SEQ ID NO:2,
b) a human Light Chain Kappa signal sequence as shown in SEQ ID NO:7,
c) a Gaussian Luciferase (AKP) signal sequence as shown in SEQ ID NO: 12, and
d) a variant of any one of sequences (a)-(k) having 1, 2, 3, 4 or 5 amino acid residue deletions, insertions or substitutions.

In some embodiments, the heterologous signal sequence comprises a sequence which is at least 70%, at least 75%, at least 80%, at least 85%, at least 90%, at least 95%, at least 96%, at least 97%, at least 98%, or at least 99% identical to a sequence selected from: SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO: 12. In some embodiments, the heterologous signal sequence comprises a sequence which is at least 70%, at least 75%, at least 80%, at least 85%, at least 90%, at least 95%, at least 96%, at least 97%, at least 98%, or at least 99% identical to a sequence selected from: SEQ ID NO:2, SEQ ID NO:7, or SEQ ID NO:12. In embodiments where the antigen itself has a naturally occurring signal sequence, the heterologous signal sequence can replace the naturally occurring signal sequence. In one embodiment where the antigen has a naturally occurring signal sequence, the heterologous signal sequence replaces residues 1-16 of the antigen. In one embodiment where the antigen has a naturally occurring signal sequence, the heterologous signal sequence replaces residues 1-18 of the antigen.

In one embodiment where the antigen is a SARS CoV-2 S protein, the heterologous signal sequence replaces residues 1-16 of the SARS CoV-2 S protein as shown in SEQ ID NO:1, or corresponding residues in another SARS CoV-2 S protein sequence. Suitably, residues 1-16 of the SARS CoV-2 S protein as shown in SEQ ID NO:1, or corresponding residues in another SARS CoV-2 S protein sequence are replaced by a sequence selected from:
a) a Gaussian Luciferase signal sequence as shown in SEQ ID NO:2,
b) a human CD5 signal sequence as shown in SEQ ID NO:3,
c) a human CD33 signal sequence as shown in SEQ ID NO:4,
d) a human IL2 signal sequence as shown in SEQ ID NO:5,
e) a human IgE signal sequence as shown in SEQ ID NO:6,
f) a human Light Chain Kappa signal sequence as shown in SEQ ID NO:7,
g) a JEV short signal sequence as shown in SEQ ID NO:8,
h) a JEV long signal sequence as shown in SEQ ID NO:9,
i) a Mouse Light Chain Kappa signal sequence as shown in SEQ ID NO:10,
j) a SSP signal sequence as shown in SEQ ID NO:11,
k) a Gaussian Luciferase (AKP) signal sequence as shown in SEQ ID NO: 12, and
l) a variant of any one of sequences (a)-(k) having 1, 2, 3, 4 or 5 amino acid residue deletions, insertions or substitutions.

In one embodiment where the antigen is a SARS CoV-2 S protein, the heterologous signal sequence replaces residues 1-18 of the SARS CoV-2 S protein as shown in SEQ ID NO:1, or corresponding residues in another SARS CoV-2 S protein sequence. Suitably, residues 1-18 of the SARS CoV-2 S protein as shown in SEQ ID NO:1, or corresponding residues in another SARS CoV-2 S protein sequence are replaced by a sequence selected from:
a) a Gaussian Luciferase signal sequence as shown in SEQ ID NO:2,
b) a human CD5 signal sequence as shown in SEQ ID NO:3,
c) a human CD33 signal sequence as shown in SEQ ID NO:4,
d) a human IL2 signal sequence as shown in SEQ ID NO:5,
e) a human IgE signal sequence as shown in SEQ ID NO:6,
f) a human Light Chain Kappa signal sequence as shown in SEQ ID NO:7,
g) a JEV short signal sequence as shown in SEQ ID NO:8,
h) a JEV long signal sequence as shown in SEQ ID NO:9,
i) a Mouse Light Chain Kappa signal sequence as shown in SEQ ID NO:10,
j) a SSP signal sequence as shown in SEQ ID NO:11,
k) a Gaussian Luciferase (AKP) signal sequence as shown in SEQ ID NO: 12, and
l) a variant of any one of sequences (a)-(k) having 1, 2, 3, 4 or 5 amino acid residue deletions, insertions or substitutions.

In one embodiment, the antigen comprises a mutation with respect to the wild-type sequence which affects retention of the antigen in the endoplasmic reticulum (ER). In one embodiment, one or more point mutations are made to the ER retention signal. In one embodiment, the ER retention signal is deleted. In one embodiment, the entire C-terminal domain comprising the ER retention signal is deleted.

In one embodiment where the antigen is a SARS CoV-2 S protein, the mutation with respect to the wild-type sequence which affects retention of the antigen in the endoplasmic reticulum is selected from:
a) the substitution of residues K1269 and H1271 as shown in SEQ ID NO:1 to alanine residues, or corresponding substitutions in another SARS Cov-2 S protein sequence, and
b) the deletion of residues 1261-1273 of SEQ ID NO:1, or of corresponding residues in another SARS Cov-2 S protein sequence.

In one embodiment where the antigen is a coronavirus S protein, the coronavirus S protein comprises one or mutations with respect to the wild-type sequence which stabilize the prefusion form of the coronavirus S protein. In some embodiments, the construct encodes a prefusion stabilized coronavirus S protein variant as described in WO2018081318. In some embodiments, the construct encodes a recombinant coronavirus S antigen comprising one or more proline substitution(s) that stabilize the S protein trimer in the prefusion conformation. In some embodiments, the construct encodes a recombinant coronavirus S antigen comprising one or more (such as two) proline substitutions at or near the boundary between a Heptad Repeat 1 (HR1) and a central helix that stabilizes the S trimer in a prefusion conformation. In some embodiments, the construct encodes a recombinant alphacoronavirus or betacoronavirus S antigen comprising one or two proline substitutions at or near a junction between a heptad repeat 1 (HR1) and a central helix that stabilizes the S trimer in a prefusion conformation. The one or two proline substitutions can comprise two consecutive proline substitutions (a “double proline substitution”). The S antigen can also comprise a mutation in the furin cleavage site to help stabilize the prefusion form of the protein. In some embodiments, the ecto domain protein has the entire C-terminus (including the transmembrane domain) deleted and replaced by a trimerization domain such as the T4 fibritin trimerization (foldon) motif, in order to promote the formation of trimeric complexes and stabilize the prefusion form of the protein.

In one embodiment where the coronavirus S protein is a SARS CoV-2 S protein, the one or more mutations comprise the substitutions of residues ⁹⁸⁸KV⁹⁸⁷ as shown in SEQ ID NO:1 to ⁹⁸⁶PP⁹⁸⁷, and/or the substitution of residues ⁶⁸²RRAR⁶⁸⁵ (SEQ ID NO:188) as shown in SEQ ID NO:1 to ⁶⁸²GSAS⁶⁸⁵ (SEQ ID NO:190) or corresponding mutations in another SARS Cov-2 S protein sequence.

Suitably, the native transmembrane and cytosolic domains of the coronavirus S protein are replaced by a heterologous trimerization domain. In one embodiment, the native transmembrane and cytosolic domains of the coronavirus S protein are replaced by a trimerization domain, for example a C-terminal T4 fibritin trimerization (foldon) motif.

In one embodiment where the antigen is a SARS CoV-2 S protein, residues 1208-1273 of the sequence shown in SEQ ID NO: 1, or corresponding residues in another SARS Cov-2 S protein sequence, are replaced by a C-terminal T4 fibritin trimerization (foldon) motif having the sequence shown in SEQ ID NO:24.

In one embodiment where the antigen is a SARS CoV-2 S protein, the S protein has an amino acid sequence selected from SEQ ID NOs:27-73, or a variant which is at least 70%, at least 75%, at least 80%, at least 85%, at least 90%, at least 95%, at least 96%, at least 97%, at least 98%, or at least 99% identical thereto.

In one embodiment where the antigen is a SARS CoV-2 S protein, the S protein is encoded by a DNA sequence having a sequence selected from SEQ ID NOs:75-121, or a variant which is at least 70%, at least 75%, at least 80%, at least 85%, at least 90%, at least 95%, at least 96%, at least 97%, at least 98%, or at least 99% identical thereto.

In one embodiment where the antigen is a SARS CoV-2 S protein, the S protein is encoded by an RNA sequence having a sequence selected from SEQ ID NOs:123-169, or a variant which is at least 70%, at least 75%, at least 80%, at least 85%, at least 90%, at least 95%, at least 96%, at least 97%, at least 98%, or at least 99% identical thereto.

In one aspect, the invention provides a nucleic acid sequence comprising a construct as described above, and further comprising additional sequence elements. For instance, the nucleic acid may comprise sequence elements useful for the functioning of a mRNA, a self-replicating RNA, a plasmid, or the like.

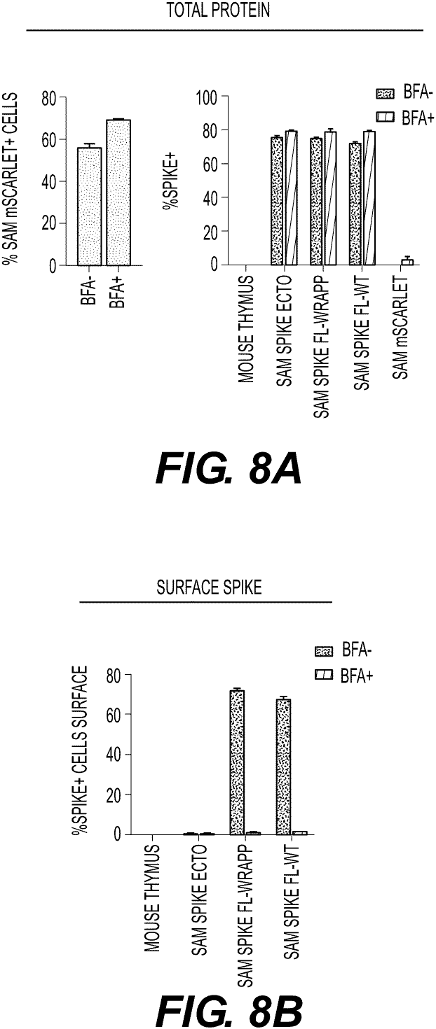

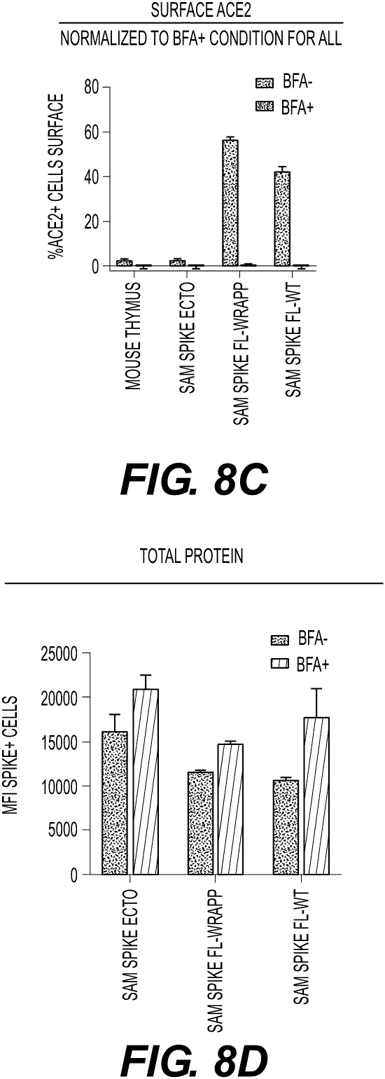

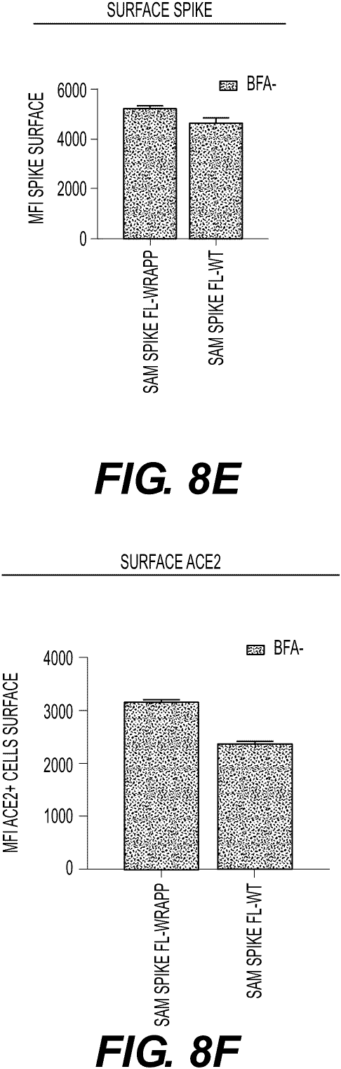

In one embodiment of the invention, the nucleic acid comprises a construct according to the invention and additional sequence elements useful for the functioning of a mRNA, and the nucleic acid is an RNA molecule. In one embodiment, the RNA is a self-replicating RNA. Suitably, the self-replicating RNA comprises or consists essentially of a VEE TC-83 replicon encoding from 5′ to 3′ viral nonstructural proteins 1-4 (nsP1-4), followed by a subgenomic promoter, and a construct (or insert) encoding the antigen. In one embodiment, the VEE TC-83 replicon has the DNA sequence shown in FIG. 8 and SEQ ID NO:170, and the construct encoding the antigen is inserted immediately after residue 7561. In one embodiment, the VEE TC-83 replicon has the RNA sequence shown in SEQ ID NO:171, and the construct encoding the antigen is inserted immediately after residue 7561. In one embodiment, the self-replicating RNA comprises from 5′ to 3′ a sequence having SEQ ID NO:172, a construct encoding (i) a signal sequence selected from the group consisting of SEQ ID NOS:258-268 and (ii) and antigen, and a sequence having SEQ ID NO: 173. In one embodiment, the self-replicating RNA comprises from 5′ to 3′ a sequence having SEQ ID NO:172, a construct encoding (i) a signal sequence selected from the group consisting of SEQ ID NO:258, SEQ ID NO:263, and SEQ ID NO:268 and (ii) and antigen, and a sequence having SEQ ID NO:173. In one embodiment, the self-replicating RNA comprises from 5′ to 3′ a sequence having SEQ ID NO:172, a construct having a sequence selected from the group consisting of SEQ ID NOS:122-169, and a sequence having SEQ ID NO: 173. In one embodiment, the antigen is a coronavirus antigen, preferably a SARS-CoV2 S protein.

The invention also provides a DNA molecule encoding the RNA molecule of the invention. In some embodiments, the DNA molecule comprises SEQ ID NO:174 and/or SEQ ID NO:175. In some embodiments, a DNA encoding a SAM may comprise three regions from 5′ to 3′, the first region comprising the sequence up to the insertion point (for instance nucleotides 1-7561 of SEQ ID NO:170, herein SEQ ID NO:174), the second region comprising a construct (for example comprising an immunogenic antigen disclosed herein), and the third region comprising the sequence after the insertion point (for instance nucleotides 7562-10000 of SEQ ID NO:170, herein SEQ ID NO:175).

In some embodiments, the invention provides a construct encoding a polypeptide having a sequence selected from the group consisting of SEQ ID NOS:26-73. In some embodiments, the construct encodes a polypeptide which is at least 70%, at least 75%, at least 80%, at least 85%, at least 90%, at least 95%, at least 96%, at least 97%, at least 98%, or at least 99% identical to a sequence selected from the group consisting of SEQ ID NOS:26-73. In some embodiments, the construct encodes a polypeptide which comprises a fragment of a full-length sequence selected from the group consisting of SEQ ID NOS:26-73, wherein the fragment comprises a contiguous stretch of the amino acid sequence of the full-length sequence up to 1, 2, 3, 4, 5, 6, 7, 8, 9, or 10 amino acids shorter than full-length sequence.

In some embodiments, the construct comprises a DNA nucleic acid sequence selected from the group consisting of SEQ ID NOS:74-121. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NOS:74-121. In some embodiments, the construct comprises a nucleic acid sequence which comprises a fragment of a full-length sequence selected from the group consisting of SEQ ID NOS:74-121 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, the construct comprises a RNA nucleic acid sequence selected from the group consisting of SEQ ID NOS:122-169. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NOS:122-169. In some embodiments, the construct comprises a nucleic acid sequence which comprises a fragment of a full-length sequence selected from the group consisting of SEQ ID NOS:122-169 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

### Nucleic Acids

The present inventors disclose herein nucleic acid comprising a sequence which encodes a Coronavirus S antigen. Nucleic acid as disclosed herein can take various forms (e.g. single-stranded, double-stranded, vectors etc.). Nucleic acids may be circular or branched, but will generally be linear.

The nucleic acids used herein are preferably provided in purified or substantially purified form i.e. substantially free from other nucleic acids (e.g. free from naturally-occurring nucleic acids), particularly from other Coronavirus or host cell nucleic acids, generally being at least about 50% pure (by weight), and usually at least about 90% pure.

Nucleic acids may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap.

The nucleic acids herein comprise a sequence which encodes at least one Coronavirus S antigen. Typically, the nucleic acids of the invention will be in recombinant form, i. e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the sequence encoding at least one Coronavirus S antigen. The nucleic acid may be part of a vector i.e. part of a nucleic acid designed for transduction/transfection of one or more cell types. Vectors may be, for example, “expression vectors” which are designed for expression of a nucleotide sequence in a host cell, or “viral vectors” which are designed to result in the production of a recombinant virus or virus-like particle.

Alternatively, or in addition, the sequence or chemical structure of the nucleic acid may be modified compared to a naturally-occurring sequence which encodes a Coronavirus S antigen. The sequence of the nucleic acid molecule may be modified, e.g. to increase the efficacy of expression or replication of the nucleic acid, or to provide additional stability or resistance to degradation.

The nucleic acid encoding the polypeptides described above may be codon optimized. In some embodiments, the nucleic acid encoding the polypeptides described above may be codon optimized for expression in human cells. By “codon optimized” is intended modification with respect to codon usage may increase translation efficacy and half- life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl- transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2 ‘-O-Methyltransferase which results in the generation of a cap 1 structure (m7Gppp [m2 ‘-O] N), which may further increases translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. The preparation of nucleotides and modified nucleotides and nucleosides are well-known in the art, see the following references: U.S. Pat. No. 4373071, 4458066, 4500707, 4668777, 4973679, 5047524, 5132418, 5153319, 5262530, 5700642. Many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-0-methyluridine), mlA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); mil (1-methylinosine); m′lm (I,2′-0-dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-0-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); £5Cm (5-formyl-2′-0-methylcytidine); mlGm (I, 2′-0-dimethylguanosine); m′Am (1, 2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(Ci-C6 )-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-0-methyl-U. Many of these modified nucleobases and their corresponding ribonucleosides are available from commercial suppliers.

### Nucleic Acid-Based Vaccines

The present inventors disclose compositions comprising a nucleic acid sequence which encodes a polypeptide antigen, variant or fragment thereof, i.e., a construct as described elsewhere herein. The present inventors disclose compositions comprising a nucleic acid sequence which encodes a polypeptide comprising a Coronavirus antigen, variant or fragment thereof, i.e., a construct as described elsewhere herein. Such compositions may be a nucleic acid-based vaccine. A further composition comprising a nucleic acid sequence which encodes one or more additional Coronavirus antigens may also be provided as a nucleic acid-based vaccine. In some embodiments, a composition comprises a nucleic acid sequence encoding a Coronavirus S antigen from a first Coronavirus strain and an additional nucleic acid sequence encoding an additional Coronavirus S antigen from one or more other strains of Coronavirus. In some embodiments, a composition comprises a nucleic acid sequence encoding a Coronavirus S antigen and an additional Coronavirus antigen. Alternatively, an additional non-Coronavirus antigen may be encoded.

The nucleic acid may, for example, be RNA (i. e. an RNA-based vaccine) or DNA (i. e. a DNA-based vaccine, such as a plasmid DNA vaccine). In certain embodiments, the nucleic acid-based vaccine is an RNA-based vaccine. In certain embodiments, the RNA-based vaccine comprises a self-replicating RNA molecule. The self-replicating RNA molecule may be an alphavirus-derived RNA replicon.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded antigen (i.e. a Coronavirus prM-F antigen), or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

One suitable system for achieving self-replication in this manner is to use an alphavirus-based replicon. These replicons are +-stranded RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These - -strand transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) a RNA-dependent RNA polymerase which can transcribe RNA from the self-replicating RNA molecule and (ii) a Coronavirus S antigen. The polymerase can be an alphavirus replicase e.g. comprising one or more of alphavirus proteins nsPI, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild- type viruses are absent from self-replicating RNAs of the present disclosure and their place is taken by gene(s) encoding the immunogen of interest, such that the subgenomic transcript encodes the immunogen rather than the structural alphavirus virion proteins.

Thus a self-replicating RNA molecule useful with the invention may have two open reading frames. The first (5′) open reading frame encodes a replicase; the second (3′) open reading frame encodes an antigen. In some embodiments the RNA may have additional (e.g. downstream) open reading frames e.g. to encode further antigens or to encode accessory polypeptides.

A DNA encoding an empty SAM is shown in FIG. 8 and SEQ ID NO:170; the corresponding empty SAM is shown in SEQ ID NO:171. A construct would be inserted immediately after nucleotide 7561. Thus, a SAM may comprise three regions from 5′ to 3′, the first region comprising the sequence up to the insertion point (for instance nucleotides 1-7561 of SEQ ID NO:171, herein SEQ ID NO:172), the second region comprising a construct, and the third region comprising the sequence after the insertion point (for instance nucleotides 7562-7747 of SEQ ID NO:171, herein SEQ ID NO:173). Thus, a DNA encoding a SAM may comprise three regions from 5′ to 3′, the first region comprising the sequence up to the insertion point (for instance nucleotides 1-7561 of SEQ ID NO:170, herein SEQ ID NO:174), the second region comprising a construct, and the third region comprising the sequence after the insertion point (for instance nucleotides 7562-10000 of SEQ ID NO:170, herein SEQ ID NO: 175).

In certain embodiments, the self-replicating RNA molecule disclosed herein has a 5′ cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly- A polymerase recognition sequence (e.g. AAUAAA) near its 3′ end.

Self-replicating RNA molecules can have various lengths, but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon’s poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. A RNA used with the invention ideally includes only phosphodiester linkages between nucleosides, but in some embodiments it can contain phosphoramidate, phosphorothioate, and/or methylphosphonate linkages.

The self-replicating RNA molecule may encode a single heterologous polypeptide antigen (i. e. a Coronavirus S antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more Coronavirus antigens (e.g. one, two or more Coronavirus S antigens) together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes a Coronavirus S antigen. In addition, T helper cell differentiation can be analyzed by measuring proliferation or production of TH1 (IL-2 and IFN-γ) and /or TH2 (IL-4 and IL-5) cytokines by ELISA or directly in CD4+ T cells by cytoplasmic cytokine staining and flow cytometry.

Self-replicating RNA molecules that encode a Coronavirus S antigen can also be tested for ability to induce humoral immune responses, as evidenced, for example, by induction of B cell production of antibodies specific for a Coronavirus S antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded Coronavirus S antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In some embodiments, the self-replicating RNA molecules may comprise a sequence selected from SEQ ID NO:176 or SEQ ID NO:178. In some embodiments, the self-replicating RNA molecules comprise a sequence which is at least 70%, at least 75%, at least 80%, at least 85%, at least 90%, at least 95%, at least 96%, at least 97%, at least 98%, or at least 99% identical to a sequence selected SEQ ID NO: 176 or SEQ ID NO: 178. In some embodiments, the self- replicating RNA molecule comprises a fragment of a full-length sequence selected from SEQ ID NO: 176 or SEQ ID NO: 178 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, the self-replicating RNA molecules comprise from 5′ to 3′ a sequence having SEQ ID NO:172, a construct having a sequence selected from the group consisting of SEQ ID NOS:122-169, and a sequence having SEQ ID NO:173. In some embodiments, the self-replicating RNA molecules comprise from 5′ to 3′ a sequence which is at least 70%, at least 75%, at least 80%, at least 85%, at least 90%, at least 95%, at least 96%, at least 97%, at least 98%, or at least 99% identical to SEQ ID NO: 172, a construct having a sequence which is at least 70%, at least 75%, at least 80%, at least 85%, at least 90%, at least 95%, at least 96%, at least 97%, at least 98%, or at least 99% identical to a sequence selected from the group consisting of SEQ ID NOS:122-169, and a sequence which is at least 70%, at least 75%, at least 80%, at least 85%, at least 90%, at least 95%, at least 96%, at least 97%, at least 98%, or at least 99% identical to SEQ ID NO:173. In some embodiments, the self- replicating RNA molecule comprises from 5′ to 3′ a sequence that is a fragment of SEQ ID NO: 172, a fragment of a full-length construct sequence selected from the group consisting of SEQ ID NOS: 122-169, and a sequence that is a fragment of SEQ ID NO: 173, wherein a fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, a DNA sequence encoding a self-replicating RNA molecule is provided, the DNA sequence selected from SEQ ID NO:177 or SEQ ID NO:179. In some embodiments, the DNA sequence encoding a self-replicating RNA molecule comprises a sequence which is at least 70%, at least 75%, at least 80%, at least 85%, at least 90%, at least 95%, at least 96%, at least 97%, at least 98%, or at least 99% identical to a sequence selected from any one of SEQ ID NO: 177 or SEQ ID NO: 179. In some embodiments, the DNA sequence encoding a self-replicating RNA molecule comprises a fragment of a full-length sequence selected from any one of SEQ ID NO: 177 or SEQ ID NO:179 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, a DNA sequence encoding a self-replicating RNA molecule is provided, said DNA sequence comprising from 5′ to 3′ a DNA sequence having SEQ ID NO:174, a DNA sequence having a sequence selected from the group consisting of SEQ ID NOS:74-121, and a DNA sequence having SEQ ID NO:175. In some embodiments, a DNA sequence encoding a self-replicating RNA molecule is provided, said DNA sequence comprising from 5′ to 3′ a sequence which is at least 70%, at least 75%, at least 80%, at least 85%, at least 90%, at least 95%, at least 96%, at least 97%, at least 98%, or at least 99% identical to SEQ ID NO:174, a DNA sequence which is at least 70%, at least 75%, at least 80%, at least 85%, at least 90%, at least 95%, at least 96%, at least 97%, at least 98%, or at least 99% identical to a sequence selected from the group consisting of SEQ ID NOS:74-121, and a DNA sequence which is at least 70%, at least 75%, at least 80%, at least 85%, at least 90%, at least 95%, at least 96%, at least 97%, at least 98%, or at least 99% identical to SEQ ID NO: 175. In some embodiments, the DNA sequence encoding a self-replicating RNA molecule comprises from 5′ to 3′ a sequence that is a fragment of SEQ ID NO:174, a fragment of a full-length construct sequence selected from the group consisting of SEQ ID NOS:74-121, and a sequence that is a fragment of SEQ ID NO:175, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

The nucleic acid-based vaccine may comprise a viral or a non-viral delivery system. The delivery system (also referred to herein as a delivery vehicle) may have adjuvant effects which enhance the immunogenicity of the encoded Coronavirus S antigen. For example, the nucleic acid molecule may be encapsulated in liposomes, non-toxic biodegradable polymeric microparticles or viral replicon particles (VRPs), or complexed with particles of a cationic oil-in-water emulsion. In some embodiments, the nucleic acid-based vaccine comprises a cationic nano-emulsion (CNE) delivery system or a lipid nanoparticle (LNP) delivery system. In some embodiments, the nucleic acid-based vaccine comprises a non-viral delivery system, i.e., the nucleic acid-based vaccine is substantially free of viral capsid. Alternatively, the nucleic acid-based vaccine may comprise viral replicon particles. In other embodiments, the nucleic acid-based vaccine may comprise a naked nucleic acid, such as naked RNA (e.g. mRNA), but delivery via CNEs or LNPs is preferred.

In certain embodiments, the nucleic acid-based vaccine comprises a cationic nano-emulsion (CNE) delivery system. CNE delivery systems and methods for their preparation are described in the following reference: WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in the following references: WO2012/006380; WO2013/006834; and WO2013/006837 (the contents of each of which are incorporated herein in their entirety).

Thus, in a nucleic acid-based vaccine of the invention, an RNA molecule encoding a Coronavirus S antigen may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non- viral delivery system, such as CNE, and the nucleic acid-based vaccine comprises a self-replicating RNA (mRNA). This may be particularly effective in eliciting humoral and cellular immune responses. Advantages also include the absence of a limiting anti-vector immune response and a lack of risk of genomic integration.

LNP delivery systems and non-toxic biodegradable polymeric microparticles, and methods for their preparation are described in the following references: WO2012/006376 (LNP and microparticle delivery systems); Geall et al. (2012) PNAS USA. Sep 4; 109(36): 14604-9 (LNP delivery system); and WO2012/006359 (microparticle delivery systems). LNPs are non- virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. Sep 4; 109(36): 14604-9.

### Pharmaceutical Compositions; Immunogenic Compositions

The disclosure provides compositions comprising a nucleic acid comprising a sequence which encodes a Coronavirus polypeptide, for example a Coronavirus S antigen. The composition may be a pharmaceutical composition, e.g., an immunogenic composition or a vaccine composition. Accordingly, the composition may also comprise a pharmaceutically acceptable carrier. In some embodiments, the Coronavirus is SARS CoV-2.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier.

Pharmaceutical compositions may include the constructs, nucleic acid sequences, and/or polypeptide sequences described elsewhere herein in plain water (e.g. “w.f.i.”) or in a buffer e.g. a phosphate buffer, a Tris buffer, a borate buffer, a succinate buffer, a histidine buffer, or a citrate buffer. Buffer salts will typically be included in the 5-20 mM range. Pharmaceutical compositions may have a pH between 5.0 and 9.5 e.g. between 6.0 and 8.0. Compositions may include sodium salts (e.g. sodium chloride) to give tonicity. A concentration of 10±2 mg/mL NaCl is typical, e.g. about 9 mg/mL. Compositions may include metal ion chelators. These can prolong RNA stability by removing ions which can accelerate phosphodiester hydrolysis. Thus a composition may include one or more of EDTA, EGTA, BAPTA, pentetic acid, etc.. Such chelators are typically present at between 10-500 µM e.g. 0.1 mM. A citrate salt, such as sodium citrate, can also act as a chelator, while advantageously also providing buffering activity. Pharmaceutical compositions may have an osmolality of between 200 mOsm/kg and 400 mOsm/kg, e.g. between 240-360 mOsm/kg, or between 290-310 mOsm/kg. Pharmaceutical compositions may include one or more preservatives, such as thiomersal or 2-phenoxyethanol. Mercury-free compositions are preferred, and preservative-free vaccines can be prepared. Pharmaceutical compositions may be aseptic or sterile. Pharmaceutical compositions may be non-pyrogenic e.g. containing <1 EU (endotoxin unit, a standard measure) per dose, and preferably <0.1 EU per dose. Pharmaceutical compositions may be gluten free. Pharmaceutical compositions may be prepared in unit dose form. In some embodiments a unit dose may have a volume of between 0.1 -1.0 mL e.g. about 0.5 mL.

In some embodiments, the compositions disclosed herein are immunogenic composition that, when administered to a subject, induce a humoral and/or cellular antigen-specific immune response (i.e. an immune response which specifically recognizes a naturally occurring Coronavirus polypeptide). For example, an immunogenic composition may induce a memory T and/or B cell population relative to an untreated subject following Coronavirus infection, particularly in those embodiments where the composition comprises a nucleic acid comprising a sequence which encodes a Coronavirus S antigen or comprises a Coronavirus antigen. In some embodiments, the subject is a vertebrate, such as a mammal e.g. a human or a veterinary mammal.

The compositions of the invention can be formulated as vaccine compositions. The vaccine will comprise an immunologically effective amount of antigen. By “an immunologically effective amount” is intended that the administration of that amount to a subject, either in a single dose or as part of a series, is effective for inducing a measurable immune response against Coronavirus in the subject. This amount varies depending upon the health and physical condition of the individual to be treated, age, the taxonomic group of individual to be treated (e.g. human, non-human primate, etc.), the capacity of the individual’s immune system to synthesize antibodies, the degree of protection desired, the formulation of the composition or vaccine, the treating doctor’s assessment of the medical situation, the severity of the disease, the potency of the compound administered, the mode of administration, and other relevant factors. It is expected that the amount will fall in a relatively broad range that can be determined through routine trials. Vaccines as disclosed herein may either be prophylactic (i.e. to prevent infection) or therapeutic (i.e. to treat infection), but will typically be prophylactic. In some embodiments, the vaccine compositions disclosed herein may induce an effective immune response against a Coronavirus infection, i.e., a response sufficient for treatment or prevention of a Coronavirus infection.

### Methods of Use/Uses

In some embodiments are provided methods for inducing an immune response against a Coronavirus infection in a subject in need thereof comprising a step of administering an immunologically effective amount of a construct or composition as disclosed herein. In some embodiments are provided the use of the constructs or compositions disclosed herein for inducing an immune response to a Coronavirus S antigen in a subject in need thereof. In some embodiments are provided the use of the constructs or compositions disclosed herein for inducing an immune response against a Coronavirus infection in a subject. In some embodiments are provided use of the construct or composition as disclosed herein in the manufacture of a medicament inducing an immune response to a Coronavirus infection in a subject. In some embodiments are provided the use of the constructs or compositions disclosed herein for inducing an immune response against a SARS CoV-2 infection in a subject. In some embodiments are provided use of the construct or composition as disclosed herein in the manufacture of a medicament inducing an immune response to a SARS CoV-2 infection in a subject. By “subject” is intended a vertebrate, such as a mammal e.g. a human or a veterinary mammal. In some embodiments the subject is human. In some embodiments the composition comprises an RNA molecule encoding a polypeptide selected from the group consisting of SEQ ID NOs:26-73. In some embodiments, the composition comprises an RNA molecule encoding a polypeptide which is at least 70%, at least 75%, at least 80%, at least 85%, at least 90%, at least 95%, at least 96%, at least 97%, at least 98%, or at least 99% identical to a sequence selected from the group consisting of SEQ ID NOS:26-73. In some embodiments, the composition comprises an RNA molecule encoding a polypeptide which comprises a fragment of a full-length sequence selected from the group consisting of SEQ ID NOs:26-73, wherein the fragment comprises a contiguous stretch of the amino acid sequence of the full-length sequence up to 1, 2, 3, 4, 5, 6, 7, 8, 9, or 10 amino acids shorter than full-length sequence.

### Routes of Administration/Dosages

Compositions disclosed herein will generally be administered directly to a subject. Direct delivery may be accomplished by parenteral injection, typically intramuscularly.

A dose of a nucleic acid (e.g. a nucleic acid-based vaccine) may have <10(µg nucleic acid; e.g. from 0.001-10 µg, such as about 1 µg, 2.5 µg, 5 µg, 7.5 µg or 10 µg, but expression can be seen at much lower levels; e.g. using <1 µg/dose, <100 ng/dose, <10 ng/dose, <1 ng/dose, etc. Similarly, a dose of a protein antigen may have <10 µg protein; e.g. from 1-10 µg, such as about 1 µg, 2.5 µg, 5 µg, 7.5 µg or 10 µg.

### Processes of Manufacture/Formulation

Processes for the manufacture of self-replicating RNA are provided herein. In some embodiments, the process of manufacturing a self-replicating RNA comprises a step of in vitro transcription (IVT) as described elsewhere herein. In some embodiments, the process of manufacturing a self-replicating RNA comprises a step of IVT to produce a RNA, and further comprises a step of combining the RNA with a non-viral delivery system as described elsewhere herein. In some embodiments, the process of manufacturing a self-replicating RNA comprises a step of IVT to produce a RNA, and further comprises a step of combining the RNA with a CNE delivery system as described elsewhere herein. In some embodiments, the process of manufacturing a self-replicating RNA comprises IVT to produce a RNA, and further comprises combining the RNA with a LNP delivery system as described herein.

### Sequence Identity

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

Where the present disclosure refers to a sequence by reference to a UniProt or Genbank accession code, the sequence referred to is the current version at the filing date of the present application.

### Part A, Embodiments of the Invention

Part A, Embodiment 1. A construct comprising a nucleic acid sequence selected from the group consisting of:
(a) a nucleic acid sequence encoding a polypeptide comprising the amino acid sequence of SEQ ID NO:49 and SEQ ID NO:26;
(b) a nucleic acid sequence comprising the DNA sequence of SEQ ID NO:97 and SEQ ID NO:74;
(c) a nucleic acid sequence comprising the RNA sequence of SEQ ID NO:145 and SEQ ID NO:122; and
(d) a variant or fragment of (a)-(c).

Part A, Embodiment 2. The construct of Part A, Embodiment 1, wherein the nucleic acid is a RNA molecule.

Part A, Embodiment 3. The construct of Part A, Embodiments 1-2, wherein the RNA is a self-replicating RNA molecule.

Part A, Embodiment 4. A self-replicating RNA molecule comprising a construct encoding a polypeptide comprising a Coronavirus spike (S) antigen, or an immunogenic variant or fragment thereof.

Part A, Embodiment 5. The construct of Part A, Embodiment 4, wherein the construct encodes a prefusion stabilized SARS CoV-2 S antigen, or an immunogenic variant or fragment thereof.

Part A, Embodiment 6. The construct of any one of Part A, Embodiments 4-5, wherein the construct encodes a prefusion stabilized Coronavirus S antigen, or an immunogenic variant or fragment thereof.

Part A, Embodiment 7. The construct of Part A, Embodiment 6, further comprising proline substitutions at residues 986 and 987 of SEQ ID NO: 1.

Part A, Embodiment 8. The construct of any of Part A, Embodiments 6-7, further comprising a GSAS (SEQ ID NO:190) substitution at the furin cleavage site (residues 682-685 of SEQ ID NO: 1).

Part A, Embodiment 9. The construct of any of Part A, Embodiments 4-8, further comprising a C-terminal transmembrane sequence.

Part A, Embodiment 10. The construct of any of Part A, Embodiments 4-8, further comprising a sequence C-terminal T4 fibritin trimerization (foldon) motif.

Part A, Embodiment 11. A self-replicating RNA molecule comprising a nucleic acid sequence selected from the group consisting of SEQ ID NO:4 and SEQ ID NO:9.

Part A, Embodiment 12. A DNA molecule encoding the self-replicating RNA molecule of Part A, Embodiments 3-11.

Part A, Embodiment 13. The DNA molecule of Part A, Embodiment 12 comprising a nucleic acid sequence selected from the group consisting of SEQ ID NO:5 and SEQ ID NO:10.

Part A, Embodiment 14. A composition comprising an immunologically effective amount of one or more of the constructs of any of Part A, Embodiments 1-2 or the self-replicating RNA molecules of any of Part A, Embodiments 3-11.

Embodiment 15. The composition according to Part A, Embodiment 14 comprising the self-replicating RNA molecule of embodiment 11.

Part A, Embodiment 16. The composition according to any of embodiments 14-15, wherein the composition comprises a non-viral delivery material, such as a submicron cationic oil-in-water emulsion; a liposome; or a biodegradable polymeric microparticle delivery system.

Part A, Embodiment 17. The composition according to embodiment 16, wherein the composition comprises a submicron cationic oil-in-water emulsion.

Part A, Embodiment 18. The composition according to embodiment 16, wherein the composition comprises a liposome.

Part A, Embodiment 19. The composition according to any of embodiments 14-18 wherein the composition further comprises a nucleic acid sequence which encodes an additional antigen.

Part A, Embodiment 20. The composition according to any of embodiments 14-19 wherein the composition further comprises a self-replicating RNA which encodes an additional antigen.

Part A, Embodiment 21. The composition according to any of embodiments 14-20 wherein the composition is pharmaceutically acceptable for administration to a subject by intramuscular injection.

Part A, Embodiment 22. A method of inducing an immune response against a Coronavirus infection in a subject in need thereof, which comprises administering to said subject an immunologically effective amount of the composition of embodiments 14-21.

Part A, Embodiment 23. The method according to embodiment 22 wherein the immune response is characterized by immunological memory against the Coronavirus and/or an effective Coronavirus-responsive memory T cell population.

Part A, Embodiment 24. The method according to any of embodiments 22-23 wherein the subject is human.

Part A, Embodiment 25. A process for producing an RNA-based vaccine comprising a step of transcribing the DNA of embodiment 13 to produce an RNA comprising a coding region for the antigen.

Part A, Embodiment 26. The process of embodiment 25, wherein said transcription is in vitro.

Part A, Embodiment 27. The process of embodiments 25, wherein said transcription is in vivo.

Part A, Embodiment 28. The process of any embodiment 25-27, further comprising a step of formulating the RNA comprising the coding region for the antigen with a delivery system.

Part A, Embodiment 29. The process of embodiment 28, wherein the delivery system is a non-viral delivery material.

Part A, Embodiment 30. The process of embodiment 29, wherein the delivery system is a submicron cationic oil-in-water emulsion.

Part A, Embodiment 31. The process of any of embodiments 25-30, wherein the delivery system is a liposome.

Part A, Embodiment 32. The process of embodiment 31, wherein said liposome comprises a lipid comprising a tertiary amine.

Part A, Embodiment 33. A composition produced by the process of any of embodiments 25-32.

Part A, Embodiment 34. Use of the self-replicating RNA of embodiments 1-8; the construct of embodiment 9; the RNA molecule of embodiment 10; the self-replicating RNA molecule of embodiments 11-12; or a composition of any one of embodiments 14-21 for inducing an immune response to a Coronavirus infection in a subject.

Part A, Embodiment 35. Use of the construct of embodiments 1-9; the vector of embodiment 10; the self-replicating RNA molecule of embodiments 11-12; or a composition of any one of embodiments 14-21 in the manufacture of a medicament inducing an immune response against a Coronavirus infection in a subject.

Part A, Embodiment 36. Use of the self-replicating RNA of embodiments 1-8; the construct of embodiment 9; the RNA molecule of embodiment 10; the self-replicating RNA molecule of embodiments 11-12; or a composition of any one of embodiments 14-21 for inducing an immune response to a SARS CoV-2 infection in a subject.

Part A, Embodiment 35. Use of the construct of embodiments 1-9; the vector of embodiment 10; the self-replicating RNA molecule of embodiments 11-12; or a composition of any one of embodiments 14-21 in the manufacture of a medicament inducing an immune response against a SARS CoV-2 infection in a subject.

### Part B, Embodiments of the Invention

Part B, Embodiment 1. A construct comprising a nucleic acid sequence encoding an antigen, wherein the antigen comprises a heterologous signal sequence, a mutation with respect to the wild-type sequence which affects retention of the antigen in the endoplasmic reticulum (ER), or a combination thereof.

Part B, Embodiment 2. The construct of Part B, Embodiment 1, wherein said antigen is a coronavirus antigen, preferably a coronavirus S protein.

Part B, Embodiment 3. The construct of Part B, Embodiment 2, wherein said coronavirus S protein is a SARS CoV-2 S protein.

Part B, Embodiment 4. The construct of Part B, Embodiment 3, wherein the heterologous signal sequence replaces residues 1-16 of the SARS CoV-2 S protein as shown in SEQ ID NO:1, or corresponding residues in another SARS CoV-2 S protein sequence.

Part B, Embodiment 5. The construct of Part B, Embodiment 3, wherein the heterologous signal sequence replaces residues 1-18 of the SARS CoV-2 S protein as shown in SEQ ID NO:1, or corresponding residues in another SARS CoV-2 S protein sequence.

Part B, Embodiment 6. The construct of any one of Part B, Embodiments 1 to 5, wherein the heterologous signal sequence has a sequence selected from:
a) a Gaussian Luciferase signal sequence as shown in SEQ ID NO:2,
b) a human CD5 signal sequence as shown in SEQ ID NO:3,
c) a human CD33 signal sequence as shown in SEQ ID NO:4,
d) a human IL2 signal sequence as shown in SEQ ID NO:5,
e) a human IgE signal sequence as shown in SEQ ID NO:6,
f) a human Light Chain Kappa signal sequence as shown in SEQ ID NO:7,
g) a JEV short signal sequence as shown in SEQ ID NO:8,
h) a JEV long signal sequence as shown in SEQ ID NO:9,
i) a Mouse Light Chain Kappa signal sequence as shown in SEQ ID NO:10,
j) a SSP signal sequence as shown in SEQ ID NO:11,
k) a Gaussian Luciferase (AKP) signal sequence as shown in SEQ ID NO:12, and
l) a variant of any one of sequences (a)-(k) having 1, 2, 3, 4 or 5 amino acid residue deletions, insertions or substitutions.

Part B, Embodiment 7. The construct of any one of Part B, Embodiments 1 to 6, wherein the antigen is a SARS CoV-2 S protein, and wherein said mutation with respect to the wild-type sequence is selected from:
a) the substitution of residues K1269 and H1271 as shown in SEQ ID NO:1 to alanine residues, or corresponding substitutions in another SARS Cov-2 S protein sequence, and
b) the deletion of residues 1261-1273 of SEQ ID NO:1, or of corresponding residues in another SARS Cov-2 S protein sequence.

Part B, Embodiment 8. The construct of any one of Part B, Embodiments 1 to 7, wherein the antigen is a coronavirus S protein and comprises one or more mutations with respect to the wild-type sequence which stabilize the prefusion form of the coronavirus S protein.

Part B, Embodiment 9. The construct of Part B, Embodiment 8, wherein the coronavirus S protein is a SARS CoV-2 S protein, and wherein said one or more mutations comprise the substitutions of residues ⁹⁸⁶KV⁹⁸⁷ as shown in SEQ ID NO:1 to ⁹⁸⁸PP⁹⁸⁷, and/or the substitution of residues ⁶⁸²RRAR⁶⁸⁵ (SEQ ID NO:188) as shown in SEQ ID NO:1 to ⁶⁸²GSAS⁶⁸⁵ (SEQ ID NO:190) or corresponding mutations in another SARS Cov-2 S protein sequence.

Part B, Embodiment 10. The construct of any one of Part B, Embodiments 1 to 9, wherein the antigen is a coronavirus S protein, and wherein the native transmembrane and cytosolic domains of the coronavirus S protein are replaced by a heterologous trimerization domain, preferably a C-terminal T4 fibritin trimerization (foldon) motif.

Part B, Embodiment 11. The construct of Part B, Embodiment 10, wherein said the coronavirus S protein is a SARS CoV-2 S protein, and wherein residues 1208-1273 of the sequence shown in SEQ ID NO:1, or corresponding mutations in another SARS Cov-2 S protein sequence, are replaced by a C-terminal T4 fibritin trimerization (foldon) motif having the sequence shown in SEQ ID NO:24.

Part B, Embodiment 12. The construct of any one of Part B, Embodiments 1 to 11, wherein the antigen is a SARS CoV-2 S protein, and wherein said S protein:
a) has an amino acid sequence selected from SEQ ID NOs:27-73, or a variant which is at least 90% identical thereto;
b) is encoded by a DNA sequence having a sequence selected from SEQ ID NOs:75-121, or a variant which is at least 90% identical thereto; or
c) is encoded by an RNA sequence having a sequence selected from SEQ ID NOs:123-169, or a variant which is at least 90% identical thereto.

Part B, Embodiment 13. The construct of any one of Part B, Embodiments 1 to 12, wherein said nucleic acid is an RNA molecule.

Part B, Embodiment 14. A self-replicating RNA comprising the construct of Part B, Embodiment 13.

Part B, Embodiment 15. The self-replicating RNA of Part B, Embodiment 14 comprising the elements of a VEE TC-83 replicon including viral nonstructural proteins 1-4 (nsP1-4), followed by a subgenomic promoter, and a construct encoding the antigen.

Part B, Embodiment 16. The self-replicating RNA of Part B, Embodiment 15, wherein the VEE TC-83 replicon has the sequence shown in SEQ ID NO:171, and the construct encoding the S coronavirus protein is inserted immediately after residue 7561.

Part B, Embodiment 17. A self-replicating RNA comprising from 5′ to 3′ a sequence having SEQ ID NO: 172, a construct having a sequence selected from the group consisting of SEQ ID NOS:122-169, and a sequence having SEQ ID NO:173.

Part B, Embodiment 18. A DNA molecule encoding the self-replicating RNA of any one of Part B, Embodiments 14 to 17.

Part B, Embodiment 19. A composition comprising an immunologically effective amount of one or more of the constructs of any of one of Part B, Embodiments 1 to 13, the self-replicating RNA of any one of Part B, Embodiments 14 to 17 or the DNA molecule of Part B, Embodiment 18.

Part B, Embodiment 20. The composition of Part B, Embodiment 19, wherein the composition comprises a non-viral delivery material, such as a submicron cationic oil-in-water emulsion; a liposome; or a biodegradable polymeric microparticle delivery system.

Part B, Embodiment 21. The composition of Part B, Embodiment 20, wherein the composition comprises a submicron cationic oil-in-water emulsion.

Part B, Embodiment 22. The composition according to Part B, Embodiment 20, wherein the composition comprises a liposome.

Part B, Embodiment 23. The composition of any of Part B, Embodiments 19 to 22 wherein the composition further comprises a nucleic acid sequence which encodes an additional antigen.

Part B, Embodiment 24. The composition of any of Part B, Embodiments 19 to 22 wherein the composition further comprises a self-replicating RNA which encodes an additional antigen.

Part B, Embodiment 25. The composition of any of Part B, Embodiments 19 to 24 wherein the composition is pharmaceutically acceptable for administration to a subject by intramuscular injection.

Part B, Embodiment 26. A process for producing an RNA-based vaccine comprising a step of transcribing the DNA molecule of Part B, Embodiment 18 to produce a self-replicating RNA comprising a coding region for the coronavirus S protein.

Part B, Embodiment 27. The process of Part B, Embodiment 26, wherein said transcription is in vitro.

Part B, Embodiment 28. The process of Part B, Embodiments 26, wherein said transcription is in vivo.

Part B, Embodiment 29. The process of Part B, Embodiment 26, 27 or 28, further comprising a step of formulating the self-replicating RNA comprising the coding region for the coronavirus S protein with a delivery system.

Part B, Embodiment 30. The process of Part B, Embodiment 29, wherein the delivery system is a non-viral delivery material.

Part B, Embodiment 31. The process of Part B, Embodiment 30, wherein the delivery system is a submicron cationic oil-in-water emulsion.

Part B, Embodiment 32. The process of Part B, Embodiment 30, wherein the delivery system is a liposome.

Part B, Embodiment 33. The process of Part B, Embodiment 32, wherein said liposome comprises a lipid comprising a tertiary amine.

Part B, Embodiment 34. A composition produced by the process of any one of Part B, Embodiments 26 to 33.

Part B, Embodiment 35. A method of inducing an immune response against a Coronavirus infection in a subject in need thereof, which comprises administering to said subject an immunologically effective amount of the construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34.

Part B, Embodiment 36. The method of Part B, Embodiment 35 wherein the immune response is characterized by immunological memory against the Coronavirus and/or an effective Coronavirus-responsive memory T cell population.

Part B, Embodiment 37. The method of Part B, Embodiment 26 or 27 wherein the subject is human.

Part B, Embodiment 38. The construct of any one of Part B, Embodiments 1 to 13, the self-replicating RNA of any one of Part B, Embodiments 13 to 17, the DNA molecule of Part B, Embodiment 18, or the composition of any one of Part B, Embodiments 19 to 25 or 34, for use in therapy.

Part B, Embodiment 39. The construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34, for use in preventing or treating a coronavirus infection in a subject.

Part B, Embodiment 40. The construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34, for use in preventing or treating a SARS CoV-2 infection in a subject.

Part B, Embodiment 41. Use of the construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34 for inducing an immune response to a Coronavirus infection in a subject.

Part B, Embodiment 42. Use of the construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34 in the manufacture of a medicament inducing an immune response against a Coronavirus infection in a subject.

Part B, Embodiment 43. Use of the construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34 for inducing an immune response to a SARS CoV-2 infection in a subject.

Part B, Embodiment 44. Use of the construct of any one of Part B, Embodiments 2 to 13, the self-replicating RNA of any one of Part B, Embodiments 13 to 17, the DNA molecule of Part B, Embodiment 18, or the composition of any one of Part B, Embodiments 19 to 25 or 34 in the manufacture of a medicament inducing an immune response against a SARS CoV-2 infection in a subject.

### General

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The invention will be further described by reference to the following, non-limiting, figures and examples.

### EXAMPLES

### Example 1. Project Summary

The present inventors initiated work on a Coronavirus vaccine using the SAM platform - synthetic, self-amplifying mRNA derived from the alphavirus genome, expressing antigens of interest. The SAM constructs are evaluated for robust antigen production and antigenicity and further tested for their immunogenicity and efficacy using in vivo models.

### Methods

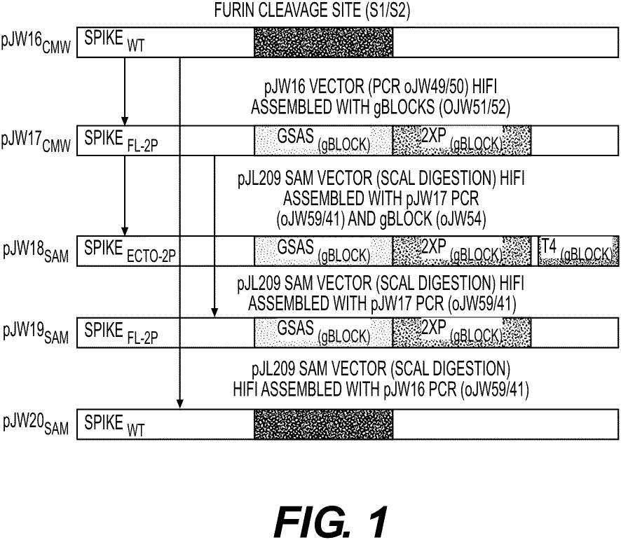

The SAM vector VEE TC-83 (SEQ ID NO:171, FIG. 1)was used as the background construct for cloning in the Examples. The empty vector is shown in SEQ ID NO:171; the insert encoding the SARS-CoV2 S antigen starts immediately after nucleotide 7561. The sequence of the DNA plasmid encoding the SAM VEE TC-83 is shown in FIG. 8 (SEQ ID NO:170).

### Example 2. Design of SARS-CoV2 S Antigen Constructs

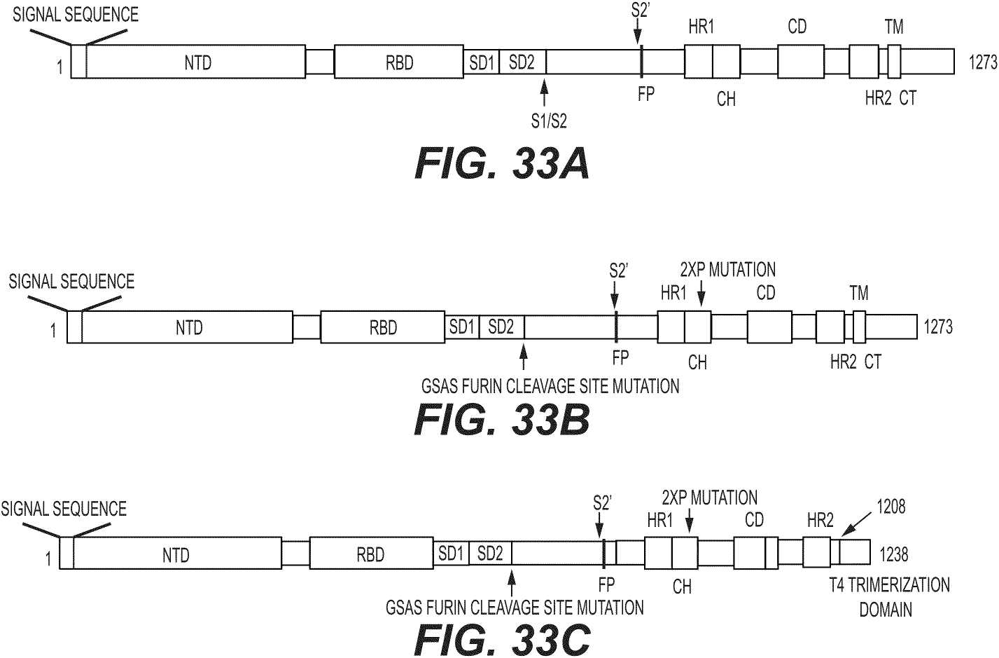

The inventors designed full length and ecto domain SARS-CoV2 S antigens. The structure of the wild-type 2019-nCoV S protein (SEQ ID NO:1) is shown in FIG. 33A. All constructs contained proline substitutions K986P and V987P to help stabilize the prefusion form of the S protein and a “GSAS” (SEQ ID NO:190) substitution (residues 682-685) in the furin cleavage site Wrapp et al, 2020, Science) (FIG. 33B, FIG. 33C). In all ecto domain constructs, the entire C-terminus (including the transmembrane domain) after amino acid 1208 was removed and replaced by a T4 fibritin trimerization motif (foldon) in order to promote the formation of trimeric complexes (FIG. 33C). The amino acid sequence of the T4 fibritin trimerization motif (foldon) is shown in SEQ ID NO:24, and its DNA sequence is shown in SEQ ID NO:25.

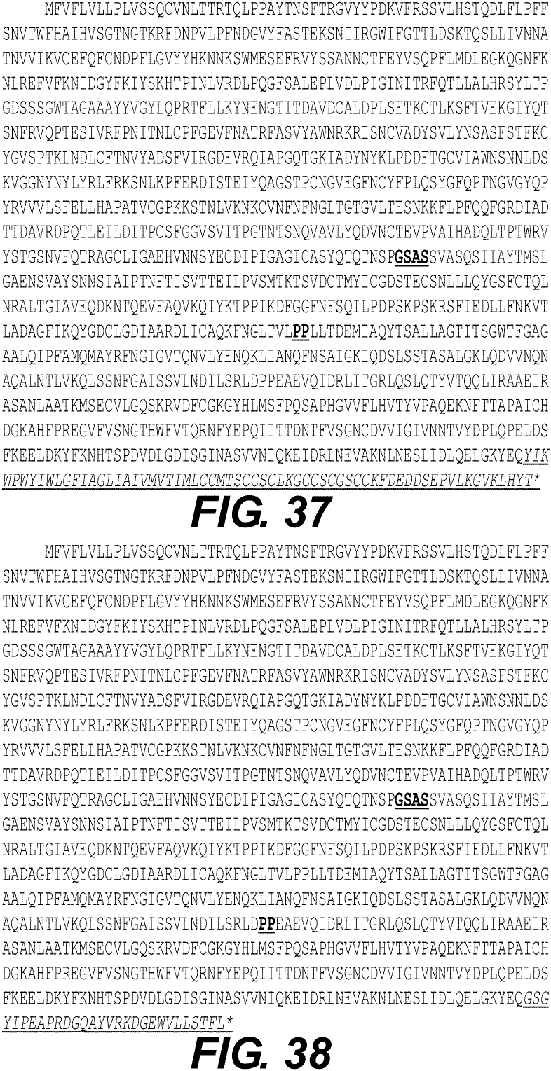

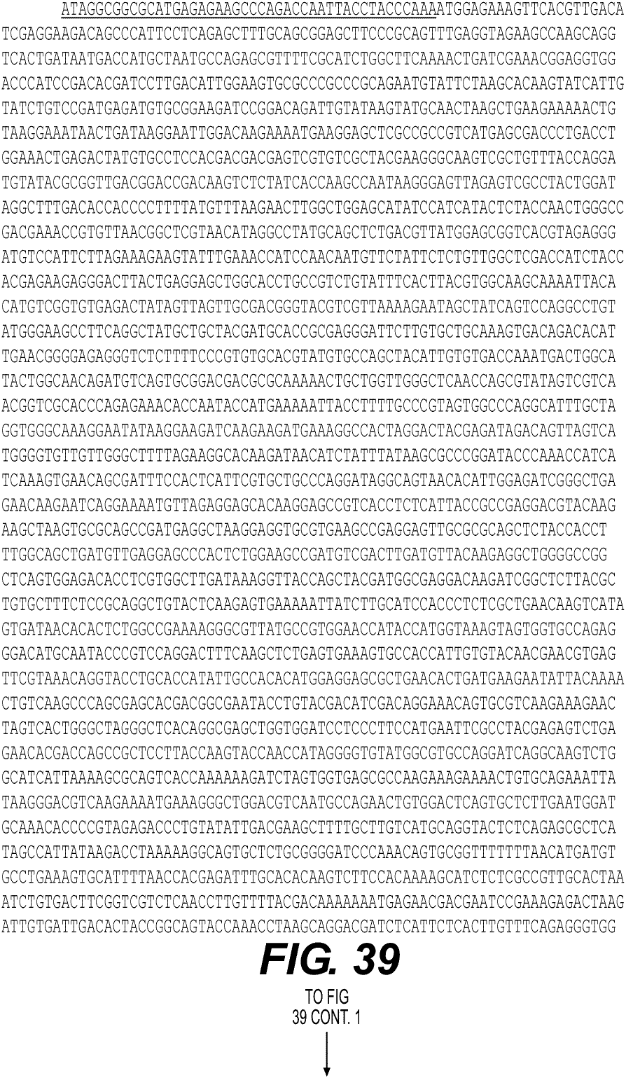

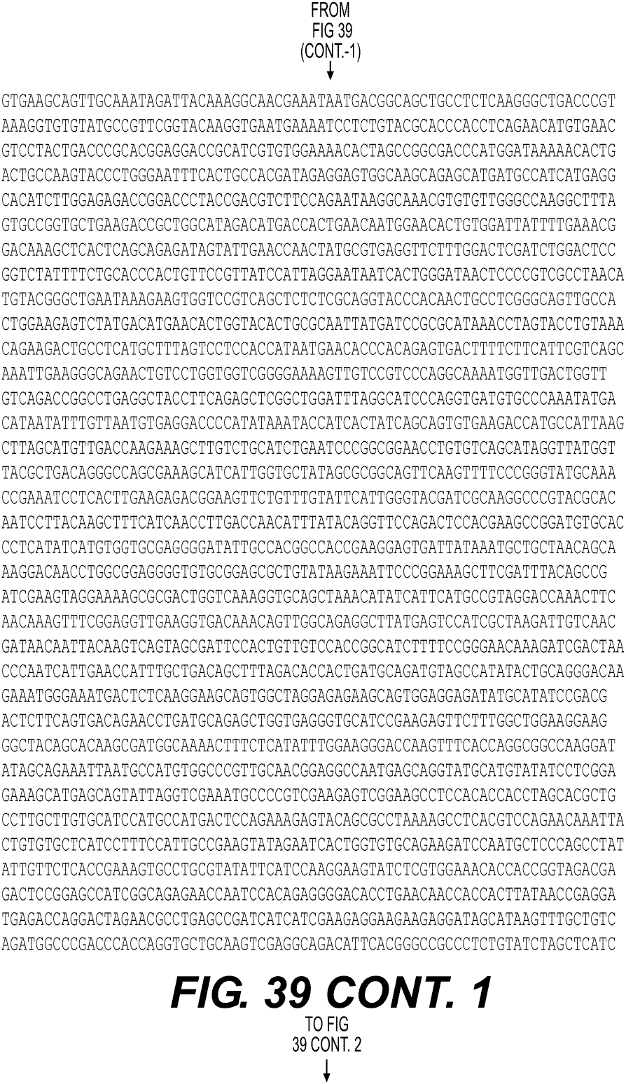

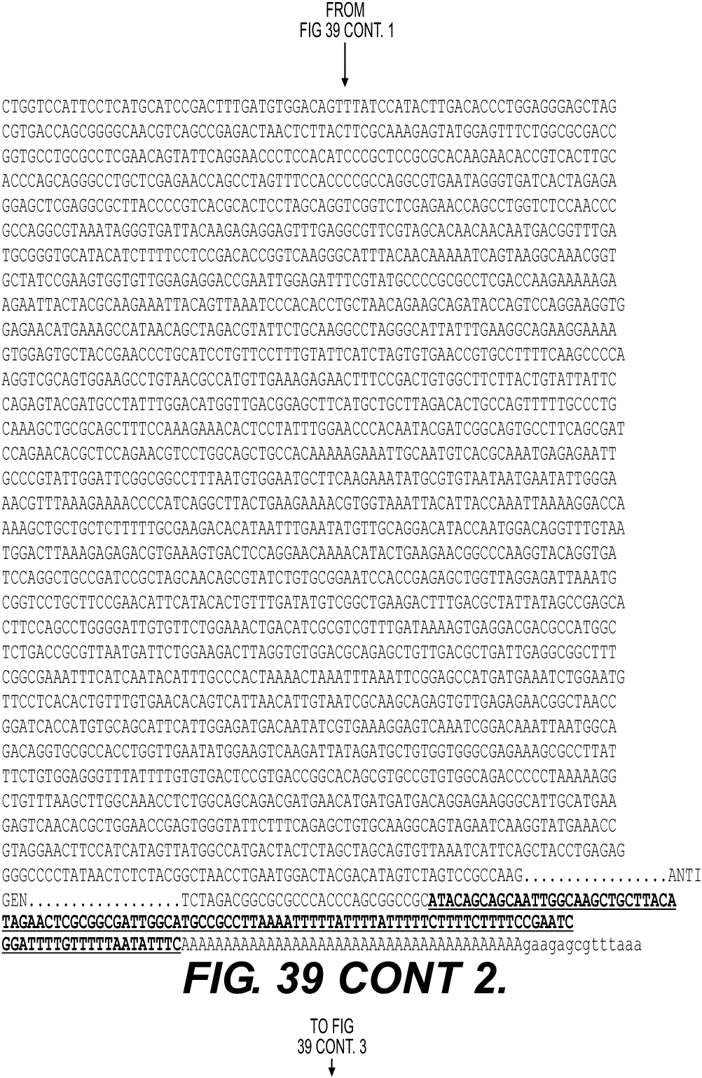

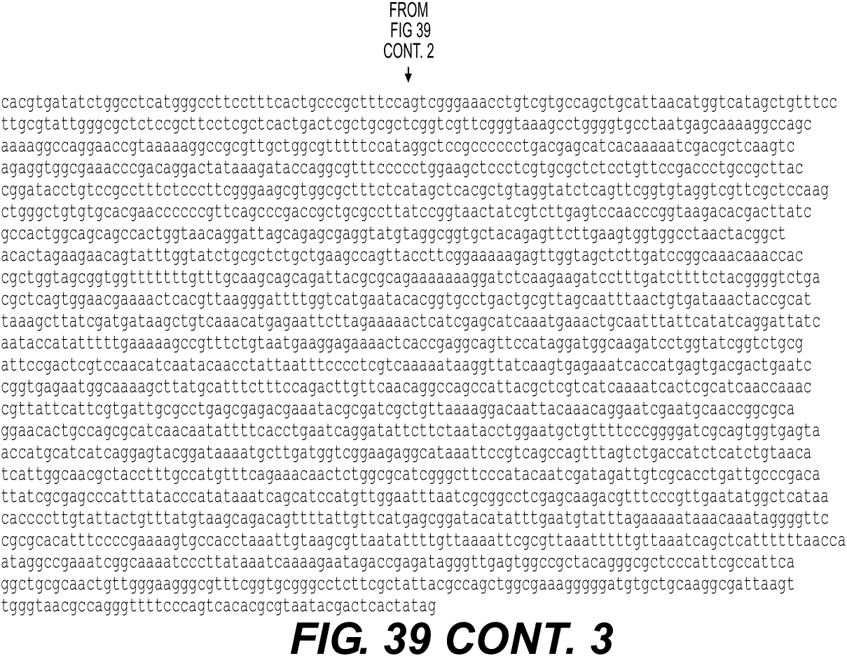

The first design of SAM Coronavirus constructs (constructs A and B of FIGS. 37 and 38) includes cloning the sequence encoding the Coronavirus S protein under the subgenomic promoter in a SAM vector. A series of modifications to the S protein were made (Inset 1). In addition, the wild type sequence was modified by:
i. Codon optimization of the coding sequence for the antigen; and
ii. Removal of several restriction sites.

Inset 1

SAM constructs

| | |
|---|---|
| Construct | Description |
| A | • proline substitutions at residues 986 and 987, |
| | • a “GSAS” (SEQ ID NO:190) substitution at the furin cleavage site (residues 682-685) |
| | • protein native sequence for transmembrane and C terminal domains |
| B | • proline substitutions at residues 986 and 987, |
| | • a “GSAS” (SEQ ID NO:190) substitution at the furin cleavage site (residues 682-685) |
| | • C-terminal T4 fibritin trimerization motif (foldon) |

Construct A is described by sequence identifier in Table 3; construct B is described by sequence identifier in Table 2.

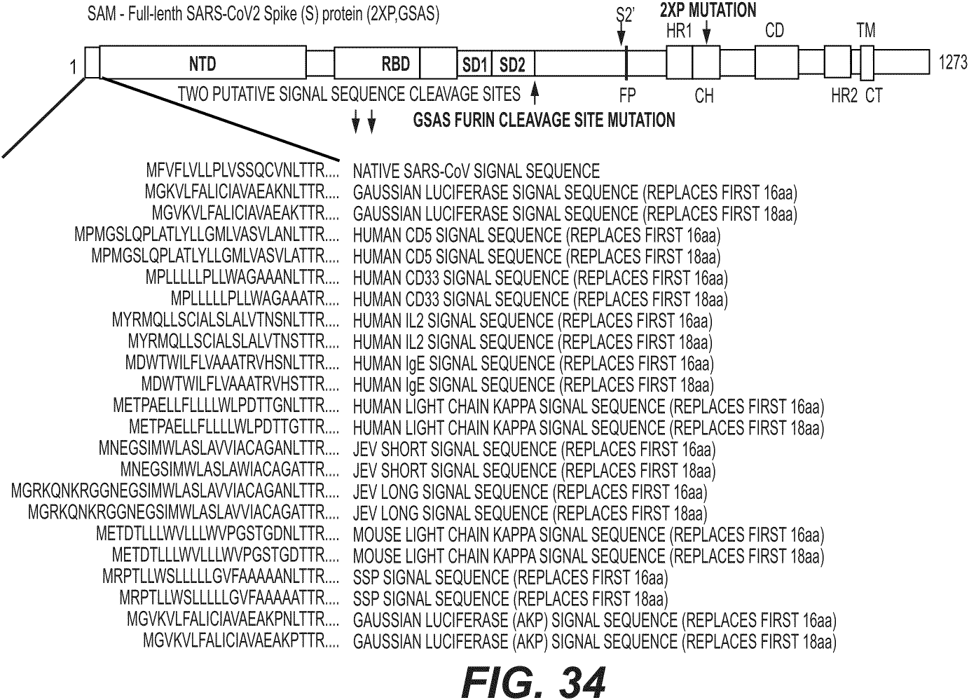

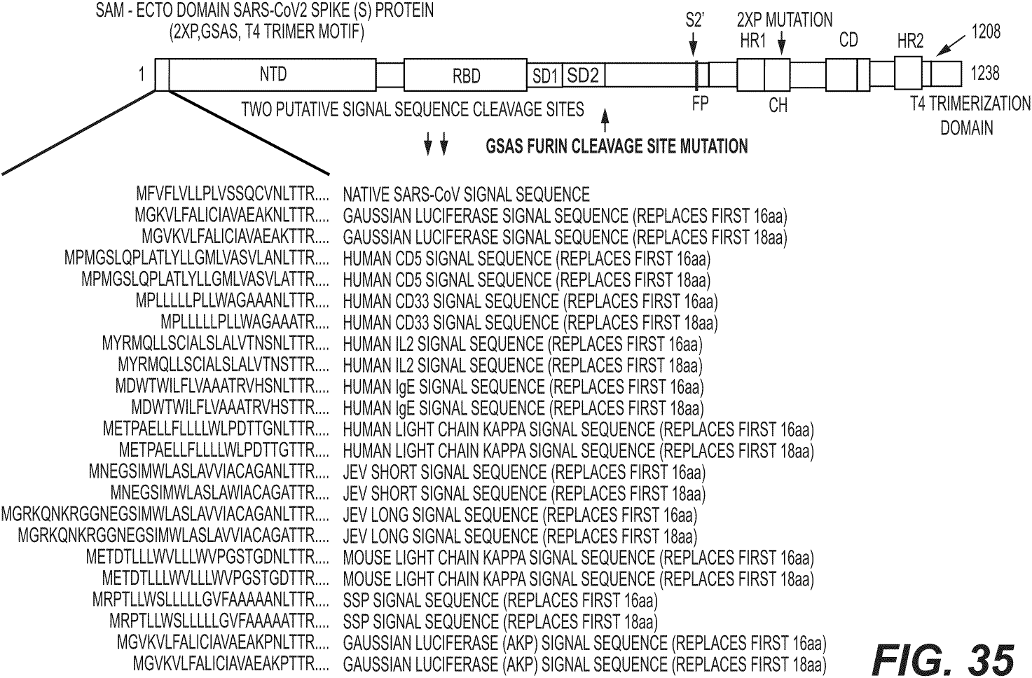

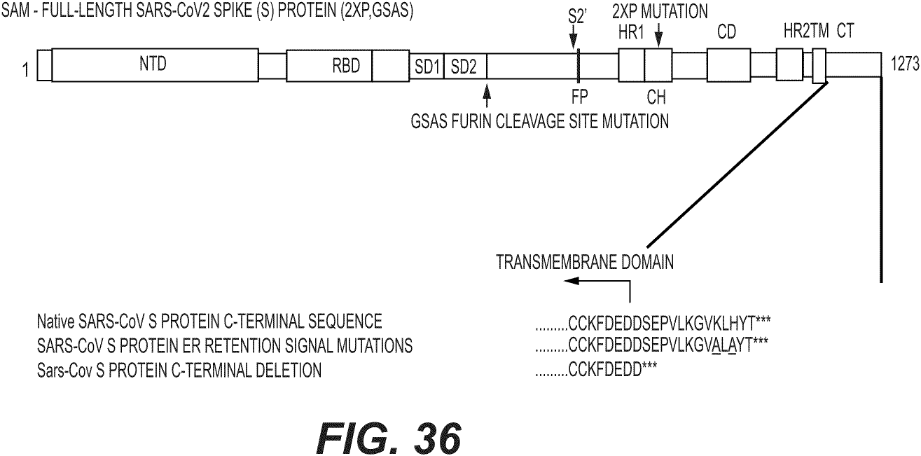

The present inventors introduced further modifications with the aim of increasing the expression and secretion of the ecto domain version of the SARS-CoV Spike (S) protein or the cell-surface expression of the full-length version of the protein. In most constructs, the 16 or 18 N-terminal residues (putative SARS-CoV S signal sequence) of SARS-CoV S protein were replaced by heterologous signal sequences selected from among those that are known to promote good expression and secretion from plasmids. See, e.g., Cat. No. OGS1498, PSF-CMV-PURO-NH2-GAUS - Gaussia (Luciferase) Secretion Plasmid, Sigma-Adrich. Those selected by the inventors for use with self-replicating RNA systems are set forth in Table 1, FIG. 34, FIG. 35. In addition, in order to prevent retention of the protein in the endoplasmic reticulum (ER), mutations were introduced in the ER retention signal (K1269A and H1271A) or the entire C-terminal domain was deleted (residues 1261-1273) in certain full length constructs, as described by McBride, C. E., et al. (2007). “The cytoplasmic tail of the severe acute respiratory syndrome coronavirus spike protein contains a novel endoplasmic reticulum retrieval signal that binds COPI and promotes interaction with membrane protein.” J Virol 81(5): 2418-2428.

All designed SARS-COV2 S protein ectodomain and full length constructs are shown in Tables 2 and 3 respectively.

TABLE 1

Heterologous signal sequences

| | | | |
|---|---|---|---|
| Signal sequence | Protein sequence | DNA sequence | RNA sequence |
| Gaussian Luciferase signal sequence | SEQ ID NO:2 | SEQ ID NO:13 | SEQ ID NO:258 |
| human CD5 signal sequence | SEQ ID NO:3 | SEQ ID NO:14 | SEQ ID NO:259 |
| human CD33 signal sequence | SEQ ID NO:4 | SEQ ID NO:15 | SEQ ID NO:260 |
| human IL2 signal sequence | SEQ ID NO:5 | SEQ ID NO:16 | SEQ ID NO:261 |
| human IgE signal sequence | SEQ ID NO:6 | SEQ ID NO:17 | SEQ ID NO:262 |
| human Light Chain Kappa signal sequence | SEQ ID NO:7 | SEQ ID NO:18 | SEQ ID NO:263 |
| JEV short signal sequence | SEQ ID NO:8 | SEQ ID NO:19 | SEQ ID NO:264 |
| JEV long signal sequence | SEQ ID NO:9 | SEQ ID NO:20 | SEQ ID NO:265 |
| Mouse Light Chain Kappa signal sequence | SEQ ID NO:10 | SEQ ID NO:21 | SEQ ID NO:266 |
| SSP signal sequence | SEQ ID NO:11 | SEQ ID NO:22 | SEQ ID NO:267 |
| Gaussian Luciferase (AKP) signal sequence | SEQ ID NO:12 | SEQ ID NO:23 | SEQ ID NO:268 |

All ecto domain constructs contain proline substitutions K986P and V987P, a “GSAS” (SEQ ID NO:190) substitution (residues 682-685) in the furin cleavage site, and the replacement of the entire C-terminus (after amino acid 1208) by a T4 fibritin trimerization motif (foldon)

TABLE 2

Ecto domain SARS-COV2 S protein constructs

| Construct | Signal sequence | SEQ ID NOs Protein DNA RNA | | |
|---|---|---|---|---|
| Construct B - pJW18 (COVID19 SPIKE Protein with 2Xp_GSAS_T4 Trimer Motif); (also referred to as ‘Spike_ECTO-2P SAM’ and ‘SARS-CoV-2 spike Ecto SAM’) | Native | 26 | 74 | 122 |
| pKL049 (COVID19 SPIKE Protein with 2Xp_GSAS_T4 Trimer Motif_GauLUCss(16aa)) | First 16 amino acids have been replaced by Gaussian Luciferase signal sequence | 27 | 75 | 123 |
| pKL050 (COVID19 SPIKE Protein with 2Xp_GSAS_T4 Trimer Motif_GauLUCss(18aa)) | First 18 amino acids replaced by Gaussian Luciferase signal sequence | 28 | 76 | 124 |
| pKL051 (COVID19 SPIKE Protein with 2Xp_GSAS_T4 Trimer Motif_CD5ss(16aa)) | First 16 amino acids have been replaced by human CD5 signal sequence | 29 | 77 | 125 |
| pKL052 (COVID19 SPIKE Protein with 2Xp_GSAS_T4 Trimer Motif_CD5ss(18aa)) | First 18 amino acids replaced by human CD5 signal sequence | 30 | 78 | 126 |
| pKL053 (COVID19 SPIKE Protein with 2Xp_GSAS_T4 Trimer Motif_CD33ss(16aa)) | First 16 amino acids have been replaced by human CD33 signal sequence | 31 | 79 | 127 |
| pKL054 (COVID19 SPIKE Protein with 2Xp_GSAS_T4 Trimer Motif_CD33ss(18aa)) | First 18 amino acids replaced by human CD33 signal sequence | 32 | 80 | 128 |
| pKL055 (COVID19 SPIKE Protein with 2Xp_GSAS_T4 Trimer Motif_hIL2ss(16aa)) | First 16 amino acids have been replaced by human Interleukin-2 signal sequence | 33 | 81 | 129 |
| pKL056 (COVID19 SPIKE Protein with 2Xp_GSAS_T4 Trimer Motif_hIL2ss(18aa)) | First 18 amino acids replaced by human Interleukin-2 signal sequence | 34 | 82 | 130 |
| pKL057 (COVID19 SPIKE Protein with 2Xp_GSAS_T4 Trimer Motif_hlgEss(16aa)) | First 16 amino acids have been replaced by human IgE signal sequence | 35 | 83 | 131 |
| pKL058 (COVID19 SPIKE Protein with 2Xp_GSAS_T4 Trimer Motif_hlgEss(18aa)) | First 18 amino acids replaced by human IgE signal sequence | 36 | 84 | 132 |
| pKL059 (COVID19 SPIKE Protein with 2Xp_GSAS_T4 Trimer Motif_hLCKss(16aa)) | First 16 amino acids have been replaced by human Light Chain Kappa signal sequence | 37 | 85 | 133 |
| pKL060 (COVID19 SPIKE Protein with 2Xp_GSAS_T4 Trimer Motif_hLCKss(18aa)) | First 18 amino acids replaced by human Light Chain Kappa signal sequence | 38 | 86 | 134 |
| pKL061 (COVID19 SPIKE Protein with 2Xp_GSAS_T4 Trimer Motif_JEVSss(16aa)) | First 16 amino acids have been replaced by JEV short signal sequence | 39 | 87 | 135 |
| pKL062 (COVID19 SPIKE Protein with 2Xp_GSAS_T4 Trimer Motif_JEVSss(18aa)) | First 18 amino acids replaced by JEV short signal sequence | 40 | 88 | 136 |
| pKL063 (COVID19 SPIKE Protein with 2Xp_GSAS_T4 Trimer Motif_JEVLss(16aa)) | First 16 amino acids have been replaced by JEV long signal sequence | 41 | 89 | 137 |
| pKL064 (COVID19 SPIKE Protein with 2Xp_GSAS_T4 Trimer Motif_JEVLss(18aa)) | First 18 amino acids replaced by JEV long signal sequence | 42 | 90 | 138 |
| pKL065 (COVID19 SPIKE Protein with 2Xp_GSAS_T4 Trimer Motif_mLCKss(16aa)) | First 16 amino acids have been replaced by mouse Light Chain Kappa signal sequence | 43 | 91 | 139 |
| pKL066 (COVID19 SPIKE Protein with 2Xp_GSAS_T4 Trimer Motif_mLCKss(18aa)) | First 18 amino acids replaced by mouse Light Chain Kappa signal sequence | 44 | 92 | 140 |
| pKL067 (COVID19 SPIKE Protein with 2Xp_GSAS_T4 Trimer Motif_SSPss(16aa)) | First 16 amino acids have been replaced by SSP signal sequence | 45 | 93 | 141 |
| pKL068 (COVID19 SPIKE Protein with 2Xp_GSAS_T4 Trimer Motif_SSPss(18aa)) | First 18 amino acids replaced by SSP signal sequence | 46 | 94 | 142 |
| pKL069 (COVID19 SPIKE Protein with 2Xp_GSAS_T4 Trimer Motif_GauLUC(AKP)ss(16aa)) | First 16 amino acids have been replaced by Gaussian Luciferase (AKP) signal sequence | 47 | 95 | 143 |
| pKL070 (COVID19 SPIKE Protein with 2Xp_GSAS_T4 Trimer Motif_ GauLUC(AKP)ss(18aa)) | First 18 amino acids replaced by Gaussian Luciferase (AKP) signal sequence | 48 | 96 | 144 |

All full length constructs contain proline substitutions K986P and V987P and a “GSAS” (SEQ D NO:190) substitution (residues 682-685) in the furin cleavage site

TABLE 3

Full length SARS-COV2 S protein constructs

| Construct | Signal sequence | ER mutations | SEQ ID NOs Protein DNA RNA | | |
|---|---|---|---|---|---|
| Construct A - pJW019 (COVID19 SPIKE Protein with 2Xp_GSAS_FL_) (also referred to as ‘Spike_FL-2P SAM’ and ‘SARS-CoV-2 spike full length SAM’) | Native | | 49 | 97 | 145 |
| pKL025 (COVID19 SPIKE Protein with 2Xp_GSAS_FL_ER_ret) | Native | mutations K1269A and H1271A | 50 | 98 | 146 |
| pKL026 (COVID19 SPIKE Protein with 2Xp_GSAS_FL_C_term_del) | Native | C-terminal residues 1261-1273 deleted | 51 | 99 | 147 |
| pKL027 (COVID19 SPIKE Protein with 2Xp_GSAS_FL_ GauLUCss(16aa)) | First 16 amino acids have been replaced by Gaussian Luciferase signal sequence | None | 52 | 100 | 148 |
| pKL028 (COVID19 SPIKE Protein with 2Xp_GSAS_FL_ GauLUCss(18aa)) | First 18 amino acids replaced by Gaussian Luciferase signal sequence | None | 53 | 101 | 149 |
| pKL029 (COVID19 SPIKE Protein with 2Xp_GSAS_FL_ CD5ss(16aa)) | First 16 amino acids have been replaced by human CD5 signal sequence | None | 54 | 102 | 150 |
| pKL030 (COVID19 SPIKE Protein with 2Xp_GSAS_FL_ CD5ss(18aa)) | First 18 amino acids replaced by human CD5 signal sequence | None | 55 | 103 | 151 |
| pKL031 (COVID19 SPIKE Protein with 2Xp_GSAS_FL_ CD33ss(16aa)) | First 16 amino acids have been replaced by human CD33 signal sequence | None | 56 | 104 | 152 |
| pKL032 (COVID19 SPIKE Protein with 2Xp_GSAS_FL_ CD33ss(18aa) | First 18 amino acids replaced by human CD33 signal sequence | None | 57 | 105 | 153 |
| pKL033 (COVID19 SPIKE Protein with 2Xp_GSAS_FL_ hIL2ss(16aa)) | First 16 amino acids have been replaced by human Interleukin 2 signal sequence | None | 58 | 106 | 154 |
| pKL034 (COVID19 SPIKE Protein with 2Xp_GSAS_FL_ hIL2ss(18aa)) | First 18 amino acids replaced by human Interleukin 2 signal sequence | None | 59 | 107 | 155 |
| pKL035 (COVID19 SPIKE Protein with 2Xp_GSAS_FL_ hlgEss(16aa)) | First 16 amino acids have been replaced by human IgE signal sequence | None | 60 | 108 | 156 |
| pKL036 (COVID19 SPIKE Protein with 2Xp_GSAS_FL_ hlgEss(18aa)) | First 18 amino acids replaced by human IgE signal sequence | None | 61 | 109 | 157 |
| pKL037 (COVID19 SPIKE Protein with 2Xp_GSAS_FL_ hLCKss(16aa)) | First 16 amino acids have been replaced by human Light Chain Kappa signal sequence | None | 62 | 110 | 158 |
| pKL038 (COVID19 SPIKE Protein with 2Xp_GSAS_FL_ hLCKss(18aa)) | First 18 amino acids replaced by human Light Chain Kappa signal sequence | None | 63 | 111 | 159 |
| pKL039 (COVID19 SPIKE Protein with 2Xp_GSAS_FL_ JEVSss(16aa)) | First 16 amino acids have been replaced by JEV short signal sequence | None | 64 | 112 | 160 |
| pKL040 (COVID19 SPIKE Protein with 2Xp_GSAS_FL_ JEVSss(18aa)) | First 18 amino acids replaced by JEV short signal sequence | None | 65 | 113 | 161 |
| pKL041 (COVID19 SPIKE Protein with 2Xp_GSAS_FL_ JEVLss(16aa)) | First 16 amino acids have been replaced by JEV long signal sequence | None | 66 | 114 | 162 |
| pKL0442 (COVID19 SPIKE Protein with 2Xp_GSAS_FL_ JEVLss(18aa)) | First 18 amino acids replaced by JEV long signal sequence | None | 67 | 115 | 163 |
| pKL043 (COVID19 SPIKE Protein with 2Xp_GSAS_FL_ mLCKss(16aa)) | First 16 amino acids have been replaced by mouse Light Chain Kappa signal sequence | None | 68 | 116 | 164 |
| pKL044 (COVID19 SPIKE Protein with 2Xp_GSAS_FL_ mLCKss(18aa)) | First 18 amino acids replaced by mouse Light Chain Kappa signal sequence | None | 69 | 117 | 165 |
| pKL045 (COVID19 SPIKE Protein with 2Xp_GSAS_FL_ SSPss(16aa)) | First 16 amino acids have been replaced by SSP signal sequence | None | 70 | 118 | 166 |
| pKL046 (COVID19 SPIKE Protein with 2Xp_GSAS_FL_ SSPss(18aa)) | First 18 amino acids replaced by SSP signal sequence | None | 71 | 119 | 167 |
| pKL047 (COVID19 SPIKE Protein with 2Xp_GSAS_FL_ GauLUC(AKP)ss(16aa)) | First 16 amino acids have been replaced by Gaussian Luciferase (AKP) signal sequence | None | 72 | 120 | 168 |
| pKL048 (COVID19 SPIKE Protein with 2Xp_GSAS_FL_ GauLUC(AKP)ss(18aa)) | First 18 amino acids replaced by Gaussian Luciferase (AKP) signal sequence | None | 73 | 121 | 169 |

### Evaluation/Study Design

The constructs are evaluated in mammalian cells following electroporation of SAM RNA into BHK cells using the following methods:
a. SAM RNA expression for each of the SAM constructs is tested by using antibodies against dsRNA and flow cytometry.
b. Antigen expression is determined by flow cytometry, immunoblots and immunofluorescence assays, to investigate protein in cell lysates, on the cell surface and on cell supernatant.
c. Antigen folding is determined by binding assays to monoclonal antibodies and/or antigen hACE2 receptor.

Following identification of the most efficient candidate constructs formulation into LNP/CNE based-delivery systems is carried out and testing for antigenicity and immunogenicity is carried out in vivo.

### Example 3. Making SAM DNA for Construct A (pJW019) and Construct B (pJW018)

The generation of DNA encoding SAM comprising Construct A and DNA encoding SAM comprising Construct B involved cloning SARS CoV-2 spike SAM into pDNA constructs pJW16, pJW17, pJW18, pJW19 and pJW20. See FIG. 1. A DNA encoding a SAM comprising wild-type spike protein was also generated (pJW20).

Inset 2

Sequences related to making Construct A and B

| SEQ ID NO | Description of Sequence | SEQ ID NO | Description of Sequence |
|---|---|---|---|
| 176 | SAM RNA including the construct A (full length modified spike) | 206 | Oligo oMS146 |
| 177 | DNA encoding the SAM RNA including the construct A (full length modified spike) | 207 | Oligo oMS147 |
| 178 | SAM RNA including the construct B (Ecto) | 208 | Oligo oMS148 |
| 179 | DNA encoding the SAM RNA including the construct B (Ecto) | 209 | Oligo oMS149 |
| 180 | QHD43416.1 | 210 | Oligo oMS150 |
| 181 | Oligo oJW57 | 211 | Oligo oMS151 |
| 182 | Oligo oJW58 | 212 | Oligo oMS152 |
| 183 | Oligo oJW55 | 213 | Oligo oMS153 |
| 184 | Oligo oJW56 | 214 | Oligo oMS154 |
| 185 | Oligo oJW49 | 215 | Oligo oMS155 |
| 186 | Oligo oJW50 | 216 | Oligo oMS156 |
| 187 | Oligo oJW51 | 217 | Oligo oMS157 |
| 188 | RRAR Amino Acid | 218 | Oligo oMS158 |
| 189 | RRAR Nucleic Acid | 219 | Oligo oMS159 |
| 190 | GSAS Amino Acid | 220 | Oligo oMS160 |
| 191 | GSAS Nucleic Acid | 221 | Oligo oMS161 |
| 192 | Oligo oJW52 | 222 | pJW19_Spike_FL-2P SAM Antigen Nucleotide (Start codon to stop codon): |
| 193 | oJW52 portion Amino Acid | 223 | pJW19_Spike_FL-2P SAM Antigen Protein (Start codon to stop codon) |
| 194 | oJW52 portion Nucleic Acid | 224 | pJW19_Spike_FL-2P SAM SAM (5′UTR-through-PolyA Tail Nucleotide) |
| 195 | oJW52 mutation Amino Acid | 225 | Oligo oJL471 |
| 196 | oJW52 mutation Nucleic Acid | 226 | Oligo oJL472 |
| 197 | Oligo oJW59 | 227 | Oligo oJL473 |
| 198 | Oligo oJW53 | 228 | Oligo oJL474 |
| 199 | Oligo oJW41 | 229 | Oligo oJL475 |
| 200 | Oligo oJW54 | 230 | Oligo oJL23 |
| 201 | Oligo oJL112 | | |
| 202 | Oligo oMS142 | | |
| 203 | Oligo oMS143 | | |
| 204 | Oligo oMS144 | | |
| 205 | Oligo oMS145 | | |

Q5 polymerase site-directed mutagenesis PCR was used to remove two BspQ1 restriction sites from the open reading frame of SARS CoV-2 spike sequence present in pJL-0267 (a pCMV expression plasmid). The resulting pDNA was pJW16.

In this regard, Q5 PCR was performed on pJL-0267 using the oligos oJW57 (SEQ ID NO:180) and oJW58 (SEQ ID NO:181) according to the manufacturer protocols. The Q5 PCR product was treated with KLD enzyme mix as per the manufacturers protocol and samples were transformed into NEB 10-Beta chemically competent cells as per the manufacturers protocol. Bacterial transformations were grown on 2X YT agarose media plates containing 100 ug/ml carbenicillin at 37° C. overnight for 18 hrs. Putative positive clone colonies were inoculated in 2X YT liquid media containing 100 ug/ml carbenicillin at 37° C. overnight for 18 hrs. and were miniprepped the next day. Q5 PCR was performed on several of the putative clones using the oligos oJW55 (SEQ ID NO:183) and 56 (SEQ ID NO:184) according to the manufacturer protocol. The Q5 PCR product was treated with KLD enzyme mix as per the manufacturers protocol and samples were transformed into NEB 10-Beta chemically competent cells as per the manufacturer protocol. Bacterial transformations were grown on 2X YT agarose media plates containing 100 ug/ml carbenicillin at 37° C. overnight for 18 hrs. Putative positive clone colonies were inoculated in 2X YT liquid media containing 100 ug/ml carbenicillin at 37° C. overnight for 18 hrs. and were miniprepped the next day.

Putative clones with the two BspQ1 restriction sites removed from the spike open reading frame in the pCMV expression plasmid (pJW16) were then amplified by PCR producing a linear product to be used in a HIFI DNA assembly reaction to insert gBlocks (double-stranded gene fragments) containing sequence to mutate the furin cleavage site between S1 and S2 (furin cleavage products of substrate S0 molecule), and to insert two prolines into S2 near the fusion peptide, thereby creating a spike sequence that would produce a prefusion stabilized version of the protein. These reactions would create the pJW17 plasmid.

In this regard, Q5 PCR was performed on the pJW16-1 (antigen sequence verified using oJL23 (SEQ ID NO: 230), oJL471-475 (SEQ ID NOs: 225-229)) clone using the oligos oJW49 (SEQ ID NO:185) and oJW50 (SEQ ID NO:186) according to the manufacturer protocols. The linear PCR produce was treated with DPN1 as per the manufacturers protocol and the reaction was purified with an Gel extraction/ PCR clean up kit combo as per the manufacturer’s protocol. The purified linear product was run in a HIFI DNA assembly reaction with gBlocks oJW51 ((SEQ ID NO:187) and Furin RRAR (SEQ ID NO:188) sequence (AGGCGAGCCAGG) (SEQ ID NO: 189) in oJW51 was modified to GSAS (SEQ ID NO: 190) (GGCTCCGCCTCC) (SEQ ID NO: 191). 2X Proline mutation was created in oJW52 by altering RLDKVEAE (SEQ ID NO: 193) (CGATTGGATAAGGTCGAAGCCGAG) (SEQ ID NO: 194) to RLDPPEAE (SEQ ID NO: 195) (CGATTGGATCCCCCCGAAGCCGAG) (SEQ ID NO: 196). HIFI samples were transformed into NEB 10-Beta chemically competent cells as per the manufactures protocol. Bacterial transformations were grown on 2X YT agarose media plates containing 100 ug/ml carbenicillin at 37° C. overnight for 18 hrs. Putative positive clone colonies were inoculated in 2X YT liquid media containing 100 ug/ml carbenicillin at 37° C. overnight for 18 hrs. and were miniprepped the next day. Putative clones containing the prefusion stabilizing mutations in the spike protein within the pCMV expression plasmid (pJW17) were sent for sequencing with oJL23, oJL471-475.

The desired SAM pDNA vectors were cloned by performing HIFI DNA assembly reactions between PCR amplicons derived from the full-length native (pJW16) or the full-length prefusion stabilized (pJW17) sequences and the linear SAM pDNA vector. These reactions would create pJW20 and pJW19, respectively. Additionally, a third amplicon from pJW17, lacking the sequence encoding the trans-membrane domain of the spike, was assembled into the linear SAM pDNA with a gBlock encoding for the T4 fibritin trimerization motif in place of the trans-membrane domain, thereby creating the Ecto domain construct (pJW18).

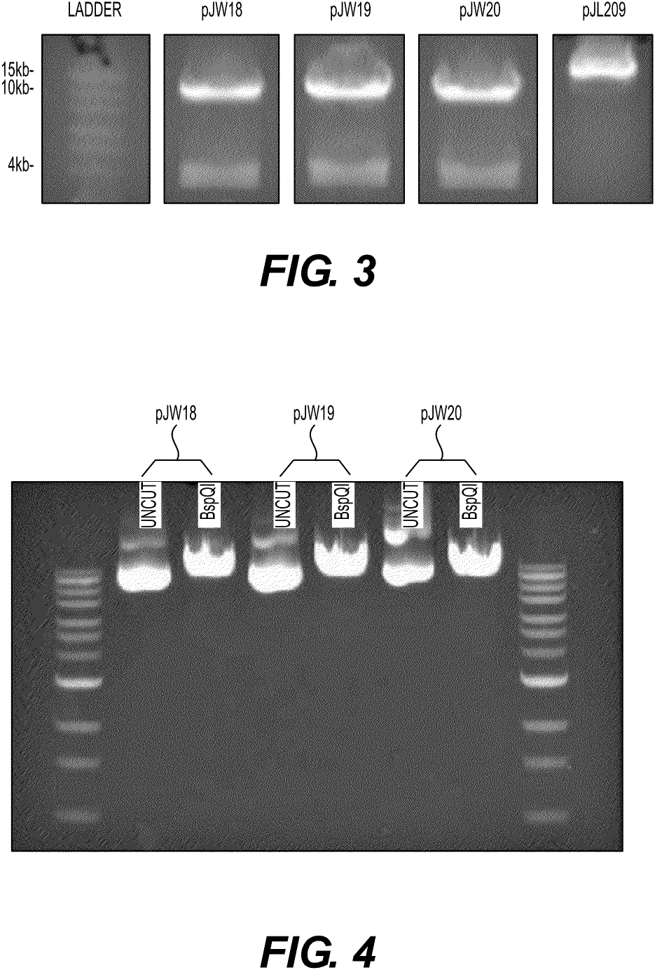

In this regard, the pJL-0209 SAM cloning vector was linearized with Scal restriction enzyme and the linear product was treated with NEB Quick CIP as per the manufacturer’s protocol. PCR amplicons were generated from pJW17 using primer pairs oJW59 (SEQ ID NO: 197)/53(SEQ ID NO: 198) and oJW59/41 (SEQ ID NO: 199). A PCR amplicon was generated from pJW16 using the primer pair oJW59/41. The amplicons were then purified using a Gel Extraction and PCR Purification Combo Kit, and size-verified on a DNA E-gel. The PCR amplicons from pJW17 using primer pairs oJW59/53 was processed in a HIFI DNA assembly reaction with the gBlock oJW54 (SEQ ID NO: 200) and the linear pJL-0209 SAM vector producing putative pJW18 Spike Ecto clones, while the pJW16 and pJW17 oJW59/41 amplicons were processed in a HIFI DNA assembly reaction with the linear vector alone (producing pJW20 Spike Full-length native, and pJW19 Spike Full-length Wrapp mutation stabilized SAM clones, respectively). The T4 Trimerization motif was based on Wrapp et al., 2020 amino acid sequence. The nucleotide sequence from T4 genome (NC_000866.4) was used. The first of the two highlighted Leucine residues below was changed from an F amino acid in the T4 genome sequence (TTC to CTT) to match the published sequence. The trimerization motif is followed directly by a double stop codon. HIFI samples were transformed into NEB 10-Beta chemically competent cells as per the manufactures protocol. Bacterial transformations were grown on 2X YT agarose media plates containing 30 ug/ml kanamycin at 37° C. overnight for 18 hrs. Putative positive clone colonies were inoculated in 2X YT liquid media containing 50 ug/ml kanamycin at 37° C. overnight for 18 hrs and were miniprepped the next day. Putative clones for pJW18, 19, and 20 were verified by a diagnostic digest with Apa1 and Pmel which enables to drop the insert out of the vector backbone, by BspQ1 digestion to confirm only one site and vector linearization, and by sequence analysis of the pDNA maxi preps to verify the antigen, the nsPs, and the untranslated regions of the replicons (FIG. 3). Primers oJL112 ((SEQ ID NO: 201), oJW55, oJW56, and oMS142-161 (SEQ ID NOs: 202-221) were used for sequence analysis.

pDNA in situ maps illustrating the SARS CoV-2 Spike replicon plasmid DNA template sequences, pertinent mutations, and regulatory elements are shown

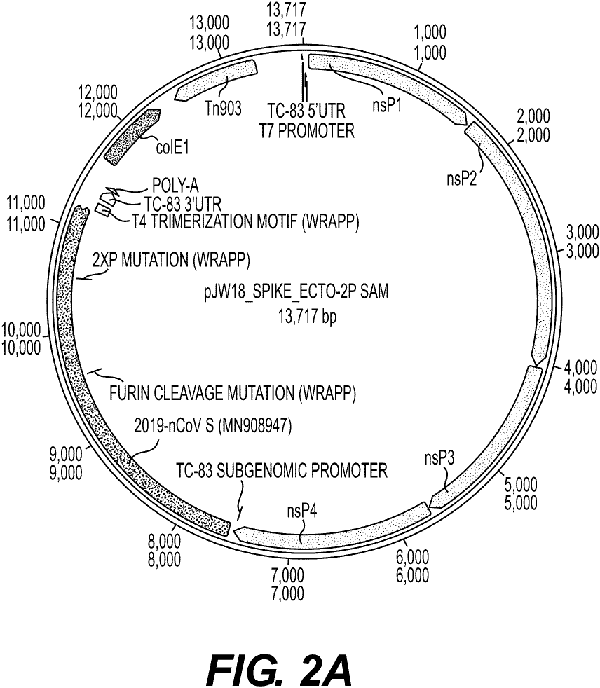

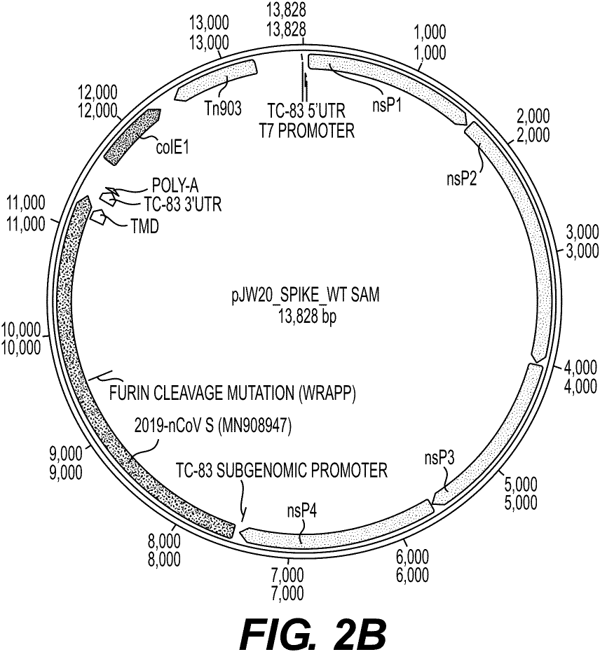

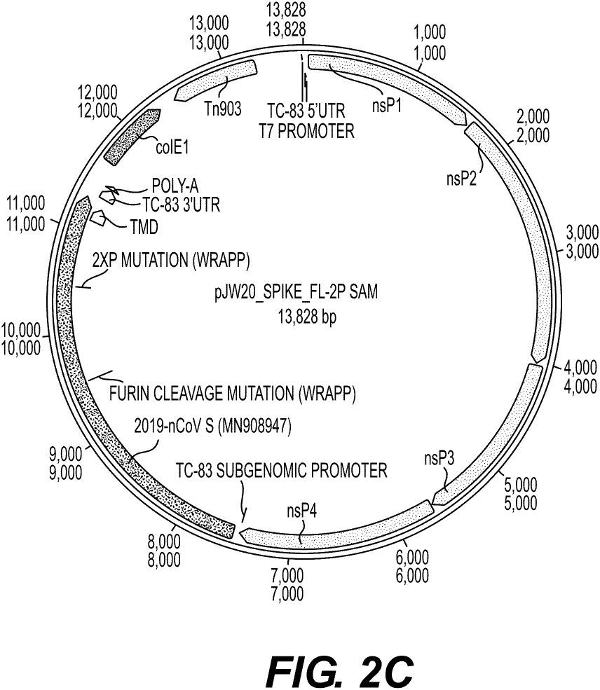

FIG. 2A Spike_ECTO-2P SAM replicon with furin cleavage site mutation and 2X proline mutation, pJW18; FIG. 2B Spike_WT SAM replicon (pJW20). FIG. 2C Spike_FL-2P SAM (pJW19). pJW19_Spike_FL-2P SAM antigen nucleotide (Start codon to stop codon): (SEQ ID NOs: 222); pJW19_ Spike_FL-2P SAM antigen protein (Start codon to stop codon): (SEQ ID NOs: 223); pJW19_ Spike_FL-2P SAM SAM (5′UTR-through-PolyA Tail Nucleotide) (SEQ ID NOs: 223).

### Example 4: In Vitro Synthesis of SAM RNA Having Construct A and SAM RNA Having Construct B

SAM RNA comprising Construct A and SAM RNA comprising Construct B were made from pJW019 and pJW018, respectively. SAM RNA comprising a construct encoding wildtype spike protein was made from pJW020.

Synthetic RNAs were generated by in vitro transcription reactions on linearized plasmid DNA, followed by a Vaccinia capping enzyme reaction to add a 7-methylguanylate cap structure (Cap 0) to the 5′ end of RNAs. Plasmid DNAs (150 µg each) were linearized by incubation with 1x NEB buffer 3.1 and 250 units BspQI restriction enzyme at 50° C. for 2 hours. Linearized DNA templates were purified by mixing with equal volume of phenol/chloroform followed by centrifugation. The aqueous phase was added to clean eppendorf tube and ⅒ volume of 3 M sodium acetate was added to each tube and 2x volume of 100% ethanol. Samples were chilled on ice for 20 minutes, and centrifuged for 30 minutes at 12,000 rpm. Supernatant was removed.

Pellets were washed with 70% ethanol by centrifugation for 5 minutes followed by removal of supernatant. Dried pellets were resuspended in nuclease free water to the final DNA concentration of approximately 0.75 µg/µl. T7 polymerase was used for in vitro synthesis of the RNA in the presence of Tris-HCL, MgCl, DTT, spermidine, pyrophosphatase, RNAse Inhibitor, and ribonucleotides, per protocol. Reactions were incubated for 2 hours at 30° C.

Following the T7 reaction, the vaccinia capping reaction was initiated by adding Tris_HCL, KCL, GTP, SAM, DTT, Turbo DNAse, RNASE Inhibitor and Vaccinia Capping Enzyme according to protocol. 6000 µl of 7.5 M LiCl was added to each reaction and incubated at -20° C. for 30 minutes. RNA was precipitated by centrifugation at 4200 rpm for 30 minutes at 4° C. Supernatant was removed and pellets were washed by adding 5 ml 70% ethanol and centrifugation for 5 minutes 4200 rpm at 4° C.

The pellet was air dried and RNA was dissolved in an approximately 7 ml volume of nuclease-free water. RNA concentration was measured by using a NanoDrop spectrophotometer (Table 4). FIG. 4. illustrates SAM CoV-2 Spike vectors digested with BspQI restriction enzyme prior to IVT. Vector bands were compared to NEB 1 Kb Extend DNA ladder (Ladder).

TABLE 4

Concentration, volume, and total amount of IVT RNA produced for three SARS CoV-2 S replicons

| | Concentration | Total volume | Total RNA |
|---|---|---|---|
| SAM ID | µg/ml | (ml) | (mg) |
| JW18 | 1.71 | 7.00 | 11.90 |
| JW19 | 1.72 | 7.00 | 12.05 |
| JW20 | 1.58 | 7.00 | 11.10 |

### Example 5. Analysis of SAM RNA Having Construct A and SAM RNA Having Construct B by Denaturing Agarose Gel Electrophoresis

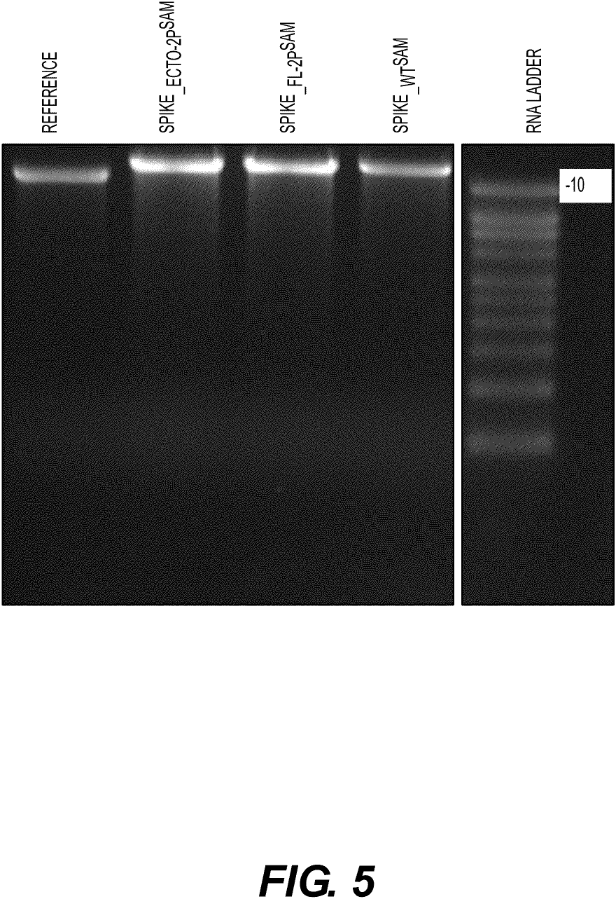

Synthetic RNAs were visualized by agarose gel electrophoresis and quantitated as follows. 600 ng of RNA samples and control RNA Rabies G SAM (RG.co2 SAM Reference STD) and 1000 ng RNA ladder (1000 ng) were added to tubes, with enough RNASE free water to equal 10 ul. 10 µl of 1x NorthernMax®-Gly sample loading dye was added to each tube. Samples were denatured at 50° C. for 20 minutes and electrophoresed on 1% agarose gel in NorthernMax-Gly Gel buffer for 30 minutes at 100 V (5 V per cm distance between anode and cathode). The gel was imaged by BIO_RAD ChemiDoc MP Imaging System (FIG. 5).

### Example 6. In Vitro RNA Expression and Antigen Expression by Western Blot of of SAM RNA Having Construct A and SAM RNA Having Construct B in BHK Cells

### Day 0:

BHK cells were plated 1e⁷/ flask in 4X T225 flasks in Growth Media and incubated at 37° C., 5% CO2 for ~20 hours.

### Day 1:

2 ml DMEM + 1% FBS + P/S (outgrowth media) were added to each well of 9×6 well and kept warm in 37° C. incubator. Electroporator was prepared: 120 V, 25 ms pulse, 0.0 pulse interval, 1 pulse, 2 mm cuvette. Cuvettes were labeled on ice. Cells in growth phase were used, harvested into 5% media (growth) and counted using Countess Cell Counter. 1e⁶ cells per electroporation were used. Each sample were ran in duplicate.

Cells were centrifuged at 1500 rpm (462 × g) for 5 mins and media aspirated. Cells were washed 1× with 20 ml cold Opti-MEM media. Cells were centrifuged at 1500 rpm (462 × g) for 5 mins and media aspirated. The cells were resuspended in Opti-MEM media to 0.25 ml × # of electroporations.

RNA was prepared to a total of 4.2 ug per electroporation. (i.e. 0.1 ug RNA + 4.1 µg Mouse Thymus RNA). For replicates, Master Mix was prepared with both RNAs (for example, for triplicate, prepare 0.3 µg RNA + 12.3 ug MT RNA).

TABLE 5

Reaction

| Construct | Fold RXN | Concentration Construct µg/µl | 100 ng RNA (µl) | Thymus for 100 ng (µl) | 1000 ng RNA (µl) | Thymus for 1000 ng (µl) |
|---|---|---|---|---|---|---|
| A848 | 5x | 1.25 | 0.40 | 50.5 | 4.01 | 16 |
| pJW18-4 | 5x | 1.71 | 0.29 | 20.5 | 2.93 | 16 |
| pJW19-4 | 5x | 1.72 | 0.29 | 20.5 | 2.90 | 16 |
| pJW20-2 | 5x | 1.59 | 0.32 | 20.5 | 3.15 | 16 |
| Thymus Negative | 5x | 1 | xxx | 21 | xxx | 21 |
| | | | Total MT = | 103 | | 85 |

250 µl cells were added into the tube containing the RNA mix and mixed gently 4-5 times. (For replicates, added 250 µl cells/replicate (for example, for 5X, add 1,250 µl cells)). Transferred cells and RNA mixture were added to 2 mm cuvette and proceeded to electroporation. For negative control, 250 µl cells was added to a cuvette (only mouse thymus RNA) and proceeded to electroporation. Electroporated was performed with one pulse. Cells were allowed to rest at room temperature for 10 mins. Cells from cuvettes were added to 6 well dishes...1X EP/ well and incubated at 37° C., 5% CO2 overnight. Added BFA to half of samples (Used Golgi-Plug from BD, 555029) \made 40X BFA in media and aliquoted 50 µl onto cells/ 80 µl stock BFA into 2 mls 1%FBS- DMEM.

### Day 2:

Supernatants:
Collected 2X supernatants (2 ml each) for each construct/ treatment.
Clarifying spin- 1500 RPM, 5 min, 4° C.
Placed supernatant into new tubes.
Placed supernatants at -80° C.

After supernatant removal, cells were placed in PBS and moved to flow processing. ½ of a 6 cm dish of cells was processed to generate lysates. Cells were spun down 1500 RPM, 5 min out of cell dissociation buffer and resuspended in 100 µl cold RIPA +PI. Incubated on ICE 30 min. Spin 10 min full speed to remove nuclei, place lysates in new tube at -80° C.

### FLOW Analysis

Protocol for flow cytometry (6-well) are as follows: Medium was collected and cell monolayer was washed with 2 ml PBS/6-well. PBS was removed and 500 µl cell dissociation buffer enzyme-free/6-well was added and incubate at 37° C. for 10 min. Pipetted multiple times to separate cells and transfer 200 µl cell suspension to an Eppendorf tube. 250 Cell suspension was transferred to 96-well U-bottom plate and spun 1200 rpm for 5 min/ Buffer was discarded. 150 µl Fix/Perm buffer was added, cells were resuspended and incubated at 4° C. for 20 min. Cells were Spun 1200 rpm for 5 min and the buffer discarded. Cells were resuspended with 150 µl Perm buffer. This is the master stock of cells. For each antibody (Ab) staining, 30 µl cells were transferred from the master stock to a new 96-well U-bottom plate. Spin 1200 rpm 5 min, and buffer discarded.

J2 staining: 0.75 µl J2 mAb was mixed with 0.75 µl Zenon APC labeling reagent per sample to be treated and incubate at RT for 5 min. 0.75 µl Zenon blocking reagent mouse IgG was added per sample to be treated and incubated at RT for 5 min. Diluted J2-APC complex with 50 µl Perm buffer per well. 50 µl diluted J2-APC complex was added to the 30 µl cells that were transferred from the master stock to a new 96-well U-bottom plate and incubated at RT for 30 min. The same was spun 1200 rpm for 5 min and buffer discard. The sample was washed with 150 µl Perm buffer, spun 1200 rpm for 5 min and the buffer discard. Cells were resuspended in 150 µl PBS-0.25% BFA and transferred to J2 flow cytometry.

All other Ab staining: Primary Ab was diluted by 1:1000 with Perm buffer. 50 µl diluted primary Ab was added to cells in the 96-well U-bottom plate and incubated at RT for 1 h, and spun 1200 rpm for 5 min and the buffer was discarded. Cells were washed with 150 µl Perm buffer, spun 1200 rpm for 5 min and discard buffer. While washing cells in the previous step, secondary Ab was diluted by 1:1000 with Perm buffer. 50 µl 1:1000 diluted secondary Ab were added to the cells and incubated at RT for 1 h. Spin 1200 rpm for 5 min and discarded buffer. Cells were washed with 150 µl Perm buffer, spun 1200 rpm for 5 min and buffer discarded. Cells were resuspended in 150 µl PBS-0.25% BFA and transfer to flow cytometry.

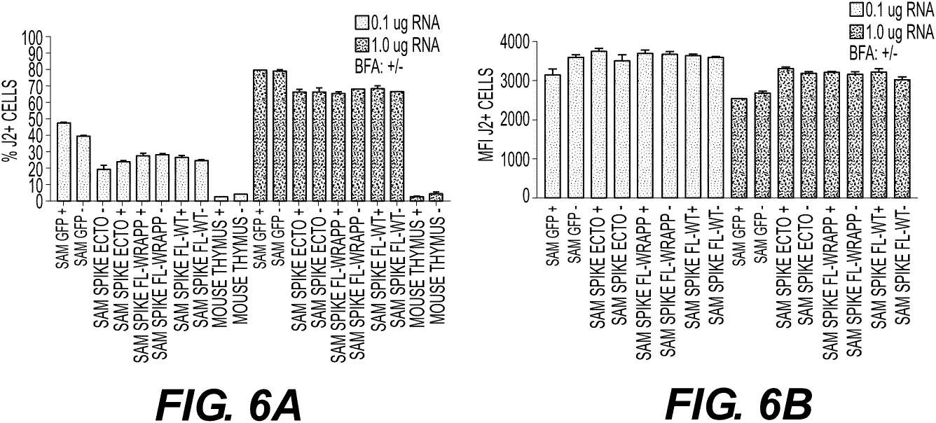

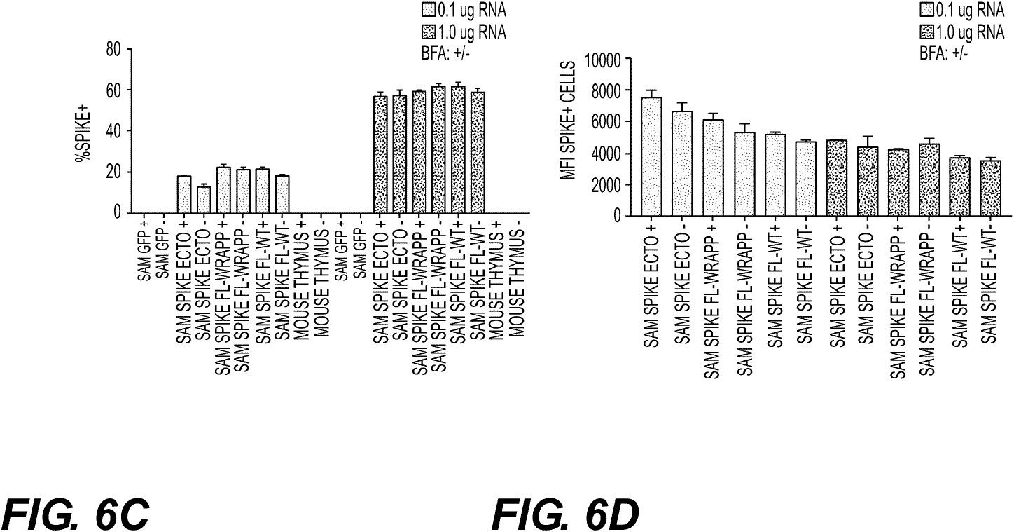

The data suggest that all three spike encoding replicons were potent in BHK cells (FIG. 6A, FIG. 6B, FIG. 6C, FIG. 6D).

### Day 3:

### SDS-PAGE/ Western: Lysates

For analysis, the following was prepared: lysate sample + sample running buffer (10 µl sample (~5% total 6 cm lysate) + 3.5 µl 4X running buffer + 1.5 µl DTT to 50 mM DTT final (4X LDS sample buffer, Invitrogen, NP0007 /// Licor Chameleon DUO ladder, 928-60000).

Samples were incubated at 95° C., 5 min. Standards (8 µl Licor ladder) and samples + sample running buffer were loaded onto 4-12% gel (1.5 mm 10 well, Novex NP0335BOX) and ran for ~50 minutes at 200 V constant in 1X MOPS. Gels were transferred to iBlot 2 Gel Transfer device and transferred to nitrocellulose membrane. Program P0 was ran and adjust stage 3 to 5 min for a total of a ten min transfer. Blocking was performed in 10 ml Oddessy Blocking buffer at RT for 1 hr. while rocking. Primary antibodies were added in Licor Odyssey blocking buffer (927-40000), 0.05% tween and appropriate actin antibody was mixed with effector antibodies and incubated over night at 4° C.
Primary spike antibodies: GTX13560 (SARS-CoV-2 spike antibody, GeneTex), GTX135356 (SARS-CoV-2 spike antibody, GeneTex), GTX632604 (SARS-CoV-2 spike antibody, GeneTex), ProSci4223
Primary Actin antibodies: Mouse anti-Actin Millipore Sigma MAB1501 (Mouse anti-Actin) 1:1000; Rabbit anti-beta-Actin Millipore Sigma MABT523 (Rabbit anti-beta-Actin) 1:1000

### Day 4:

Blots were washed 3X with 1X PBST (0.2% Tween, Ph 7.4), 5 min each and secondary Licor antibodies added for 1 hr. RT.

TABLE 6

Antibodies

| Target | Used at |
|---|---|
| Goat anti-Mouse 680RD | 1:15,000 |
| Goat anti-Rabbit 800CW | 1:15,000 |
| Goat anti-Mouse 800CW | 1:15,000 |
| Goat anti-Rabbit 680RD | 1:15,000 |

Blots were washed 3× 10 min each, 1× 5 min wash PBS only and processed on Licor.

### SDS-PAGE/ Western: Supernatant

10X concentrated supernatant was prepared with Amicon ultra-0.5 10 K (Millipore Sigma, UFC501096) devices, two for each sample. Devices were pre-rinsed with DMEM (No FBS) and 500 µl was place into the device for each sample, spun at 14,000 g for 20 min at 4° C.

Specified supernatant samples were loaded into rinsed columns (500 µl) and spun 20 min. To elute, the columns were flip over into new tube and spun 2 min, 1000x g, 4° C. This resulted in ~100 µl from 1 ml of supernatant once resulting material from two devices were combined.

25 µl of 10X concentrated supernatant were ran on gels with sample buffer and DTT as above. Gels were ran as above and probed supernatant blot with antibody, as above.

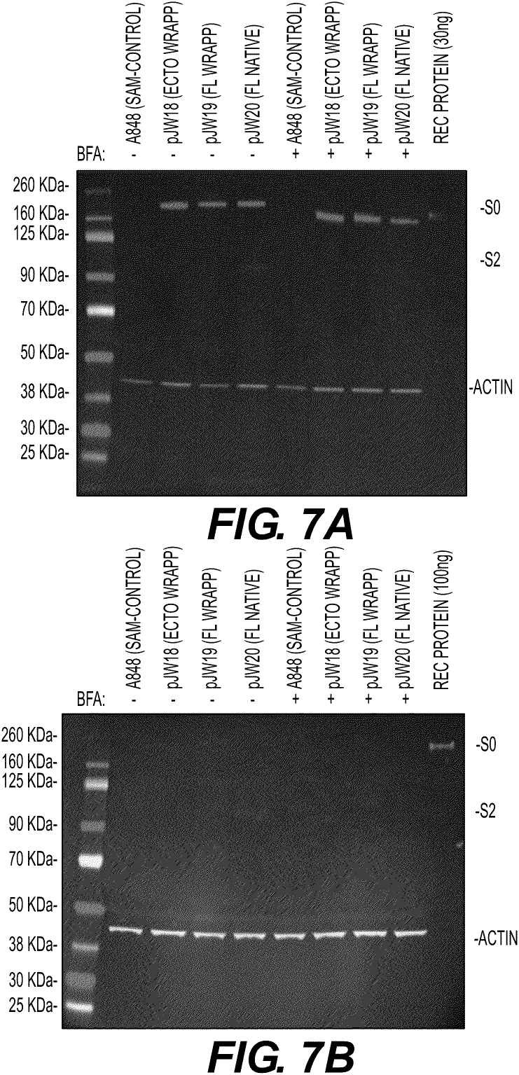

FIG. 7A and FIG. 7B illustrates western blot analysis of SARS CoV-2 Spike SAM RNA replicons from BHK cells. FIG. 7A, 5% of a lysate from a 1 µg RNA electroporation into 1 million BHK cells or FIG. 7B, 25 µl of 10X concentrated supernatant was run per well of an 4-12% SDS-PAGE gel and transferred to a nitrocellulose membrane. The spike protein was probed with the same monoclonal mouse Genetex S2 antibody used for flow cytometry and visualized with a secondary Licor near-infrared antibody. Actin was probed as a loading control. Recombinant S protein (rec protein) was run as an antibody specificity control. S0 is uncleaved spike protein, and KDa= Kilodalton.

### Example 7: In Vitro Surface Expression and hACE2 Binding of Upon SAM Electroporation of BHK Cells With SAM RNA Having Construct A and SAM RNA Having Construct B

BHK cells were electroporated with 1.0 µg IVT SAM SARS CoV-2 replicons RNA. BFA was added to cells four hours after electroporation and seeding. The cells were processed three different ways at 18 hours post-transfection:

### Day 0

BHK cells were plated 1e⁷/ flask in 4X T225 flasks in Growth Media and incubated at 37° C., 5% CO2 for ~20 hrs.

### Day 1

Plates were prepared as follows: 2 ml DMEM + 1% FBS + P/S (outgrowth media) were added to each well of 9×6 well and kept warm in 37° C. incubator. Electroporator was prepared: 120 V, 25 ms pulse, 0.0 pulse interval, 1 pulse, 2 mm cuvette. Cuvettes were labeled and kept on ice.

Cells in growth phase were used. Cells were harvest into 5% media (growth) and count using Countess Cell Counter (Invitrogen). About 1e⁶ cells were used per electroporation. Each sample was run in duplicate. # RNA samples x # replicates= total # of electroporations.

Cells were centrifuged at 1500 rpm (462x g) for 5 mins and the media aspirated. Cells were washed 1X with 20 ml cold Opti-MEM media and centrifuged at 1500 rpm (462x g) for 5 mins. The media was thereafter aspirated. Cells were resuspended in Opti-MEM media to 0.25 ml x # of electroporations. RNA was prepared to a total of 4.2 µg per electroporation. (for example, 0.1 µg RNA + 4.1 µg Mouse Thymus RNA). For replicates, Master Mix was prepared with both RNAs (for example, for triplicate, prepare 0.3 µg RNA + 12.3 µg MT RNA)

TABLE 7

Reaction

| Construct | Fold RXN | Concentration Construct µg/µl | 1000 ng RNA (µl) | Thymus for 1000 ng (µl) |
|---|---|---|---|---|
| mScarlet | 7x | 1.15 | 6.09 | 24.4 |
| pJW18-4 | 7x | 1.71 | 4.10 | 24.4 |
| pJW19-4 | 7x | 1.72 | 4.07 | 24.4 |
| pJW20-2 | 7x | 1.59 | 4.41 | 24.4 |
| Thymus Negative | 7x | 1 | Xxx | 29.4 |
| | | | | 127 |

250 µl cells were added into the tube containing the RNA mix and pipetted gently 4-5 times. For replicates, 250 µl cells/replicate (for example, for 5X, add 1,250 µl cells). Transferred cells and RNA mixture were placed in a 2 mm cuvette and electroporated as above, one pulse. For negative control, 250 µl cells were added to a cuvette (only mouse thymus RNA) and electroporated as above, one pulse.

Cells were allowed to rest at room temperature for 10 mins. Cells from cuvette were added to 6 well dishes...1X EP/ well and incubated at 37° C., 5% CO2 overnight (18 hours). BFA was added to half of samples (Used Golgi-Plug from BD, 555029) \made 40X BFA in media and aliquoted 50 µl onto cells/ 80 µl stock BFA into 2 mls 1%FBS-DMEM.

### Day 3

Samples were collected for flow processing. Processing included Total cell stain (Fixed and Permeabilized), surface stain, and hACE2 binding detection by flow.

FLOW Analysis - Protocol for preparing live cells for flow cytometry from 6-well plate

Plates were generated, medium was removed and washed cell monolayer with 2 ml PBS/well. PBS was removed and 500 µl cell dissociation buffer enzyme-free/well was added and incubated at 37° C. for at least 10 min. Cells were pipetted multiple times to separate cells. 150 µl cells were transferred to 96-well U-bottom plate and kept on ice. Three plates were made for the following assays:
Whole cell staining (Fix/Perm’d; detects both surface and intracellular S protein) with mouse anti-S mAb
Surface staining (live cells) with primary-secondary Ab protocol
hACE2 binding assay (live cells) with primary-secondary Ab protocol

### Whole Cell Staining (Fix/Permeabilized)

Live cells were spun at 1200 rpm for 5 min, and the buffer discarded. 100 µl Fix/Perm buffer was added, and cells resuspended and incubate at 4° C. for 20 min. Cells were spun at 1200 rpm for 5 min and buffer discarded. Cells were resuspend with 150 µl Perm buffer, spun 1200 rpm for 5 min and buffer discarded.

Mouse anti-S mAb - Mouse anti-S mAb was diluted 1:1000 with Perm buffer. 50 µl 1:1000 diluted mouse anti-S mAb were added to the cells, incubated at RT for 1 h and spun 1200 rpm for 5 min, buffer discard. Cells were washed with 150 µl Perm buffer, spun 1200 rpm for 5 min, and buffer discarded.

Goat anti-mouse IgG - Goat anti-mouse IgG Alexa 488 was diluted 1:1000 with Perm buffer. 50 µl 1:1000 diluted goat anti-mouse IgG Alexa 488 were added to cells, incubated at RT for 1 h, spun 1200 rpm for 5 min and buffer discarded. Cells were washed with 150 µl Perm buffer, spun 1200 rpm for 5 min, and buffer discarded. Cells were resuspended cells in 150 µl PBS-0.25% BFA and transfer to flow cytometry.

### Surface Staining (Live Cells) With Primary-Secondary Ab Protocol

Mouse anti-S mAb was diluted 1: 1000 with PBS-2.5% FBS. Live cells were spun at 1200 rpm for 5 min and buffer discarded. 50 µl 1:1000 diluted mouse anti-S mAb was added to the cells and incubate on ice for 30 min, spun 1200 rpm 5 min and buffer discarded. Cells were washed with 150 µl PBS-2.5% FBS, spun 1200 rpm for 5 min, and buffer discarded.

Goat anti-mouse IgG-Goat anti-mouse IgG Alexa 488 was diluted 1:1000 with PBS-2.5% FBS. 50 µl 1:1000 diluted goat anti-mouse IgG Alexa 488 was added to cells, incubate on ice for 30 min, spun 1200 rpm for 5 min and buffer discarded. Cells were washed with 150 µl PBS-2.5% FBS, spun 1200 rpm for 5 min, and buffer discarded. Cells were fixed with 100 µl 1.5% PFA, incubated on ice for 20 min, spun 1200 rpm for 5 min and buffer discarded. Cells were resuspended in 150 µl PBS-0.25% BFA and transferred to flow cytometry.

### hACE2 Binding Assay (Live Cells) With Primary-Secondary Ab Protocol

hACE2 protein (0.31 mg/ml stock) was diluted 1:150 with PBS-2.5% FBS. Live cells were spun at 1200 rpm for 5 min and buffer discarded. 50 µl diluted hACE2 protein was added and incubated on ice for 30 min, spun 1200 rpm 5 min, and buffer discarded. Cells were washed with 150 µl PBS-2.5% FBS, spun 1200 rpm for 5 min and buffer discarded.

Goat anti-hACE2 pAb were diluted 1:200 with PBS-2.5% FBS. 50 µl 1:200 diluted goat anti-hACE2 pAb were added to cells, incubated on ice for 30 min, spun 1200 rpm for 5 min, and buffer discarded. Cell were washed, with 150 µl PBS-2.5% FBS, spun 1200 rpm for 5 min and buffer discarded.

Rabbit anti-goat IgG Alexa 488 were diluted 1:1000 with PBS-2.5% FBS. 50 µl 1:1000 diluted rabbit anti-goat IgG Alexa 488 were added to the cells, incubated on ice for 30 min, spun 1200 rpm for 5 min, and buffer discarded. Cells were washed with 150 µl PBS-2.5% FBS, spun 1200 rpm for 5 min and buffer discarded. Cells were fixed with 100 µl 1.5% PFA, incubated on ice for 20 min. Spin 1200 rpm for 5 min, and buffer discarded. Cells were resuspended in 150 µl PBS-0.25% BSA and transfer to flow cytometry.

### Results: In Vitro Surface Expression and hACE2 Binding of SARS CoV-2 Spike Expressed After SAM RNA Replicons Transfection in BHK Cells by Flow Cytometry

The SAM SARS CoV-2 RNA replicons were transfected into cells with an efficiency (evaluated as percentage of antigen positive cells via anti-dsRNA antibody staining) comparable to the one of a control SAM mScarlet replicon (FIG. 8A). Treatment with BFA resulted in virtually no spike protein or hACE2 on the surface of the cells when BFA was present (FIG. 8B, FIG. 8C). In the absence of BFA the Spike_ECTO-2P SAM expressed protein was not present on the surface of cells. Both Spike_FL-2P SAM (pJW019) and Spike_WT SAM (pJW020) expressed proteins were present on the surface of cells in the absence of BFA treatment. There was slightly more Spike_FL-2P SAM expressed protein on the surface of cells when compared to the Spike_WT SAM expressed protein, and a correlated increase in the hACE2 receptor bound to the surface of cells in the presence of the Spike_FL-2P SAM expressed protein (from pJW019). This phenotype was corroborated by the MFI values of the spike and hACE2 protein on the surface of the cells in the presence of the Spike_FL-2P SAM replicon, which were also slightly higher (FIG. 8D, FIG. 8E, FIG. 8F).

### Example 8: In Vitro RNA Expression, Surface Expression, hACE2 Binding, and Antigen Expression by Western Blot of Muscle Cells Electroporated With SAM RNA Having Construct A and SAM RNA Having Construct B

To corroborate the BHK cell data in a muscle cell line, mouse muscle C2C12 cells were electroporated with 1.0 µg IVT SAM SARS CoV-2 replicon RNAs. BFA was added to cells four hours after electroporation and seeding. The cells were processed in four different ways at 18 hours post-transfection:

### Day 0

muscle cells were plated 1e7/ flask in 6X T225 flasks in Growth Media and incubated at 37° C., 5% CO2 for ~20 hours.

### Day 1

Prepared plates: 2 ml DMEM + 1% FBS + P/S (outgrowth media) were added to each well of 9× 6 well and kept warm in 37° C. incubator. Prepared electroporator: 120 V, 25 ms pulse, 0.0 pulse interval, 1 pulse, 2 mm cuvette. Cuvettes were labeled and kept on ice.

Cells in growth phase were harvested into 5% media (growth) and counted using Countess Cell Counter. ~1e⁶ cells per electroporation were use. Each sample was run in duplicate. # RNA samples x # replicates= total # of electroporations. Cells were centrifuged at 1500 rpm (462x g) for 5 mins and media aspirated.

Cells were washed 1X with 20 ml cold Opti-MEM media and centrifuged at 1500 rpm (462x g) for 5 mins. Media was aspirate. Cells were resuspended in Opti-MEM media to 0.25 ml x # of electroporations. RNA was prepare to a total of 4.2 ug per electroporation. (i.e. 0.1 µg RNA + 4.1 µg Mouse Thymus RNA).

For replicates, Master Mix was prepared with both RNAs (for example for triplicate, prepare 0.3 ug RNA + 12.3 µg MT RNA).

TABLE 8

Reaction

| Construct | Fold RXN | Concentration Construct µg/µl | 1000 ng RNA (µl) | Thymus for 1000 ng (µl) |
|---|---|---|---|---|
| mScarlet (JL-194) | 7x | 1.15 | 6.09 | 24.4 |
| GFP (A848) | 7x | 1.25 | 5.62 | 24.4 |
| pJW18-4 | 7x | 1.71 | 4.10 | 24.4 |
| pJW19-4 | 7x | 1.72 | 4.07 | 24.4 |
| pJW20-2 | 7x | 1.59 | 4.41 | 24.4 |
| Thymus Negative | 7x | 1 | Xxx | 29.4 |
| | | | | 151.4 |

250 µl cells were added into the tube containing the RNA mix and pipetted gently 4-5 times. For replicates, 250 µl cells/replicate was added (for example, for 5X, add 1,250 µl cells). Cells and RNA mixture were transferred to 2 mm cuvette and electroporated as noted above, one pulse. For negative control, 250 µl cells were added to a cuvette add (only mouse thymus RNA) and electroporated as above, one pulse. Cells were allowed to rest at room temperature for 10 mins and added from cuvettes to 6 well dishes...1X EP/ well. Cells were incubated at 37° C., 5% CO2 overnight ~18 hours. hpt: Added BFA to half of samples (Used Golgi-Plug from BD, 555029) \made 40X BFA in media and aliquoted 50 µl onto cells/ 80 µl stock BFA into 2 mls 1%FBS- DMEM.

### Day 2

Processing included Total cell stain (Fixed and Permeabilized), surface stain, and hACE2 binding detection by flow.

### FLOW Analysis - Protocol for Preparing Live Cells for Flow Cytometry From 6-Well Plate

Medium was collected and cell monolayer washed with 2 ml PBS/well. PBS was removed and 500 µl cell dissociation buffer enzyme-free/well was added. The cells were incubated at 37° C. for at least 30 min. Cells were pipetted multiple times to separate cells, 160 µl cell suspension were transferred to an Eppendorf tube and transferred to western blot.

For each assay, 80 µl cells were transferred to 96-well U-bottom plate and keep on ice. 4 plates were made for the following assays:
J2 (dsRNA) staining (Fix/Perm’d)
Whole cell staining (Fix/Perm’d; detects both surface and intracellular S protein) with mouse anti-S mAb
Surface staining (live cells) with primary-secondary Ab protocol
ACE2 binding assay (live cells) with primary-secondary Ab protocol

### J2 (dsRNA) Staining (Fix/Permeabilized)

Live cells were spun at 1200 rpm for 5 min and buffer discarded. 100 µl Fix/Perm buffer was added, cells were resuspend and incubate at 4° C. for 20 min, spun 1200 rpm for 5 min and buffer discarded. Cells were resuspended with 150 µl Perm buffer, spun 1200 rpm for 5 min and buffer discarded. 0.75 µl J2 mAb were mixed with 0.75 µl Zenon APC labeling reagent per each sample to be stained and incubated at RT for 5 min. 0.75 µl Zenon blocking reagent mouse IgG were added per each sample to be stained, and incubated at RT for 5 min. J2-APC complex were diluted with 50 µl Perm buffer per each sample to be stained.

50 µl diluted J2-APC complex were added to cells and incubate at RT for 30 min, spun 1200 rpm for 5 min and buffer discarded. Cells were washed with 150 µl Perm buffer, spun 1200 rpm for 5 min, and buffer discarded. Cells were resuspended in 150 µl PBS-0.25% BSA and transfer to flow cytometry.

### Whole Cell Staining (Fix/Permeabilized)

Live cells were spun at 1200 rpm for 5 min and buffer discarded. 100 µl Fix/Perm buffer was added, cells were resuspended and incubate at 4° C. for 20 min, spun 1200 rpm for 5 min and buffer discarded. Cells were resuspended with 150 µl Perm buffer, spun 1200 rpm for 5 min, and buffer discarded.

Mouse anti-S mAb was diluted 1:1000 with Perm buffer. 50 µl 1:1000 diluted mouse anti-S mAb was added to cells, incubated at RT for 1 h, spun 1200 rpm for 5 min, and buffer discarded. Cells were washed with 150 µl Perm buffer, spun 1200 rpm for 5 min and buffer discarded.

Goat anti-mouse IgG Alexa 488 was diluted 1:1000 with Perm buffer. 50 µl 1:1000 diluted goat anti-mouse IgG Alexa 488 were added to cells, incubated at RT for 1 h, spun 1200 rpm for 5 min and buffer discarded. Cells were washed with 150 µl Perm buffer, spun 1200 rpm for 5 min, and buffer discarded. Cells were resuspended in 150 µl PBS-0.25% BSA and transfer to flow cytometry.

### Surface Staining (Live Cells) With Primary-Secondary Ab Protocol

Mouse anti-S mAb was diluted 1: 1000 with PBS-2.5% FBS. Live cells Spun at 1200 rpm for 5 min, and buffer discarded. 50 µl 1:1000 diluted mouse anti-S mAb were added, incubate on ice for 30 min, spun 1200 rpm 5 min, and buffer discarded. Cells were washed with 150 µl PBS-2.5% FBS, spun 1200 rpm for 5 min, and buffer.

Goat anti-mouse IgG Alexa 488 were diluted by 1:1000 with PBS-2.5% FBS. 50 µl 1:1000 diluted goat anti-mouse IgG Alexa 488 were added to cells, incubated on ice for 30 min, spun 1200 rpm for 5 min, and buffer discarded. Cells were washed with 150 µl PBS-2.5% FBS, spun 1200 rpm for 5 min, and buffer discarded. Cells were fixed with 100 µl 1.5% PFA, incubated on ice for 20 min, spun 1200 rpm for 5 min, and buffer discarded. Cells were resuspended in 150 µl PBS-0.25% BSA and transferred to flow cytometry.

### hACE2 Binding Assay (Live Cells) With Primary-Secondary Ab Protocol

hACE2 protein (0.31 mg/ml stock) was diluted 1:150 with PBS-2.5% FBS. Live cells were spun at 1200 rpm for 5 min, and buffer discarded. 50 µl diluted hACE2 protein were added, incubate on ice for 30 min, spin 1200 rpm 5 min, and buffer discarded. Cells were washed with 150 µl PBS-2.5% FBS, spun 1200 rpm for 5 min, and buffer discarded.

Goat anti-hACE2 pAb were diluted 1:200 with PBS-2.5% FBS. 50 µl 1:200 diluted goat anti-hACE2 pAb were added to cells, incubated on ice for 30 min, spun 1200 rpm for 5 min, and buffer discarded. Cells were washed with 150 µl PBS-2.5% FBS, spun 1200 rpm for 5 min, and buffer discarded.

Donkey anti-goat IgG Alexa 488 were diluted by 1:1000 with PBS-2.5% FBS. 50 µl 1:1000 diluted donkey anti-goat IgG Alexa 488 were added to cells, incubated on ice for 30 min, spun 1200 rpm for 5 min, and buffer discarded. Cells were washed with 150 µl PBS-2.5% FBS, spun 1200 rpm for 5 min, and buffer discarded. Cells were fixed cells with 100 µl 1.5% PFA, incubated on ice for 20 min, spun 1200 rpm for 5 min, and buffer discarded. Cells were resuspended in 150 µl PBS-0.25% BSA and transfer to flow cytometry.

Protein Samples - Supernatants prior to FLOW
Collected 2X supernatants (2 ml each) for each construct/ treatment.
Clarifying spin- 1500 RPM, 5 min, 4° C.
Placed supernatant into new tubes.
Place supernatants at -80° C.

After supernatant removal, cells were placed in PBS and handled for flow processing. ⅓ of a 6 cm dish worth of cells was used to process lysates. Cells were spun down 1500 RPM, 5 min out of cell dissociation buffer and resuspended in 50 µl cold RIPA +PI and incubate on ICE 30 min. Spin 10 min full speed to remove nuclei and place lysates in new tube at -80° C.

### Results: In Vitro RNA Expression, Surface Expression, and hACE2 Binding of SARS CoV-2 Spike SAM RNA Replicons in Muscle Cells by Flow Cytometry.

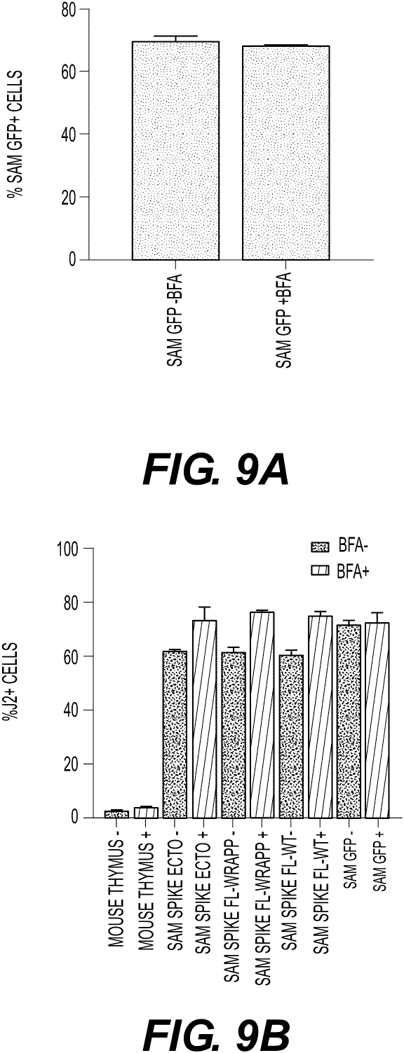

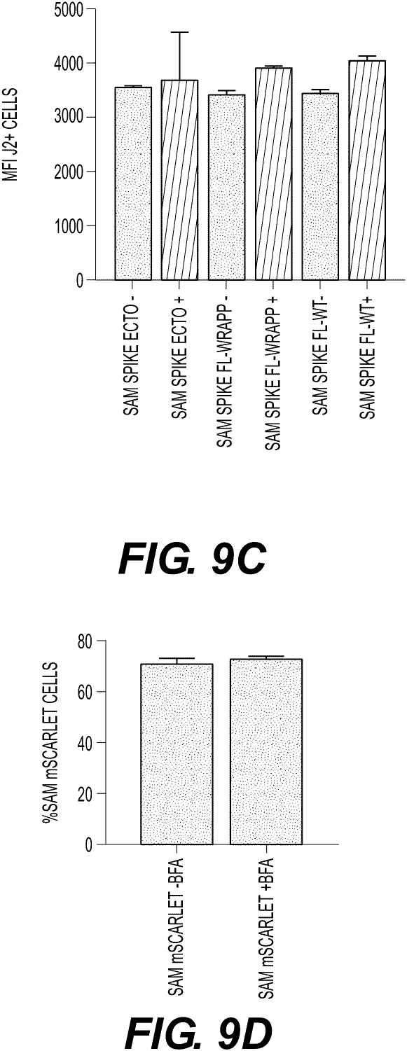

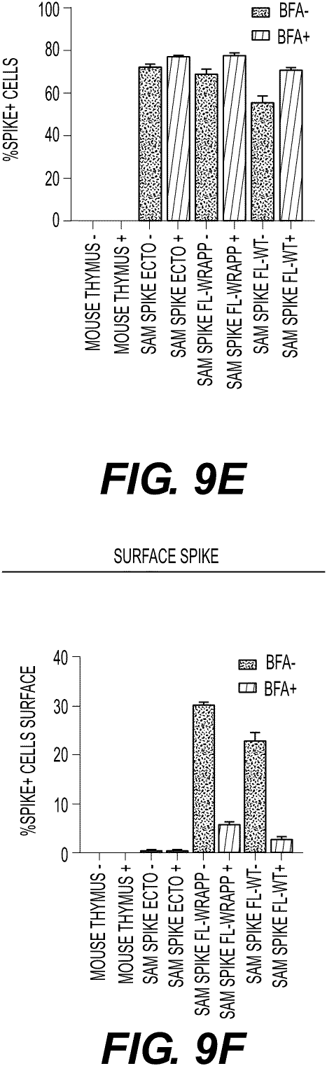

The SAM SARS CoV-2 RNA replicons were transfected into cells comparably to the SAM GFP control replicon, based on the percent positive cell values for J2 dsRNA signal. The MFIs of all three spike replicons were similar, indicating replication to a similar degree for the 3 replicons in muscle cells (FIG. 9A, FIG. 9B, FIG. 9C). The SAM SARS CoV-2 RNA replicons were transfected into cells comparably to the SAM mScarlet control replicon, based on the percent positive dsRNA cell values for total protein in fixed cells (FIG. 9D, FIG. 9E). Similarly to what was observed in BHK cells, treatment with BFA was effective and there was only a small amount of spike protein or hACE2 on the surface of the cells when BFA was present (FIG. 9F, FIG. 9G). In the absence of BFA the Spike_ECTO-2P SAM expressed protein was not present on the surface of cells. Both Spike_FL-2P SAM (pJW019) and Spike_WT SAM (pJW020) derived proteins were present on the surface of cells in the absence of BFA treatment. There was a small amount of Spike_ECTO-2P protein detected on the surface of cells which we determined to be background binding of antibody against hACE2 to cells. There was slightly more Spike_FL-2P SAM expressed protein (pJW019) on the surface of cells when compared to the Spike_WT SAM (pJW020), and a correlated increase in the hACE2 receptor bound to the surface of cells in the presence of the Spike_FL-2P SAM expressed protein.

### Day 4

### Lysates & Supernatant Manipulations - PNGASE Treatment of Lysates:

2 µl Glycoprotein Denaturing Buffer (10X) and 18 µl of LYSATE were used to make 20 µl final volume reaction. They were heated 100 C 10 minutes, in PCR tubes. The sample were chilled on ice, and centrifuge.

5X MM of Glycobuffer, NP-40, and water (1X= 4 µl Glyco2 bf, 4 µl 10% NP40, 12 µl H20) and aliquoted 20 µl into each denatured protein tube (80 µl final). 2 µl PNGase was added to each reaction at 37 C, 1 hr. and -20° C. until day 5.

### Concentration of Supernatants:

10X concentrated supernatant were prepared with Amicon ultra-0.5 10 K (Millipore Sigma, UFC501096) devices, two for each sample. Devices were Pre-rinsed with DMEM (No FBS) and 500 µl were place into device for each sample, spun 14,000 g, 20 min, 4° C. Specified supernatant samples were loaded into rinsed columns (500 µl) and spun 20 min. To elute, sample was flipped over into new tube and spun 2 min, 1000x g, 4° C. This resulted in ~100 µl from 1 ml of supernatant once resulting material from two devices are combined. They were kept at -20° C. until day 5.

### Day 5: Run SDS-PAGE and Western Blot

Prepared lysate sample + sample running buffer (10 µl sample (~5% total 6 cm lysate) + 3.5 µl 4X running buffer + 1.5 µl DTT to 50 mM DTT final (4X LDS sample buffer, Invitrogen, NP0007 /// Licor Chameleon DUO ladder, 928-60000).

Prepared 20 µl of total reaction for (Volume when 4X SB and DTT are added was 30 ul) each PNGASE F treated sample, equivalent to untreated samples.

Ran 25 µl of 10X concentrated supernatant on gels with sample buffer and DTT as above. All samples were incubated at 95° C., 5 min. Loaded standards (8 µl Licor ladder), and samples + sample running buffer onto 4-12% gel (1.5 mm 10 well, Novex NP0335BOX).

TABLE 9

Reaction

| | | | | | | | | | | | | |
|---|---|---|---|---|---|---|---|---|---|---|---|---|
| | Gel# | Lane #: | 1 | 2 | 3 | 4 | 5 | 6 | 7 | 8 | 9 | 10 |
| BFA+/-Lysates | 1 | | Ladder | A848 | pJW18 | pJW19 | pJW20 | A848 | pJW18 | pJW19 | pJW20 | Rec Protein |
| 10 uL lysate loaded | Condition | | | BFA- | BFA- | BFA- | BFA- | BFA+ | BFA+ | BFA+ | BFA+ | 100 ng |
| PNGase+/--BFA Lysates | 2 | | Ladder | A848 | A848 | pJW18 | pJW18 | pJW19 | pJW19 | pJW20 | pJW20 | Rec Protein |
| 10 uL PNGase- | Condition | | | PNGase- | PNGase+ | PNGase- | PNGase+ | PNGase- | PNGase+ | PNGase- | PNGase+ | 100 ng |
| 20 uL PNGase + loaded | | | | | | | | | | | | |
| BFA+/ - 10X Concentrated Supes | 3 | | Ladder | A848 | pJW18 | pJW19 | pJW20 | A848 | pJW18 | pJW19 | pJW20 | Rec Protein |
| 25 uL supe loaded | Condition | | | BFA- | BFA- | BFA- | BFA- | BFA+ | BFA+ | BFA+ | BFA+ | 100 ng |

RAN for ~50 minutes at 200 V constant in 1X MOPS. Gels were transferred to iBlot 2 Gel Transfer device, transfer to nitrocellulose membrane. Ran Program P0, adjusted stage 3 to 5 min for a total of a ten min transfer. Blocked in 10 ml Pierce Protein-Free (PBS) Blocking Buffer, RT 1 hr., rocking. Added primary antibodies in Pierce Protein-Free (PBS) Blocking Buffer, 0.05% tween.

Primary Spike antibodies: GTX632604 1:1000; Primary Actin antibodies: Rabbit anti-beta-Actin Millipore Sigma MABT523 1:5000. Following application of primary antibody, the sample was washed 3X with 1X PBST (0.2% Tween, Ph 7.4), 5 min each. Appropriate secondary antibodies were added 1 hr. at room temperature.

TABLE 10

Antibodies

| Target | Used at |
|---|---|
| Goat anti-Mouse 680RD | 1:15,000 |
| Goat anti-Rabbit 800CW | 1:15,000 |
| Goat anti-Mouse 800CW | 1:15,000 |
| Goat anti-Rabbit 680RD | 1:15,000 |

Thereafter, samples were washed 3X, 10 min each, 1× 5 min wash PBS only and processed on Licor.

### Results:

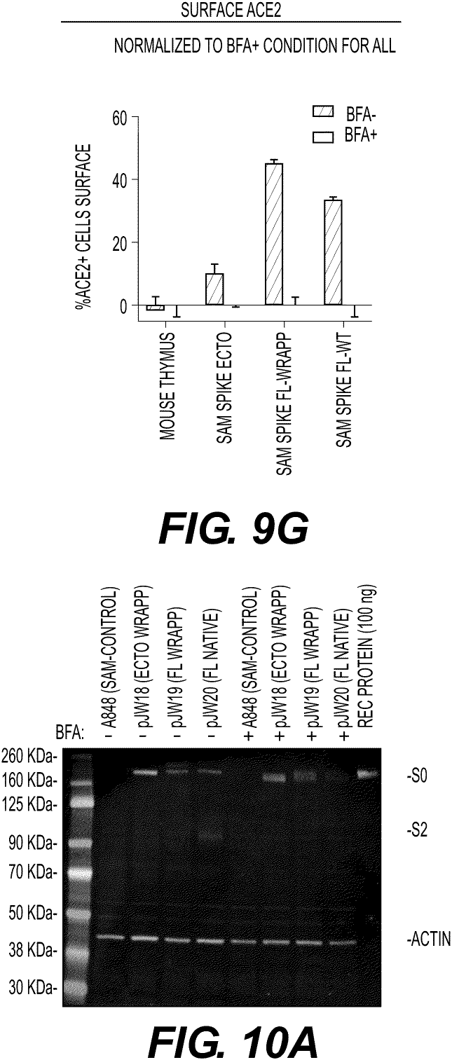

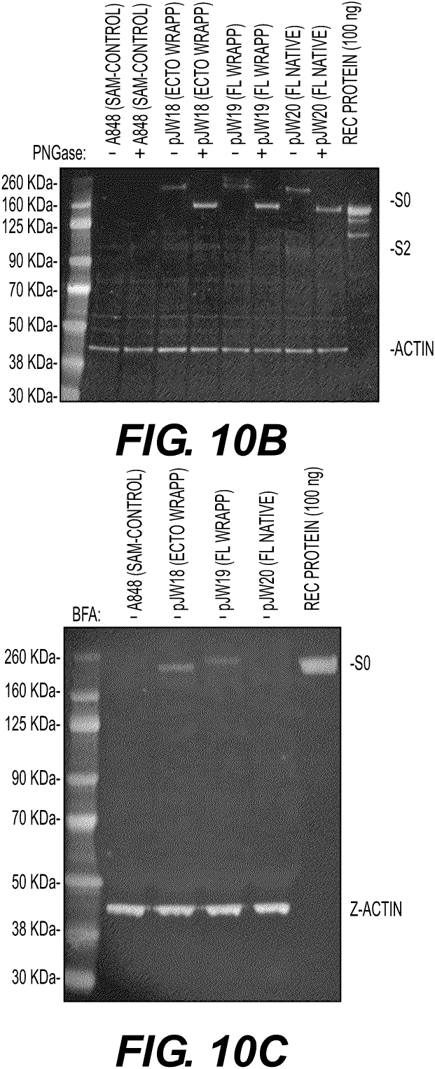

A fraction of the cells analyzed by flow cytometry above were lysed, in the presence and absence of N-glycosidase, and analyzed by western blot (FIG. 10A, FIG. 10B). Additionally, concentrated supernatants from these samples were also analyzed by western blot to evaluate potential secretion of the expressed spike proteins (FIG. 10C). The spike protein was probed with the same monoclonal mouse Genetex S2 antibody used for flow cytometry and visualized with a secondary Licor near-infrared antibody. Actin was probed as a loading control. Recombinant S protein (rec protein) was run as an antibody specificity control. S0 is uncleaved Spike protein, and KDa= Kilodalton. Each of the three SARS CoV-2 replicon RNA expressed spike protein in cell lysates, and very minimal amounts of spike were secreted into the supernatant. Notably, the Spike_ECTO-2P SAM replicon was present to similar amounts within cells in the presence or absence BFA.

### Example 9. In Vitro LNP Protein Expression Potency of SAM RNA Having Construct A and SAM RNA Having Construct B

BHK cells (passage 20) were grown in cell culture media (DMEM with 5% FBS, 1%PSG) at 10,000 cells per well in 96 flat bottom plates using standard BSL-2 cell culture techniques. 4 hours later, applied Tecan liquid handing system process the transfection by adding SAM-LNPs to the cells (3-fold, 8-point dilution transfection in triplication) and incubated overnight. 16 hours later, processed the plates followed by below steps. Fixed the cells with 4% PFA in PBS at volume of 50 µl per well for 15 minutes; permeabilized the cells with 0.05% Trixton-100 at volume of 50 µl per well for 15 minutes for 15 minutes; stained with primary antibody in PBS: SARS-CoV/SARS-CoV-2 S del 10 mouse mAb at 1:1000 dilution at volume of 50 µl per well for 1 hour; stained with secondary antibody Goat anti-Mouse IgG (H+L) Highly Cross-Adsorbed, Alexa Fluor Plus 647 at 1:1000 dilution with Hoechst 33342 at 1:2000 dilution at volume of 50 µl per well for 1 hour. Results were read using a High Content Imaging machine with Compartment Analysis application.

### Results: SAM SARS-CoV2 LNP in Vitro Protein Expression Potency With BHK Cells by High Content Imaging Assay

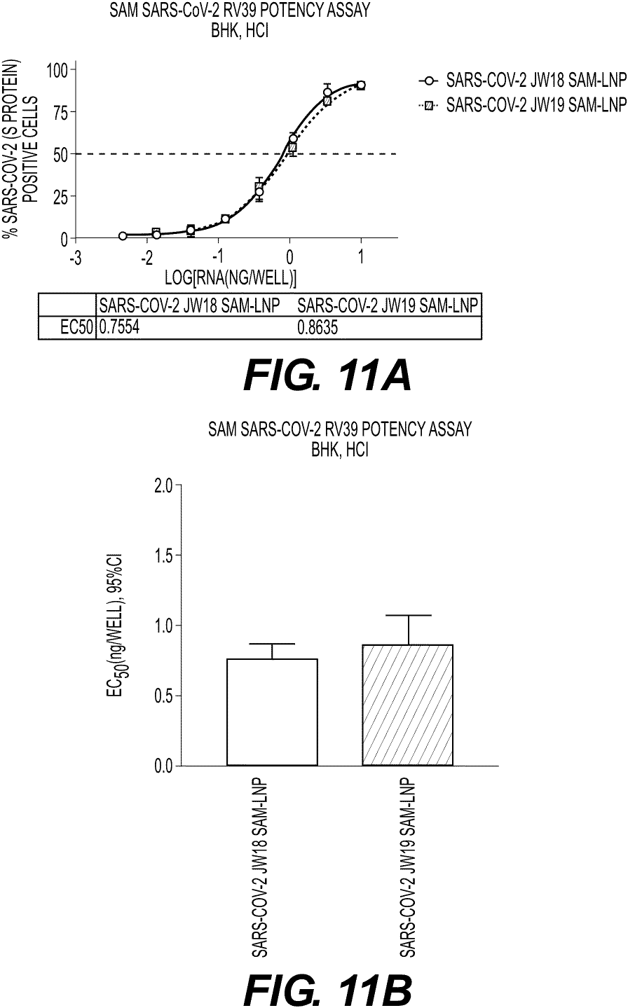

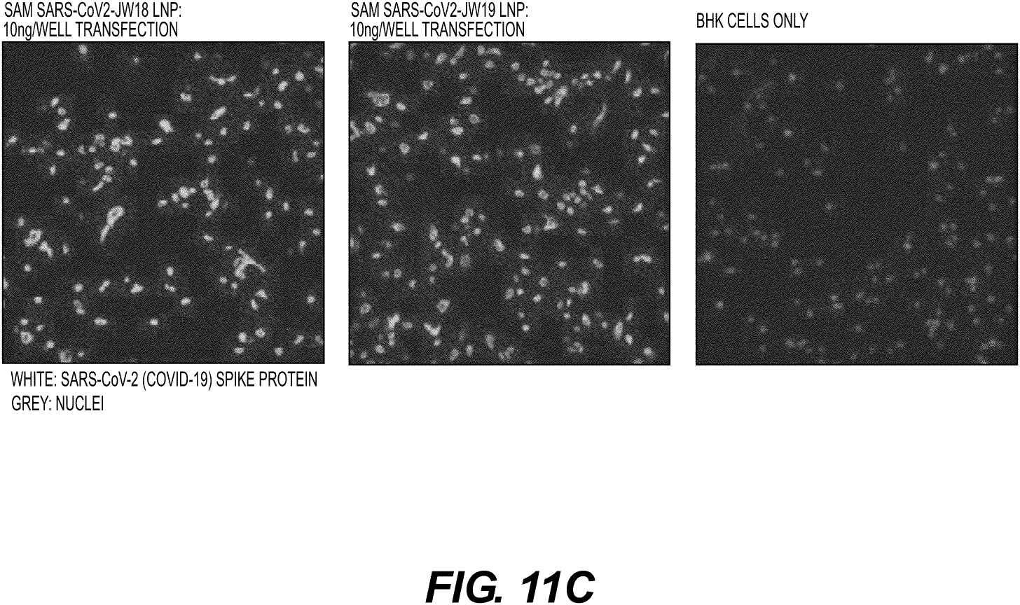

Before transferring the vaccine to immunogenicity evaluation in mice, 2 SAM RNA were formulated with LNP. In vitro LNP protein expression potency assays were performed in BHK cells. Results were measured using a high content imaging machine and are reported as the EC50 value, where lower values indicate a more potent SAM (LNP). The titration curves, bar figure as well as the representative images obtained from the in vitro protein expression potency assays for SARS-CoV-2 SAM (LNPs) were shown in the figures. FIG. 11A depicts EC50 curve, both samples were able to generate EC50 curves successfully. The EC50 of Spike_ECTO-2P SAM (JW18) (LNP) was 0.7554 ng/well, and Spike_FL-2P SAM(JW19) (LNP) was 0.8635 ng/well. FIG. 11B discloses an EC50 bar graph. FIG. 11C depicts results images from HCI 10x objective: JW18: Spike_ECTO-2P SAM (LNP); JW19: Spike_FL-2P SAM (LNP)).

### Example 10: SARSCoV-2 Experimental Vaccine Using SAM RNA Having Construct A and SAM RNA Having Construct B in the Female BALB/c Mouse

Female BALB/c mice (7-8 weeks old) received 2 intramuscular (IM) injections 3 weeks apart in the hind leg thigh muscle, with different doses of (pJW019) SARS-CoV-2 Spike_FL-2P SAM(CNE) (0.15 µg, 1.5 µg or 15 µg), or SARS-CoV-2 Spike_FL-2P SAM (LNP) (0.015 µg, 0.15 µg or 1.5 µg), or (pJW018) SARS-CoV-2 Spike_ECTO-2P SAM (LNP) (0.015 µg and 0.15 µg). Additional groups received a saline solution, or 3 µg of Spike ecto recombinant protein adjuvanted with AS03, following the same schedule of immunization, and used as negative and positive control groups, respectively. Serum samples were collected 21 days after the first immunization (3wp1) and 15 days after the second immunization (2wp2) to assess antibody responses. Spleens and inguinal lymph nodes (LN) were collected 3wp1 from 5 mice in groups 1 and 3 for CMI and B-cell assay development and 2wp2 from 5 mice from all groups to characterize Spike-specific T-cell and germinal center B- cell responses. Details of the study design are provided in FIG. 14.

TABLE 11

Immunization Groups, Geometric Mean Titers, Neutralization Titers for Study Groups

| Group | N | Vaccine | Dose | Formulation | IgG GMT 3wp1 (AU) | IgG GMT 2wp2 (AU) | GMT 50% Neutralization 2wp2 |
|---|---|---|---|---|---|---|---|
| 1 | 10* | Saline | | | 0.004 | 0.004 | 10.00 |
| 2 | 12 | Spike_ECTO-2P protein (Construct B) | 3 µg | AS03 | 27.880 | 1027.000 | 4832.00 |
| 3 | 17 | Spike_FL-2P SAM (Construct A) | 15 µg | CNE | 6.853 | 41.470 | 173.40 |
| 4 | 12 | Spike_FL-2P SAM (Construct A) | 1.5 µg | CNE | 1.499 | 9.828 | 32.62 |
| 5 | 12 | Spike_FL-2P SAM (Construct A) | 0.15 µg | CNE | 0.042 | 1.889 | 11.08 |
| 6 | 12 | Spike_FL-2P SAM (Construct A) | 1.5 µg | LNP | 30.550 | 158.000 | 2483.00 |
| 7 | 12 | Spike_FL-2P SAM (Construct A) | 0.15 µg | LNP | 5.309 | 154.600 | 640.30 |
| 8 | 12 | Spike_FL-2P SAM (Construct A) | 0.015 µg | LNP | 4.489 | 101.500 | 279.90 |
| 9 | 12 | Spike_ECTO-2P SAM (Construct B) | 0.15 µg | LNP | 0.516 | 35.730 | 17.88 |
| 10 | 12 | Spike_ECTO-2P SAM (Construct B) | 0.015 µg | LNP | 0.016 | 1.129 | 10.00 |

* 5 mice from group 1 and 5 mice from group 3 were sacrificed on day 21 for spleen and LN collections for CMI and B-cell assay set up. All mice in the saline group had undetectable titers and were assigned a value of ½ of the Standard Curve LLOQ.

TABLE 12

Dosing

| | |
|---|---|
| Immunization method | Intramuscular- Upper hind leg (thigh) |
| Dose volume | Groups 1, 2 and 6-10: 50 µl/dose equally divided [25µl/hindleg (thigh muscle) for each dose] Groups 3, 4 and 5: 100 µl/dose equally divided [50µl/hindleg (thigh muscle) for each dose] |
| Materials | Insulin syringe with attached 28-gauge needle |
| Frequency | Day 0- 1st dose Day 21- 2nd dose; post 1st serum collection |

### Spike Specific IgG Titers

The titers of Spike-specific serum IgG binding antibodies were measured using a Luminex based assay. Luminex microspheres were covalently coupled with SARS-CoV-2 Spike antigen using sulfo-NHS and EDC according to manufacturer’s instructions. In 96-well plates, 1,500 microspheres/well suspended in 50 µl of PBS, 1% BSA + 0.05% Na Azide (assay buffer) were added to 100 µl of five-fold serially diluted mouse serum. After incubation of the microspheres and serum on an orbital shaker, covered, at RT for 60 min., the microspheres were washed twice with 200 µl/well of PBS, 0.05% Tween-20 (wash buffer) on a plate washer using a magnet to allow settling of beads between washes. After the second wash, the beads were suspended with 50 µl/well of r-Phycoerythrin (r-PE) conjugated anti-mouse IgG, (Fcy Fragment of subclasses 1+2a+2b+3), at a 1:50 dilution. The plates were covered and incubated on an orbital shaker at RT for 60 min. After a final plate wash (same as described above), the samples were resuspended in 80 µl of PBS, covered and incubated at room temperature (RT) on an orbital shaker for 20 minutes. Fluorescence intensity was measured using a Luminex FlexMap 3D. A standard composed of three monoclonal antibodies (mAb) to the S protein added into naïve mouse serum (Table 13) was used to calculate sample titers. The standard was assigned a value of 100 AU (Assay Units). The sample titers were interpolated based on a four parameter logistic fit of the standard curve. Data points for calculation of sample titers were selected from within the range of 10% to 70% of the lower and upper asymptote of the standard curve. The final sample titer was the average of the back calculated titers for dilutions falling within the acceptable range of the standard curve.

TABLE 13

mAb used for the serum standard

| | | |
|---|---|---|
| Source | Catalog# | Specificity to Spike Region |
| Gene Tex | GTX632604 | S2 |
| MP Biomedical | 8720412 | S2 |
| Absolute Antibody | Ab01680-10.0 | S1 |

### Results

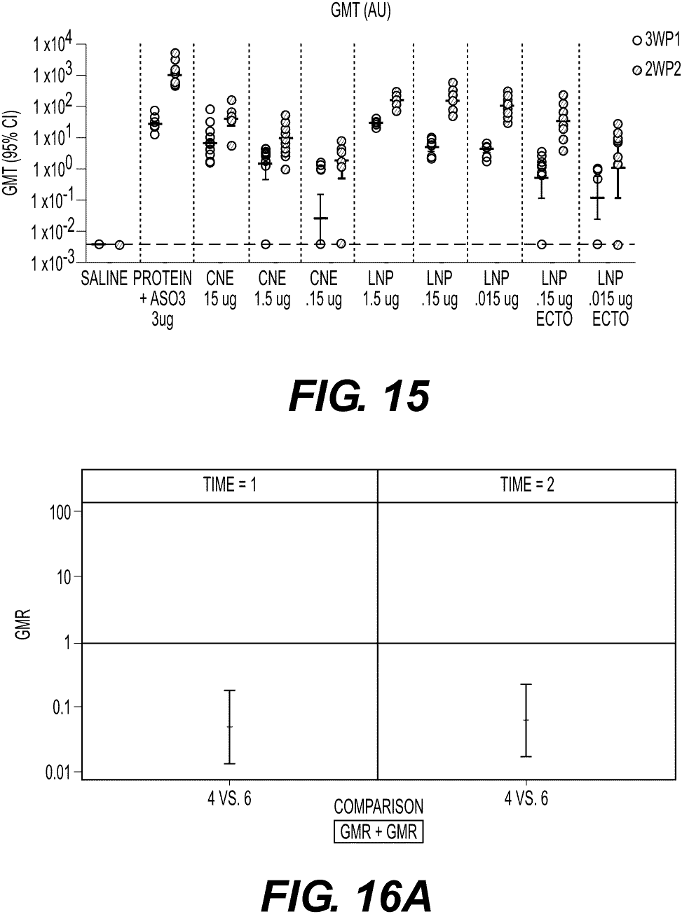

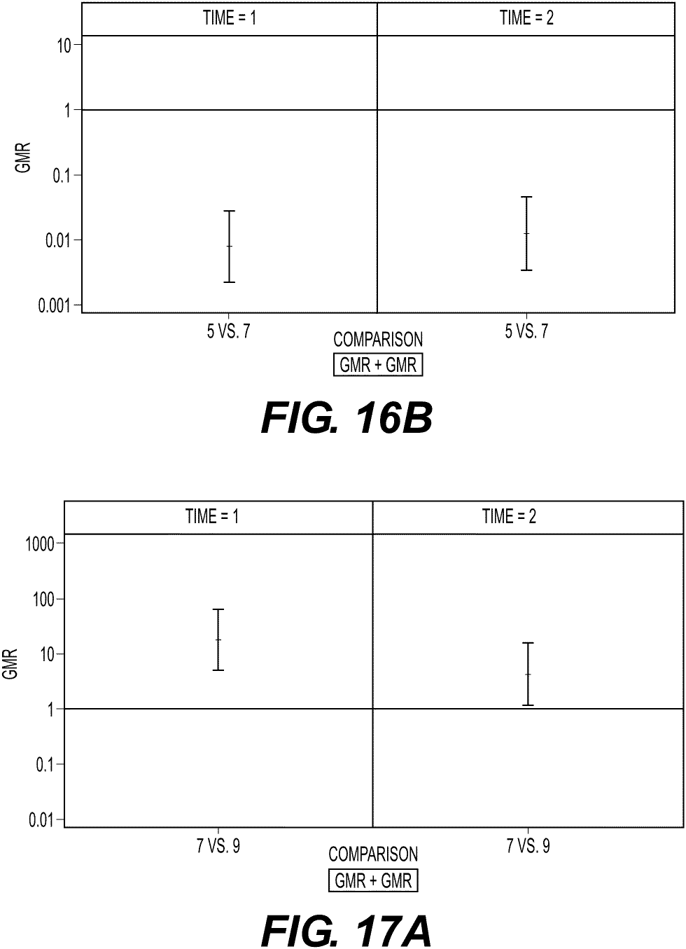

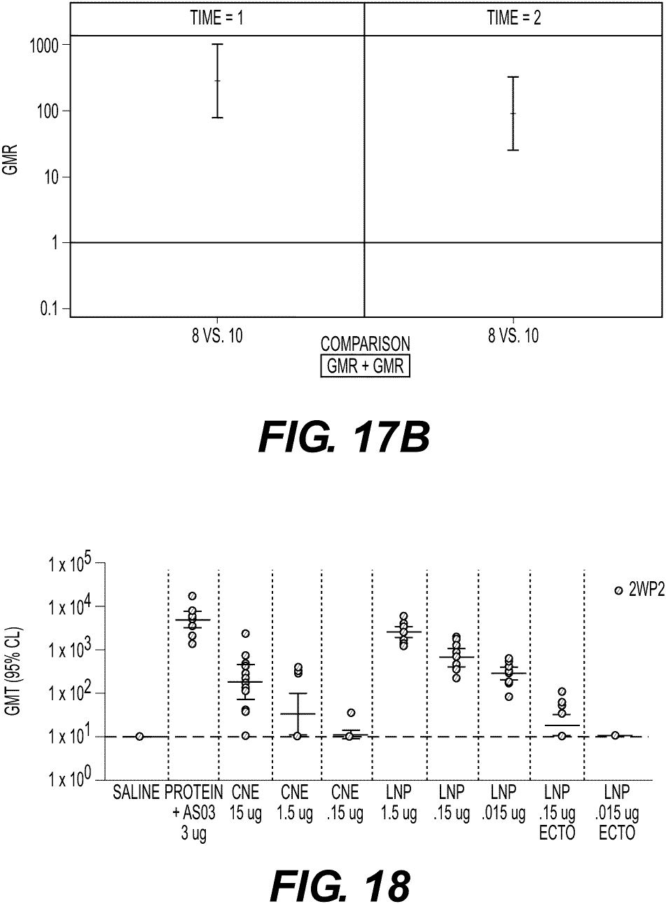

Endpoint titers from each group were combined to calculate Geometric Mean Titers (GMT) with 95% confidence intervals (Table 11 and FIG. 15). Geometric Mean Ratios (GMR) were calculated to demonstrate fold-differences for study groups comparisons (FIG. 16A, FIG. 16B, FIG. 17A and FIG. 17B). Data were analyzed within formulations and between formulations.

The Geometric Mean Titers from all the groups demonstrated that immunization with all SAM formulations expressing Spike protein were immunogenic after one and two doses. Boosting of the immune response was observed after the second dose in all SAM and AS03-adjuvanted Spike protein groups. Comparisons of the lowest dose of Spike_FL-2P SAM (LNP) formulation (0.015 µg) demonstrated that there was a decrease in the IgG GMT as compared to either higher Spike_FL-2P SAM (LNP) formulations (1.5 µg and 0.15 µg) after the second dose. An examination of the GMTs within the CNE and LNP formulation of Spike_FL-2P SAM indicated that there was a dose response for the CNE formulations. This trend was not observed in the LNP formulations where no dose response trend was observed across the three dosing formulations from 1.5 µg to 0.015 µg. Additionally, the GMTs for the 1.5 µg and 0.15 µg dose of LNP had similar titers after the second dose with a GMR of 0.98. The highest IgG binding antibody response was observed with the AS03-adjuvanted Spike_ECTO-2P protein group.

A comparison of the Geometric Mean Ratios for comparable doses of the Spike_FL-2P SAM vaccines demonstrated that the LNP delivery formulations elicited higher IgG binding antibody titers than the CNE delivery formulation at both the 1.5 µg and 0.15 µg dose. The GMR for the 1.5 µg and 0.15 µg dose indicated that the IgG binding antibody response was between 16- to 80-fold higher with the LNP formulation as compared to the CNE formulation. A similar comparison of the Spike_FL-2P SAM to the Spike_ECTO-2P SAM, both formulated in LNP, again demonstrated the higher IgG binding antibody titers for the Full-length Spike SAM formulations. The GMR for the 0.15 µg and 0.015 µg dose indicated that the IgG binding antibody response was between 4-to 90-fold higher with the Spike_FL-2P SAM formulation as compared to the Spike_ECTO-2P SAM formulation.

The Spike-specific IgG binding antibody titers indicated that the SARS-CoV-2 Spike_FL-2P SAM investigational vaccine was immunogenic at all SAM doses tested with either the CNE or LNP delivery formulations. However, the SAM LNP formulations elicited higher IgG binding antibody levels than the corresponding CNE formulations. These data also indicated that there was a dose response for the SARS-CoV-2 Spike_FL-2P SAM (CNE) formulations from the 15 µg to 0.15 µg doses, whereas a dose response was not observed for the LNP formulations, but a trend with a decrease in IgG binding antibody titers was observed when comparing either the 1.5 µg or 0.15 µg dose to the 0.015 µg dose.

### Antibody Neutralization

A neutralization assay using SARS-CoV-2 mNeonGreen reporter virus was used to test the neutralizing activity of serum antibodies. Vero CCL-81 cells (1.2 × 104) in 50 µl of DMEM (Gibco) containing 2% FBS (HyClone) and 100 U/ml Penicillium-Streptomycin (P/S; Gibco) were seeded in each well of black µCLEAR flat-bottom 96-well plate (Greiner Bio-one™). Cells were incubated overnight at 37° C. with 5% CO2. On the following day, serum samples were serially diluted two-fold in 2% FBS and 100 U/ml P/S DMEM, and incubated with mNG SARS-CoV-2 at 37° C. for 1 h. The virus-serum mixture was transferred to the Vero CCL- 81 cell plate with the final multiplicity of infection (MOI) of 0.5. For each serum, the starting dilution was 1/20 with nine two-fold dilutions to the final dilution of 1/5120.

After incubating the infected cells at 37° C. for 16 hrs., 25 µl of Hoechst 33342 Solution (400-fold diluted in Hank’s Balanced Salt Solution; Gibco) were added to each well to stain the cell nucleus. The plate was sealed with Breath-Easy sealing membrane (Diversified Biotech), incubated at 37° C. for 20 min, and quantified for mNG fluorescence on CytationTM 7 (BioTek). The raw images (2 × 2 montage) were acquired using 4× objective, processed, and stitched using the default setting. The total cells (indicated by nucleus staining) and mNG-positive cells were quantified for each well. Infection rates were determined by dividing the mNG-positive cell number with the total cell number. Relative infection rates were obtained by dividing the infection rates of serum-treated wells with the infection rate of the non-serum treated controls well. The curves of the relative infection rates versus the log transformed serum dilutions were plotted using Prism 8 (GraphPad). A 4-parameter logistic curve fit was used to determine the dilution fold that neutralized 50% of mNG fluorescence (NT50). The upper and lower asymptotes of the fitted curve were constrained to 100 and 0. Each serum was tested in duplicate and the final result was the geometric mean of the two results.

### Results:

For each serum, a nonlinear regression method was used to determine the dilution fold that neutralized 50% of mNG fluorescence (NT50), reported in FIG. 18 and Table 11. The NT50 demonstrated that immunization with all Spike_FL-2P SAM (LNP) formulations expressing Spike protein elicited functional antibody titers after two doses. 1.5 µg of SARS-CoV-2 Spike_FL-2P SAM (LNP) and the AS03-adjuvanted Spike protein vaccines induced the highest neutralizing titers among all vaccine groups. SARS-CoV-2 Spike_FL-2P SAM (LNP) at the 1.5 µg dose induced higher neutralizing titers compared to the Spike_FL-2P SAM (LNP) at the 0.15 µg and 0.015 µg doses (p<0.0001).

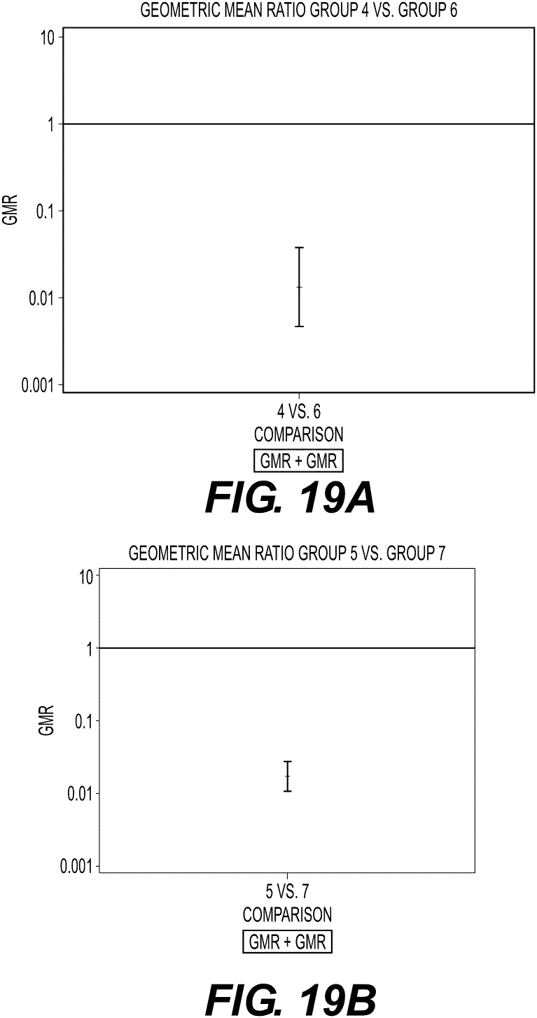

A comparison of the Geometric Mean Ratios for comparable doses of the Spike_FL-2P SAM vaccines demonstrated that the LNP delivery formulations elicited significantly higher neutralizing antibody titers than the CNE delivery formulation at both the 1.5 µg (p<0.0001) and 0.15 µg (p<0.0001) doses. (FIG. 19A, FIG. 19B).

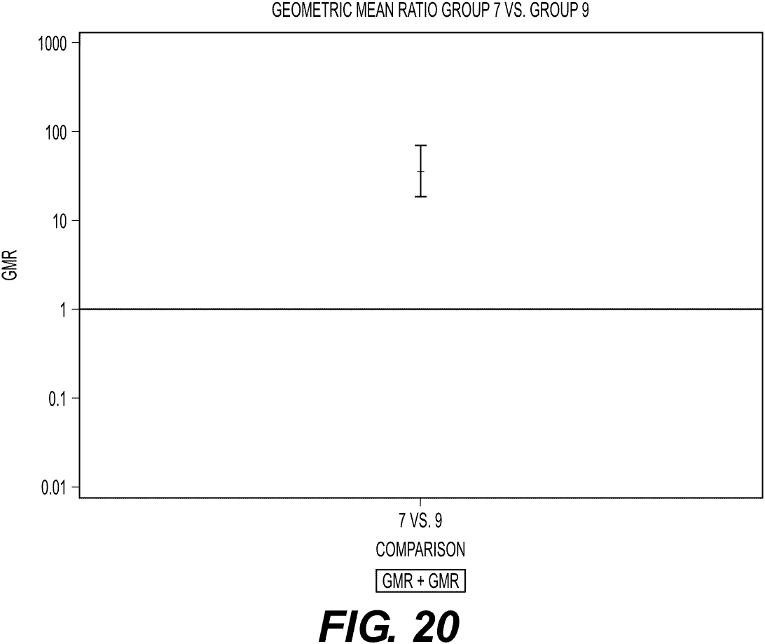

A similar comparison of the Spike_FL-2P SAM to the Spike_ECTO-2P SAM, both with 0.15 µg SAM formulated in LNP, again demonstrated significantly higher neutralizing antibody titers for the Full-length Spike SAM formulations (p<0.0001). (FIG. 20).

The results suggest that the SARS-CoV-2 Spike_FL-2P SAM (LNP) vaccine induced high neutralizing antibody titers against SARS-CoV-2 virus. The Spike_FL-2P SAM (LNP) formulations elicited a dose-dependent response, with higher neutralizing antibody titers than the corresponding Spike_FL-2P SAM (CNE) doses (p<0.0001).

### Spike-Specific B-Cell Responses

Characterization of Spike-specific B-cells from spleen and draining lymph nodes of vaccinated animals was performed using flow cytometry. Cell suspensions prepared from spleen, or pairs of inguinal lymph nodes from each mouse, were stained with near IR live/dead cell stain for 20 min at room temperature and then incubated with Fc block in PBS plus 1% FBS (HyClone, Thermo Scientific) for 10 min at 4° C. Approximately 1-2 x10⁶ splenocytes or LN cells were stained for 30 min at 4° C. with the following mAb: anti-CD3 BUV737, anti-CD19 BV786, anti-IgD BV421, anti-IgM BUV395, anti-GL7 AF647, anti-CD95 BV711, anti-CD138 BB700, anti-CD38 BV650, anti-CD80 PECF594, anti-CD73 PE-Cy7, anti-CD273 PE. To identify Spike-specific B-cells, samples were stained with 1 µg per 1-2 x106 cells of SARS-CoV-2 Spike protein labelled with Alexa Fluor 488.

### Results:

During the generation of vaccine-specific B-cells, upon activation, antigen-specific B cells migrate to the B-cell follicle, in the spleen or the draining lymph nodes, where they lose IgM and IgD membrane expression (class-switching), differentiate into germinal center B-cells, and undergo through the processes of somatic hypermutation and affinity maturation. A characterization of the Spike-specific B-cells induced by the SARS-CoV-2 SAM vaccines was performed in spleens and inguinal draining lymph nodes, 2 weeks after the second immunization (2wp2, Day 36). The frequencies of total and Spike specific class-switched B-cells (identified as CD3-CD19+IgM-IgD- B cells) were measured and their phenotype characterized based on the expression of specific surface markers. Memory B-cells were identified as CD95-CD38+ cells, and germinal center Bcells as GL7+CD95+ cells. Furthermore, CD80, PD-L2 and CD73 expression markers were used to provide further characterization of the Spike-specific B-cells elicited by the SARS-CoV-2 SAM (LNP) vaccine.

### B-Cell Responses in the Spleen at 2wp2

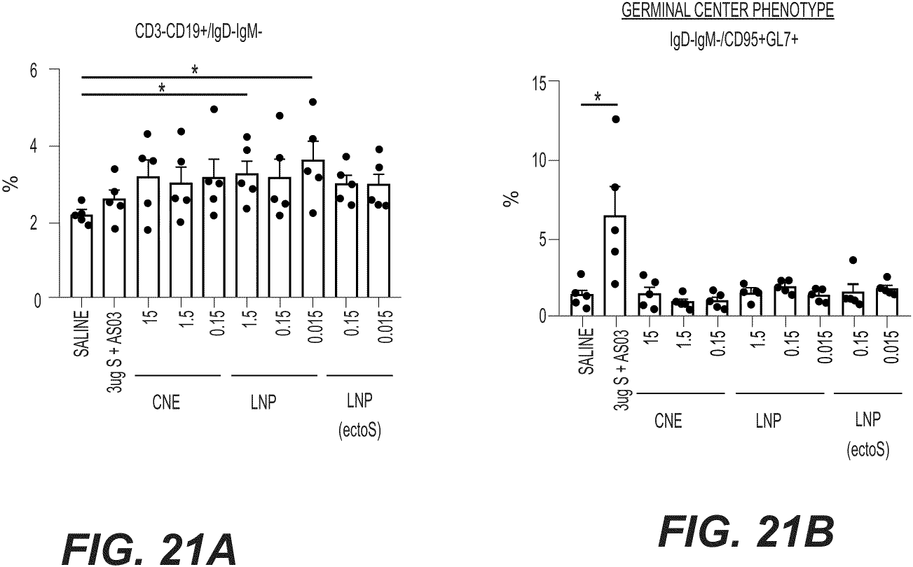

Total B-cell responses in the spleen from all study groups were evaluated 2 weeks after the second vaccination (FIG. 21A, FIG. 21B). Only mice receiving the AS03-adjuvanted recombinant protein vaccine showed germinal center B-cells in the spleen (FIG. 21B).

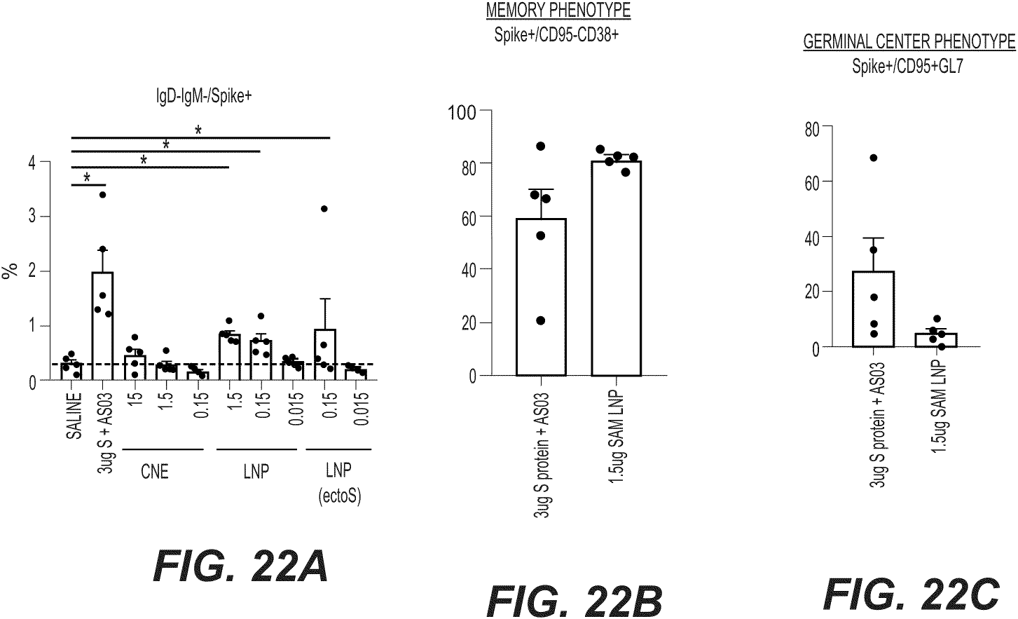

High frequencies of the Spike-specific IgD-IgM- B-cells were observed in mice receiving 1.5 µg and 0.15 µg of the SARS-CoV- 2 Spike_FL-2P SAM (LNP) as well as in mice receiving AS03-adjuvanted recombinant Spike protein (FIG. 22A). Further analysis showed that most of the Spike-specific B-cells from the 1.5 µg SARS-CoV-2 Spike_FL-2P SAM (LNP) and the AS03-adjuvanted recombinant Spike protein groups had a memory phenotype (FIG. 22B, FIG. 22C).

### B-Cell Responses in the Inguinal Draining Lymph Node at 2wp2

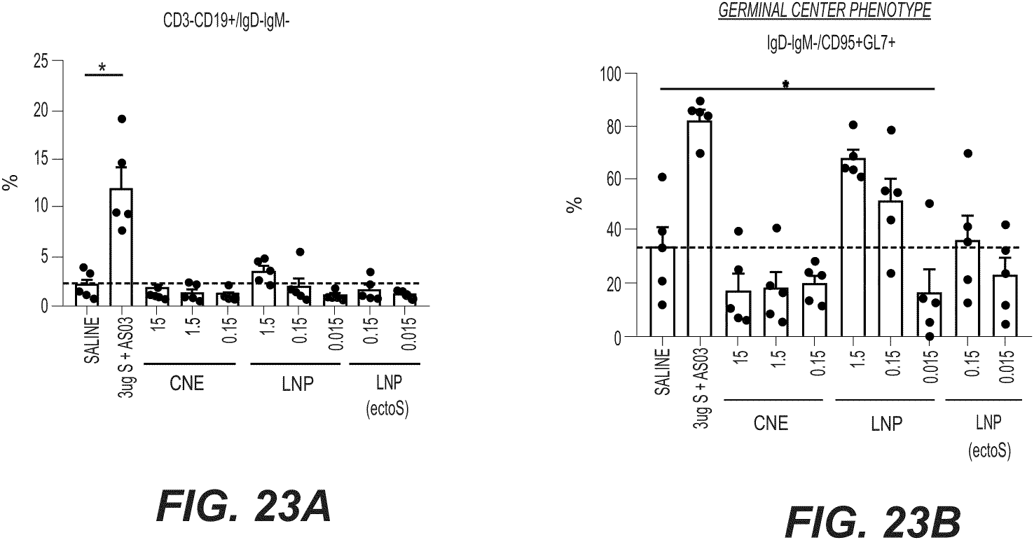

B-cell responses in the pair of inguinal draining lymph nodes were characterized 2 weeks after the second immunization. Frequencies of IgM-IgD- B cells increased in the draining lymph node of mice receiving 1.5 µg SARS-CoV-2 Spike_FL-2P SAM (LNP) and AS03-adjuvanted Spike recombinant protein, and over 70- and 80%, respectively, showed a germinal center phenotype (FIG. 23A, FIG. 23B).

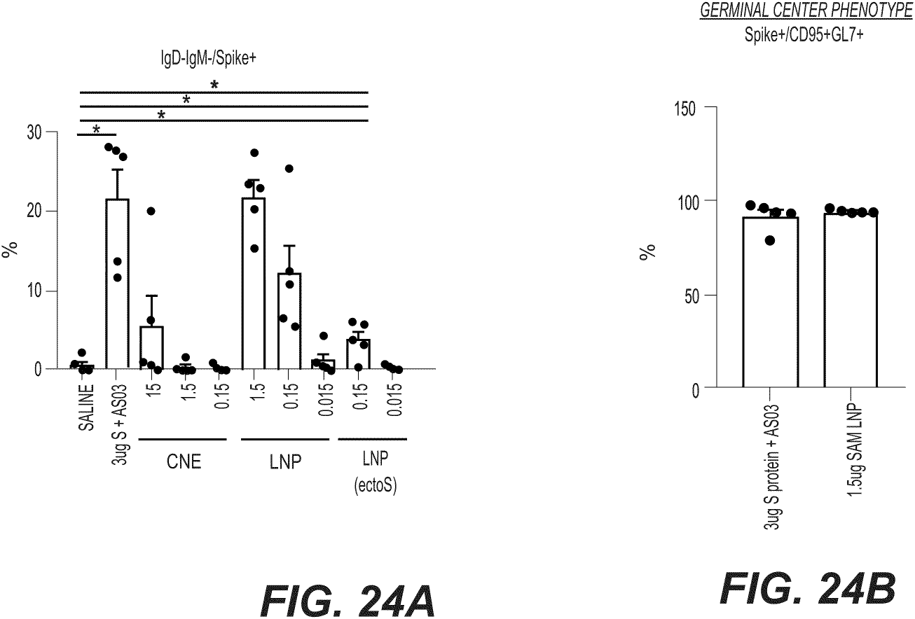

Mice immunized with 1.5 µg and 0.15 µg SARS-CoV-2 Spike_FL-2P SAM (LNP) and AS03-adjuvanted Spike recombinant protein showed Spike-specific IgMIgD- B-cells, with a dose-dependent response observed in the SAM (LNP) groups (FIG. 24A); over 90% of those cells had a germinal center phenotype (FIG. 24B).

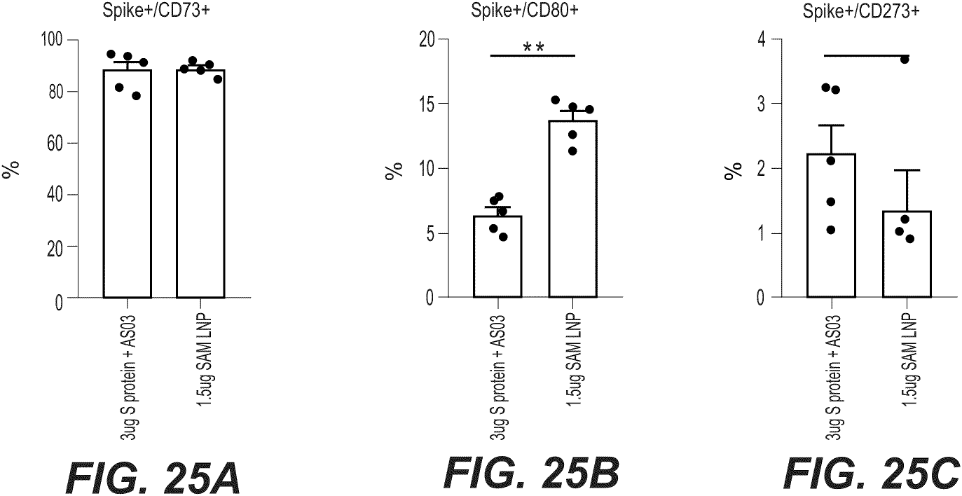

Spike-specific IgM-IgD- B-cells were characterized for the surface expression of CD73, CD80 and CD273 germinal center markers in mice receiving SARS-CoV-2 Spike_FL-2P SAM (LNP) and AS03-adjuvanted Spike recombinant protein (FIG. 25A, FIG. 25B, FIG. 25C). Nearly 90% of the Spike-specific IgM-IgD- B-cells expressed CD73 in both groups; however, the 1.5 µg SARS-CoV-2 Spike_FL-2P SAM group had a trend toward higher frequencies of CD80+ cells and lower frequencies of CD273+ cells compared to the AS03-adjuvanted Spike protein group, suggesting for potentially improved Tfh development, germinal center B-cell survival, and plasma cell generation.

The results showed that high frequency of Spike-specific B-cells was observed in mice receiving the AS03-adjuvanted or the SARS-CoV-2 Spike_FL-2P SAM (LNP) vaccine. The responses observed in the SAM (CNE) and in the SAM-ecto (LNP) groups were overall lower. The highest responses were observed in the inguinal draining lymph node where most Spike specific B-cells have a germinal center phenotype. AS03-adjuvanted Spike protein, 1.5 µg and 0.15 µg SARS-CoV-2 Spike_FL-2P SAM (LNP) vaccinations induced high frequency of IgM-IgD- Spike-specific B-cells, with a dose-dependent response was observed in the SAM LNP groups. Overall, those data showed a strong germinal center response for the 1.5 µg SARS-CoV-2 Spike_FL-2P SAM (LNP) vaccine, indicating B-cell responses of high quality and quantity with a potential induction of high-affinity long lasting B cells.

### T-Cell-Mediated Immunity - Spike-Specific T-Cell-Mediated Immunity (Spike CMI)

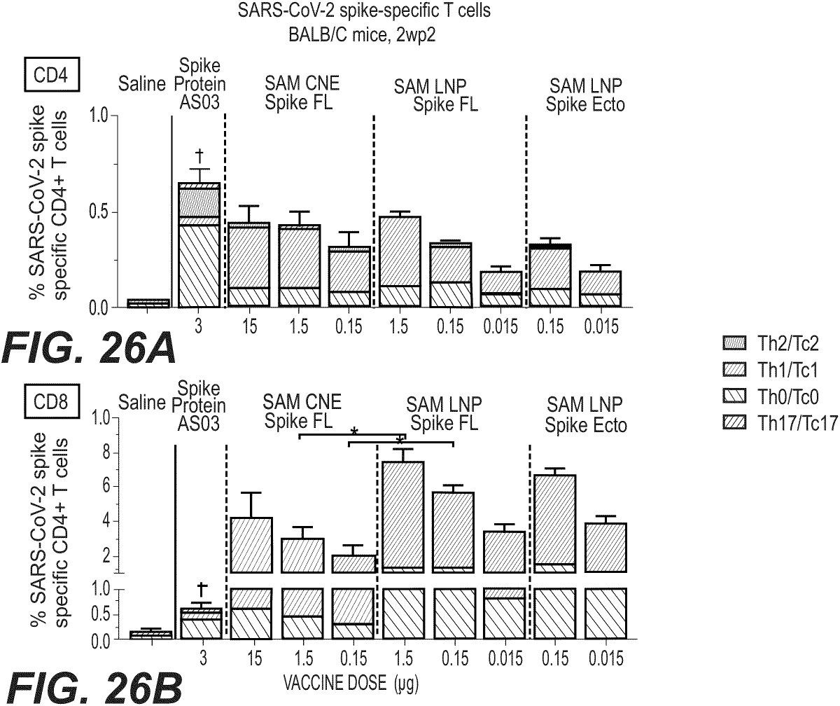

To assess the T-cell-mediated immune (CMI) responses induced by each vaccine candidate, spleens were collected from 5 mice/group at 2wp2 (Day 36) and SARS-CoV-2 Spike-specific T-cell responses were measured using intracellular cytokine staining (ICS) and multiparametric flow cytometry for individual mice. The analysis of cell-mediated immune responses included measurement of magnitude of total spike-specific CD4+ and CD8+ T-cells and magnitude of various T helper (Th) subsets within the total spike- specific CD4+ (denoted as Th0, Th1, Th2, and Th17) and CD8+ (denoted as Tc0, Tc1, Tc2, and Tc17) T cells (FIG. 26A, FIG. 26B).

CoV-2 Spike SAM (LNP) vaccines induced significantly higher levels of total spike-specific CD8+ T-cells than CoV-2 Spike SAM (CNE) vaccines at comparable doses (p<0.0001 with 13.7-fold change of the 1.5 µg Spike_FL-2P SAM (LNP) group over the AS03-adjuvanted group). No significant difference was observed in frequencies of total spike-specific CD8+ T-cells between FL spike vs. ecto CoV-2 Spike SAM (LNP) vaccines at comparable doses. All CoV-2 Spike SAM vaccines induced significantly higher total spike-specific CD8+ T-cells than CoV-2 Spike protein adjuvanted with AS03. All CoV-2 Spike SAM groups had significantly higher Spike-specific CD4+ Th1 responses than CoV-2 Spike protein adjuvanted with AS03. (p<0.0001; 8-fold changes of the 1.5 µg Spike_FL-2P SAM (LNP) group over the AS03-adjuvanted group). All CoV-2 Spike SAM candidate vaccines induced significantly lower frequencies of CD4+ T cells than AS03-adjuvanted CoV-2 Spike protein vaccine, apart from the SAM (CNE) 15 µg and 1.5 µg doses and the SAM (LNP) 1.5 µg dose groups. CoV-2 Spike protein vaccine adjuvanted with AS03 elicited primarily Th0 and Th2 responses from the CD4+ T-cell compartment, with significantly higher Th0, Th2, and Th17 responses than SAM vaccines. A dose response was observed for all SARS-CoV-2 Spike SAM vaccines from primarily the Th0, Th1 subsets.

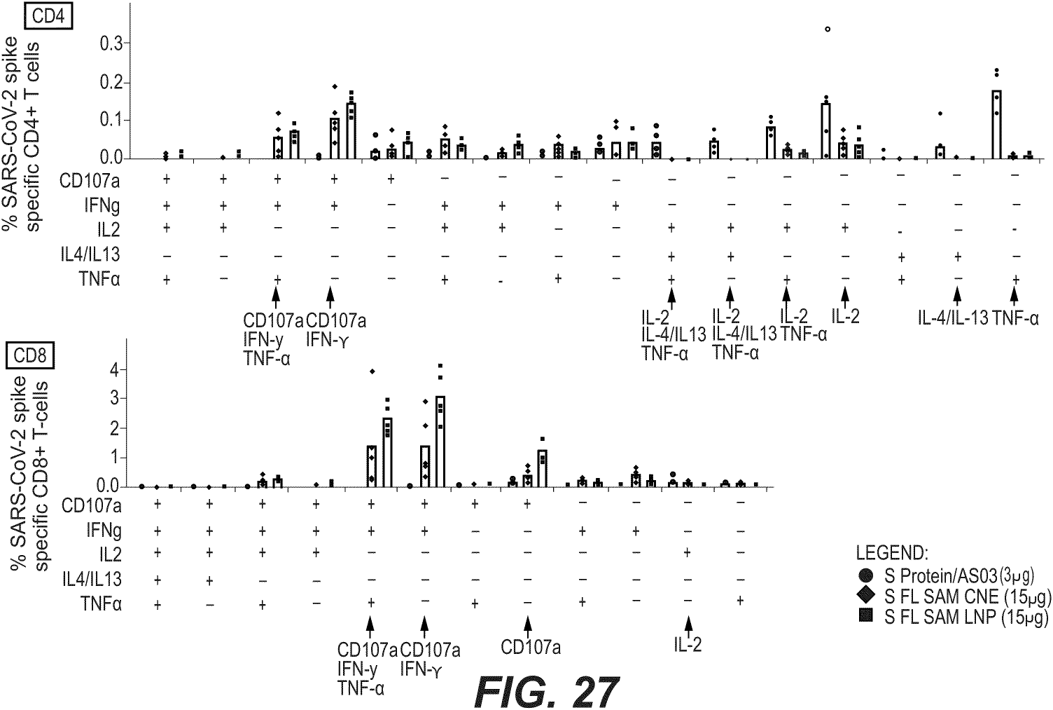

Further characterization of the spike-specific polyfunctional (FIG. 27) CD4+ and CD8+ T-cell cytokines responses was also performed. CoV-2 Spike SAM (LNP) and CoV-2 Spike SAM (CNE) vaccine groups induced primarily CD107a+ IFN-γ+ TNF-α+ triple positives and CD107a+ IFN-γ+ double positives in CD4+ T-cells. CoV-2 Spike protein adjuvanted with AS03 vaccine group induced primarily IL-2 and TNF-α single positives, and IL-2+ TNF-α+ double positives at nominal levels in CD4+ T-cells. SARS-CoV-2 Spike SAM (LNP) and SARS-CoV-2 Spike SAM (CNE) vaccines induced primarily CD107a+ IFN-γ+ TNF-α+ triple positives and CD107a+ IFN-γ+ double positives in CD8+ T-cells. The distribution profile of the CD8+ T-cell polyfunctional response indicates robust vaccine-induced cytolytic, immunomodulatory, pro-inflammatory, and potential anti-viral activity and is promising for the SAM platform. SARS-CoV-2 Spike protein/AS03 vaccine group induced primarily low levels of CD107a+ (teal) and IL-2+ single positives in CD8+ T-cells.

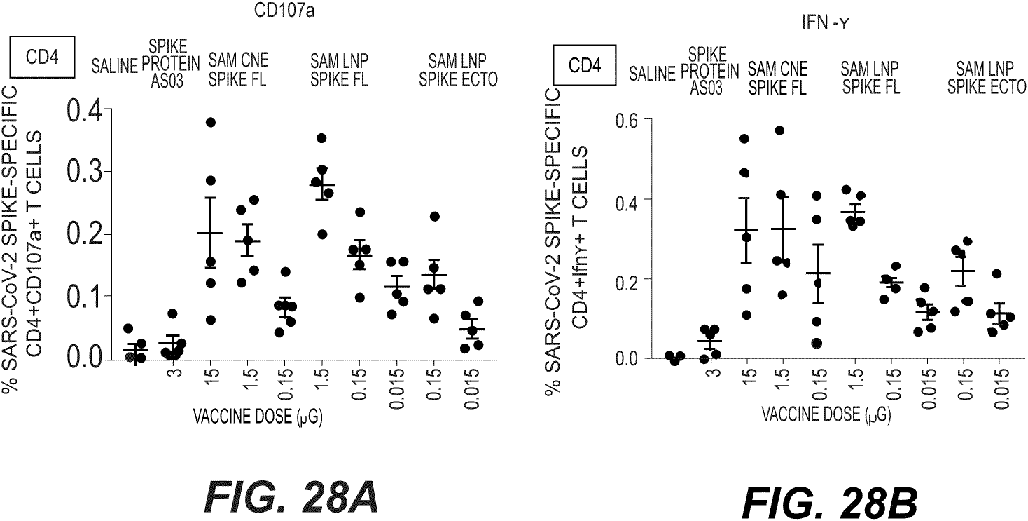

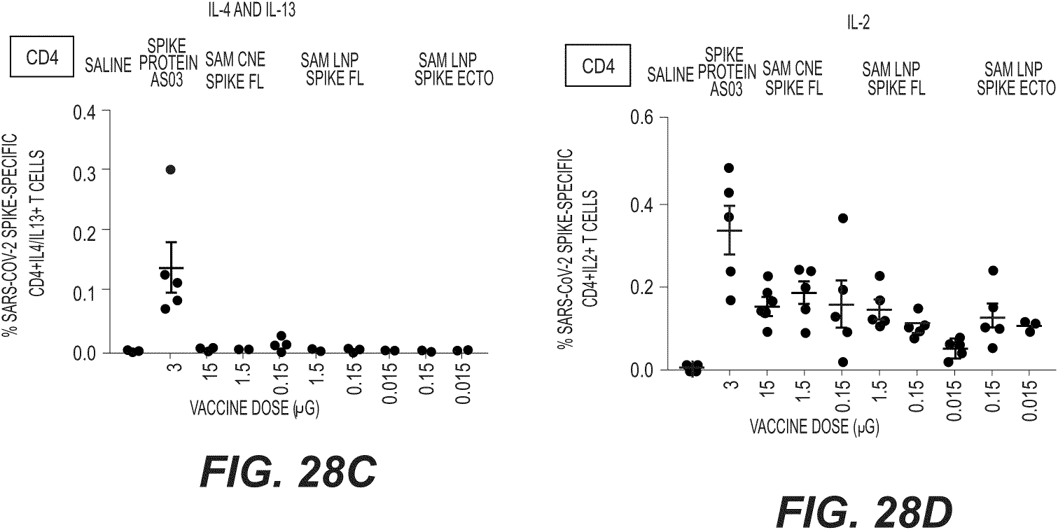

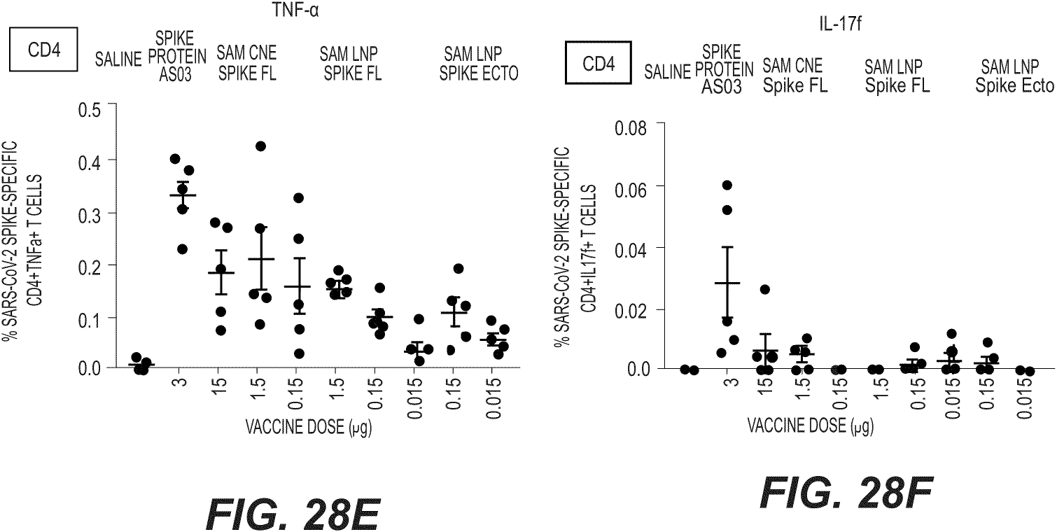

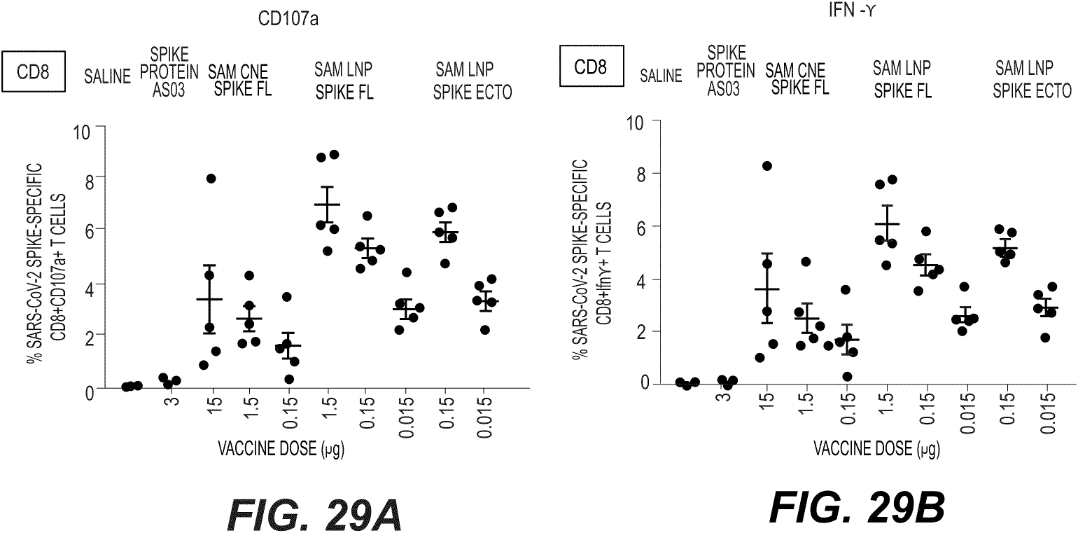

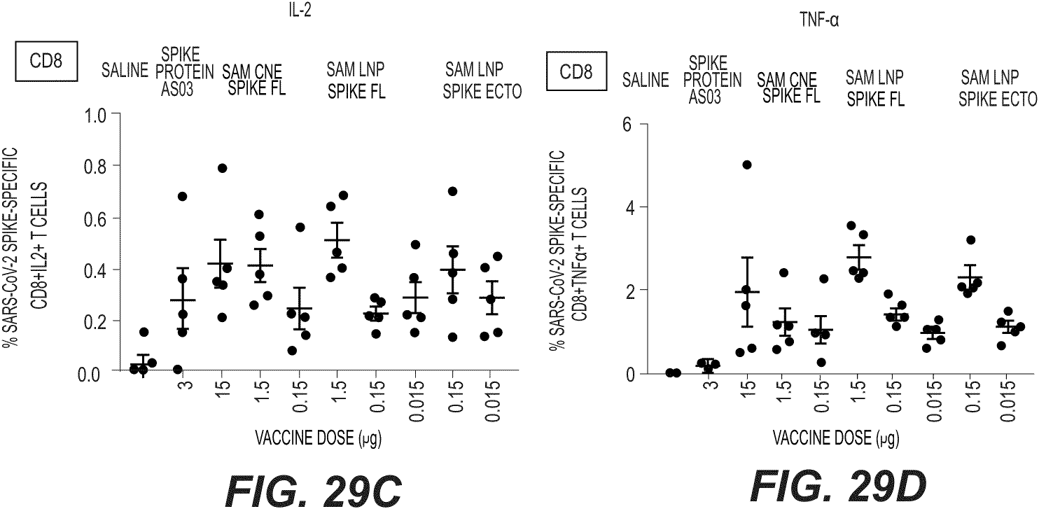

Further characterization of the spike-specific individual (FIG. 28A, FIG. 28B, FIG. 28C, FIG. 28D, FIG. 28E, FIG. 28F and FIG. 29A, FIG. 29B, FIG. 29C, FIG. 29D) CD4+ and CD8+ T-cell cytokines responses was also performed. With regard to CD4+, all SARS-CoV-2 Spike SAM vaccine groups induced significantly higher CD107a and IFN-γ than AS03-adjuvanted SARS-CoV-2 Spike protein vaccine from CD4+ T-cells. The SARS-CoV-2 Spike protein/AS03 vaccine induced significantly higher IL-4/IL-13, IL-2, TNF-α and IL-17F than all doses of SAM vaccines from CD4+ T cells. With regard to CD8+, all SARS-CoV-2 Spike SAM vaccine groups induced significantly higher levels of CD107a, IFN-γ, IL-2, and TNF-α than SARS-CoV-2 Spike protein/AS03 vaccine from the CD8+ T-cell compartment. SARS-CoV-2 Spike SAM (LNP) vaccines induced significantly higher CD107a and IFN-γ than same dose of SARS-CoV-2 Spike SAM (CNE) in CD8+ T-cells. A statistically significant dose response was observed for SARS-CoV-2 Spike SAM (LNP) vaccines for CD107a, IFN-γ, and TNF-α from the CD8+ T-cells.

### T-Cell-Mediated Immunity - Non-Structural Proteins (nsP) Specific Cell-Mediated Immunity (nsP CMI)

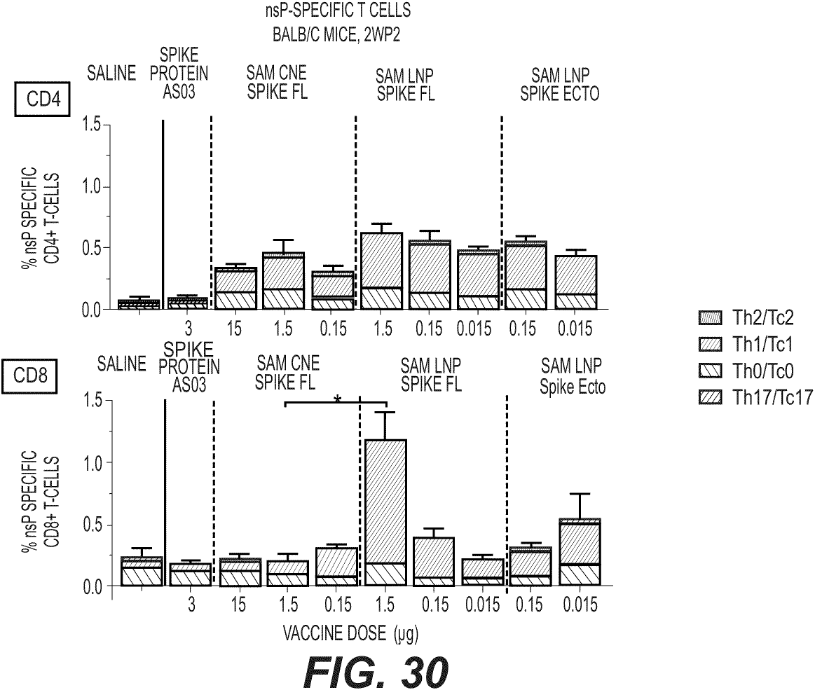

The cell-mediated immune responses to SAM nsPs were assessed in spleens at 2wp2 (Day 36). The analysis included measurement of magnitude of total SAM nsP-specific CD4+ and CD8+ T-cells using combination of nsP-1, nsP-2, nsP-3, and nsP-4 peptide pools (FIG. 30).

All SARS-CoV-2 Spike SAM vaccines induced low levels of anti-nsP immunity from the Th0 and Th1 subsets within both CD4+ and CD8+ T-cell compartments. SARS-CoV-2 Spike_FL-2P SAM (LNP) vaccine at 1.5 µg dose induced significantly higher frequencies of nsP-specific T-cells than comparable dose of SARS-CoV-2 Spike_FL-2P SAM (CNE). A dose response was observed for nsP-specific T-cells for SARS-CoV-2 Spike_FL-2P SAM (LNP) vaccine, which was significant for the Th0 subset in CD4+ T cells and significant for Th0, Th1 and total CD8+ T-cells. Background frequencies of nsP-specific CD4+ and CD8+ T-cells were observed in the saline and AS03-adjuvanted SARS-CoV-2 Spike protein.

### T-Cell-Mediated Immunity - T Follicular Helper (Tfh) Responses in the Spleen

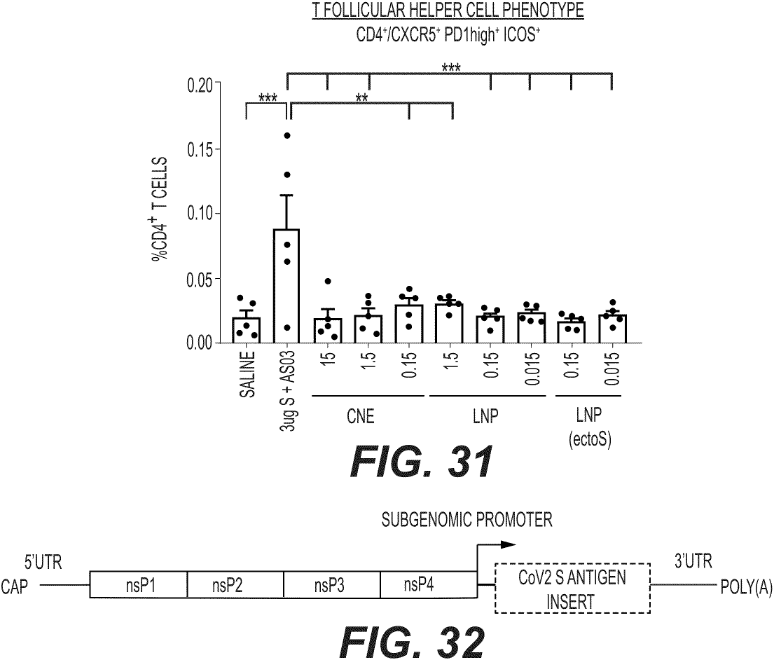

The total Tfh responses were measured in spleen at 2wp2 (Day 36). Vaccination with AS03-adjuvanted SARS-CoV-2 Spike protein induced significantly higher frequencies of Tfh cells compared to the saline and SAM (CNE) and SAM (LNP) vaccinated groups in the spleen (FIG. 31).

### Example 11. Making and Testing SAM Having Derivatives of Constructs A and B Containing Putative Expression and Secretion Modifications

In most constructs, the 16 or 18 N-terminal residues (putative antigen signal sequence) were replaced by heterologous signal sequences. In some constructs, the ER retention signal was replaced. See Tables 2 and 3, above. The constructs were evaluated in mammalian cells following electroporation of SAM RNA into BHK cells using antigen expression by flow cytometry, immunoblots and immunofluorescence assays, to investigate protein in cell lysates, on the cell surface and on cell supernatant.

### 11.1 Full Length SARS-COV2 S Constructs

Table 15: SARS-CoV-2 Spike SAM mutants. In this Table and related text, a shorthand naming convention is adopted, referring to “pKL026” of Table 3 as “KL26”. In this section 11.1, “WT” refers to the pJW19 construct, which encodes spike protein having its native signal sequence.

| | |
|---|---|
| KL26 | c-terminal deletion (after 1273aa) in pJW19 |
| KL28 | Replacement of the first 18aa with GauLUCss in pJW19 |
| KL31 | Replacement of the first 16aa with CD33ss in pJW19 |
| KL33 | Replacement of the first 16aa with hlL2ss in pJW19 |
| KL34 | Replacement of the first 18aa with hlL2ss in pJW19 |
| KL35 | Replacement of the first 16aa with hlgEss in pJW19 |
| KL36 | Replacement of the first 18aa with hlgEss in pJW19 |
| KL37 | Replacement of the first 16aa with hLCkappass in pJW19 |
| KL38 | Replacement of the first 18aa with hLCkappass in pJW19 |
| KL40 | Replacement of the first 18aa with JEVSss in pJW19 |
| KL42 | Replacement of the first 18aa with JEVLss in pJW19 |
| KL43 | Replacement of the first 16aa with mLCkappass in pJW19 |
| KL44 | Replacement of the first 18aa with mLCkappass in pJW19 |
| KL47 | Replacement of the first 16aa with GauLUCss (AKP) in pJW19 |
| KL48 | Replacement of the first 18aa with GauLUCss (AKP) in pJW19 |

In this regard, BHK-21 cells (passage 27) were placed in T225 flask at approximately 1e⁷ cells/flask and culture overnight. SAM RNA was diluted to 50 ng/µl.

TABLE 16

Electroporation of RNA

| SAM | ng/µl | Use µl | H₂O µl | Actual ng/µl | µl for 250 ng |
|---|---|---|---|---|---|
| JW19 | 1700 | 2 | 66.00 | 52.92 | 4.72 |
| KL26 | 424.4 | 4 | 29.95 | 48.89 | 5.11 |
| KL28 | 770 | 2 | 28.80 | 65.27 | 3.83 |
| KL31 | 1153 | 2 | 44.12 | 57.64 | 4.34 |
| KL33 | 452 | 4 | 32.16 | 43.97 | 5.69 |
| KL34 | 660.2 | 2 | 24.41 | 44.78 | 5.58 |
| KL35 | 478 | 4 | 34.24 | 47.94 | 5.21 |
| KL36 | 600.5 | 2 | 22.02 | 40.22 | 6.22 |
| KL37 | 570.2 | 2 | 20.81 | 44.08 | 5.67 |
| KL38 | 729.1 | 2 | 27.16 | 41.91 | 5.97 |
| KL40 | 591.6 | 2 | 21.66 | 38.32 | 6.52 |
| KL42 | 643.4 | 2 | 23.74 | 51.1 | 4.89 |
| KL43 | 1406 | 2 | 54.24 | 47.1 | 5.31 |
| KL44 | 1169 | 2 | 44.76 | 45.39 | 5.51 |
| KL47 | 1512 | 2 | 58.48 | 48.3 | 5.18 |
| KL48 | 769.9 | 2 | 28.80 | 43.11 | 5.80 |
| GFP (A848) | 180 | 4 | 10.40 | 45.47 | 5.50 |

### SAM Was Mixed With Mouse Thymus RNA (Mock):

TABLE 17

SAM Mix

| | | |
|---|---|---|
| | Per electroporation | 2+ 0.5 electroporations |
| BHK-21 cells | 250 µl of 4e⁶ cells/ml | 625 µl |
| RNA (total 4.2 µg) | 100 ng SAM | 250 ng |
| | Mouse thymus (Mock) RNA 4.1 µg | 10.25 µg |

Cells were Trypsinized and collected, wash with cold Opti-MEM once, resuspend cells in cold Opti-MEM at 4e⁶ cells/ml. 6-well plates were prepared with 2 ml medium-1 % FBS/well. Plates were pre-warmed in incubator. Cells were mixed with RNA and electroporate cells/RNA mixture with Bio-Rad Gene Pulser: (Voltage 120 V, Pulse length 25 ms, Pulse 1. Pulse interval 0, Cuvette 2 mm). 250 µl medium from the pre-warmed 6-well plate were added, resuspend and transfer cells back to 6-well plate. At 18 hpt, supernatant were collected and process cells for flow cytometry and Western blot (lysis). GFP SAM were used as positive control for RNA electroporation.

### Flow Cytometry

Supernatant was collect and saved at -80° C. Added 200 µl 0.25% trypsin-EDTA to cell monolayer and incubated at 37° C. for 5 min. 500 µl medium was added, cells were resuspended and transferred 150 µl cells/well to 96-well U-bottom plates and 150 µl cells to a tube for lysis.

Spike surface staining: Cells were spun at 1200 rpm for 5 min and medium discarded. Cells were wash with 150 µl cold PBS-2.5% FBS. Cells were spun 1200 rpm 5 min, buffer discarded, and cells kept on ice. Cells were resuspended with 100 µl 1:1000 diluted mouse anti-SARS-CoV-2 S mAb in PBS-2.5% FBS and incubated on ice for 1 hr. Cells were spun 1200 rpm 5 min, washed with 150 ul PBS-2.5% FBS, spun 1200 rpm 5 min, buffer discarded and cells kept on ice. Cells were resuspended with 100 µl 1:1000 diluted goat anti-mouse IgG Alexa 488 in PBS-2.5% FBS and incubated on ice for 30 min and spun 1200 rpm 5 min. Cells were wash with 150 µl PBS-2.5% FBS, spun 1200 rpm 5 min, buffer discarded, and cells kept on ice. Cells were resuspend with 150 µl PBS-0.25% BSA. Flow cytometry was ran on MACSQuant VYB with channel B1 (GFP/488).

The following steps, with fix/permeabilization, were for SARS-CoV-2 Spike whole cell staining: Cells were spun 1200 rpm 5 min and medium discarded. Cells were resuspended with 100 µl Fix/Perm buffer, incubated at 4° C. for 20 min, and spun 1200 rpm 5 min. Cells were wash with 150 µ perm buffer once, spun 1200 rpm for 5 min and buffer discarded.

(Spike staining) Cells were mixed cells with 100 µl 1:1000 diluted anti-SARS-CoV-2 Spike mAb in Perm buffer, incubated at room temperature for 1 hr., and spun 1200 rpm 5 min. Cells were wash with 150 µl Perm buffer once, spun 1200 rpm 5 min and buffer discarded. Cells were mixed with 100 µl 1:1000 goat anti-mouse IgG Alexa 488, incubated at room temperature for 1 hr., spun 1200 rpm 5 min, and buffer discarded. Cells were wash with 150 µl Perm buffer once, spun 1200 rpm 5 min and buffer discarded. Cells were resuspend in 150 µl PBS-BSA (0.25%) and flow cytometry ran on MACSQuant VYB with channel B1 (GFP/488).

### Results

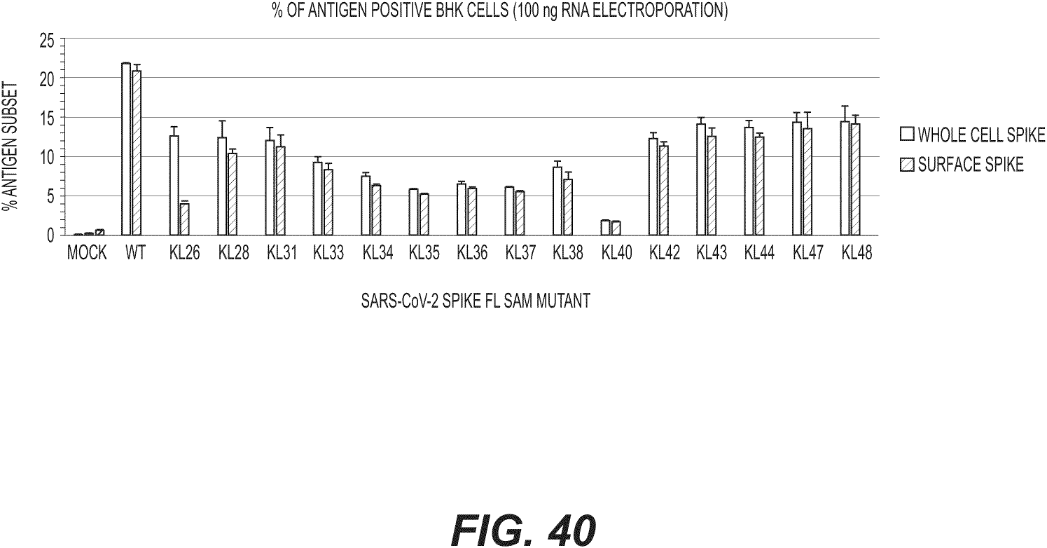

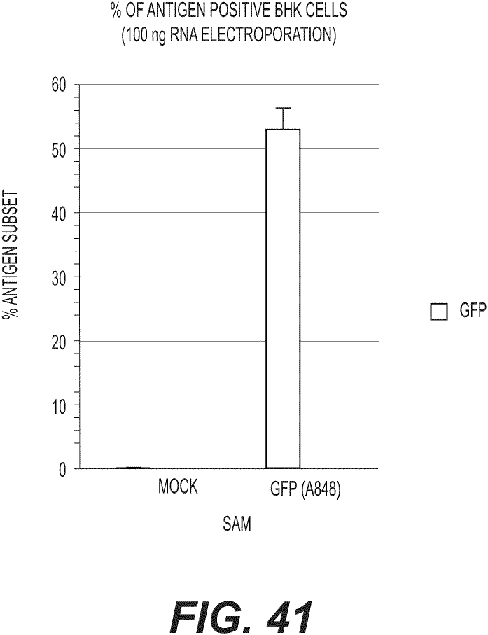

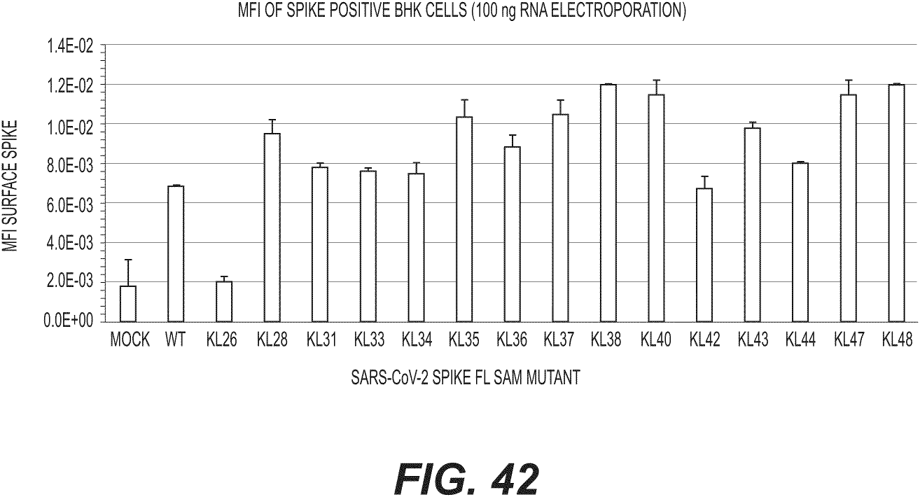

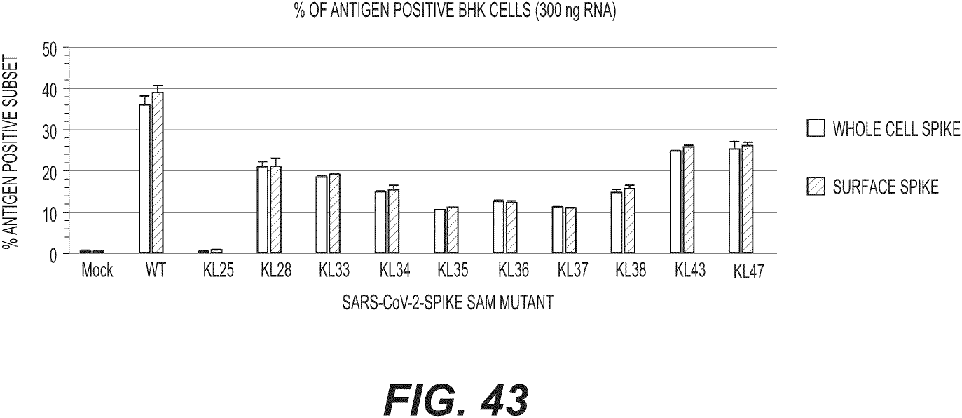

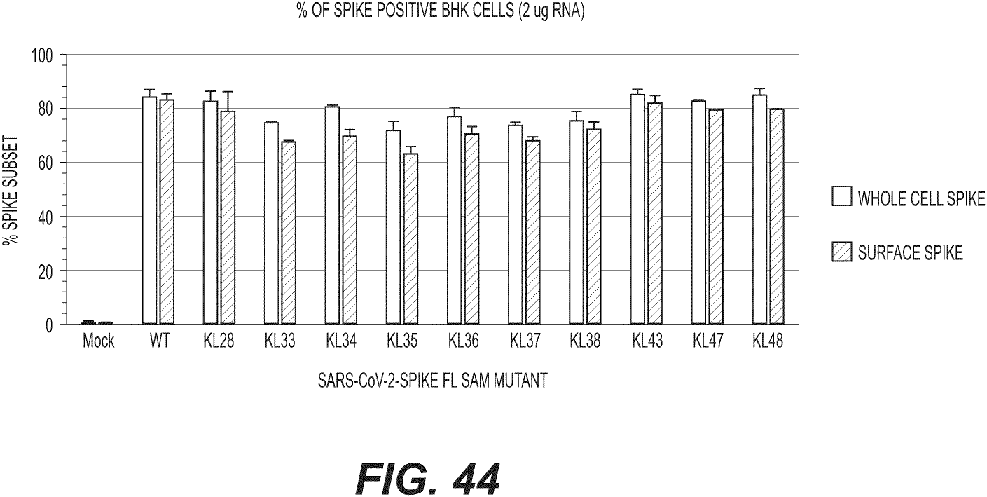

Results of % of antigen positive cells are shown in FIG. 40, FIG. 41. and FIG. 42. Both SAM encoding full-length spike protein having a wild-type signal sequence (WT Spike SAM; JW19) and GFP SAM worked well as positive controls. GFP SAM at 100 ng achieved 53% GFP positive cells. FIG. 41. WT Spike SAM showed 21% spike positive cells by both whole cell and surface spike staining. FIG. 40. The majority of mutants ranged from 5% to 15% spike positive cells (more than half at 10-15% range) by both whole cell and surface Spike staining. KL26 showed fair % of whole cell spike staining, but low % and MFI of surface staining, indicating low surface expression. KL40 showed very low % of antigen positive cells by all staining (whole cell and surface spike staining). Based on > 10% spike positive cells and > 1.2 fold of WT surface spike MFI as cut offs, KL28, KL43, KL47, and KL48 are noted. 8. Comparing surface spike histograms of mutants against WT, KL48 had a clear shift to higher surface spike level than WT. FIG. 43 and FIG. 44 depicts % of spike positive BHK cells with 300 ng and 2 µg RNA electroporation, respectively. Due to low % of spike positive cells, KL35, 36, 37 and 38 remain unclear and require more studies.

### 11.2 SARS-CoV-2 Spike Ecto SAM Mutants

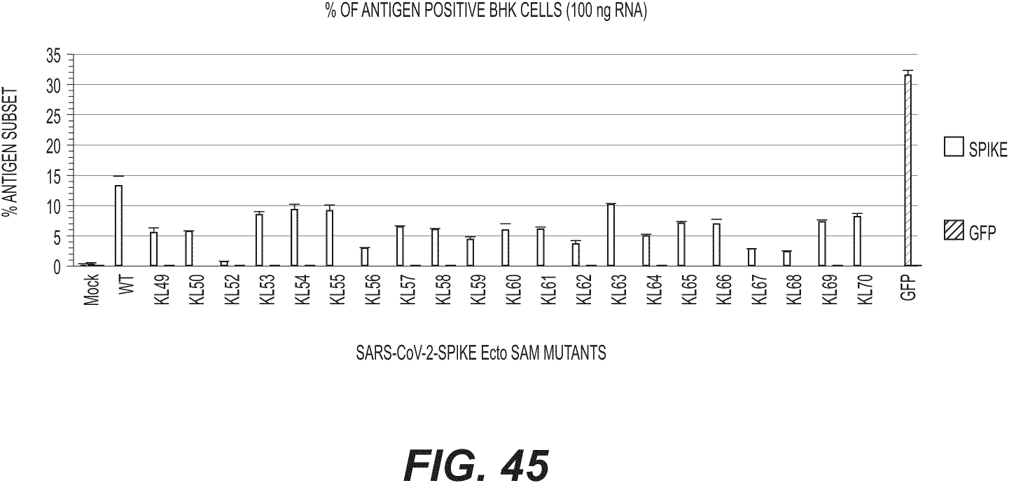

SARS-CoV-2 spike Ecto SAM mutants were prepared as previously described, % of antigen positive BHK cells are shown in FIG. 45. Related SARS-CoV-2 spike Ecto SAM mutants’ supernatant and cell lysates were frozen. In this section 11.2, “WT” refers to the pJW18 construct encoding the ecto spike protein having the native signal sequence.

Concentrate supernatant: Supernatants were thawed and clarified by centrifugation and 2000 rpm for 5 min. 500 µl supernatant was added to Amicon Ultracel-10K, and spun 13.2K rpm for 15 min at 4° C. Flow-through was removed, column put back in reverse position, spun at 13.2 K rpm for 1 min at 4° C., resulting in 50 µl concentrated supernatant.

Western Blot: Cell lysates were thawed where approximately 8.5e⁵ cells were lysed in 112 ul RIPA buffer. Cell lysate or concentrated supernatant were mixed with SDS Sample buffer and DTT, heat on 100° C. heating block for 10 min.

TABLE 18

Western Blot

| | | |
|---|---|---|
| | Supernatant | Cell lysate |
| Cell lysate | | 14 µl (app. 1e⁵ cells) |
| Supernatant concentrated | 21 µl (app. 1e⁵ cells | |
| SDS-Sample buffer (4x) | 7 µl | 6 µl |
| 1 M DTT | 1 µl | 1 µl |

Criterion TGX Precast Gel 4-20% was set up in Criterion Cell apparatus, samples were loaded and run at constant amp of 60 mA/gel (120 mA for 2 gels). Proteins were transferred from gel to NC membrane on iBlot2 apparatus with P0 program (20 V for 1 min, 23 V for 4 min, 25 V for 2 min). Protein-free (PBS) Blocking Buffer was used to block NC membrane for 1 hr. The membrane was incubated with 1:4000 diluted mouse anti-SARS-CoV-2 spike mAb and rabbit anti-Actin mAb in blocking buffer overnight then washed with PBS-0.1% Tween 20 (PBST) three times. Membrane was incubated with secondary antibodies 1:20,000 diluted in blocking buffer for 1 hr. Membrane was wash with PBST three times and infrared (IR) image was captured on Odyssey CLx with 700 nm and 800 nm channels.

### Results

Mouse anti-spike mAb detected doublet high MW (>260 kDa) spike protein bands in supernatants of S Ecto SAM-transfected BHK cells. The mAb-detected protein bands seemed to be spike-specific, not seen with Mock RNA. The levels of spike proteins in supernatant of KL70 (corresponding to FL mutant KL48) were higher than WT. The levels of spike proteins in supernatants of other mutants were similar or lower than WT.

Mouse anti-spike mAb detected a single 180-kDa spike protein band in cell lysate of S Ecto SAM-transfected BHK cells. The levels of spike proteins in cell lysates correlated with % of spike protein positive cells. All mutants were lower than WT. Overall, KL70 showed lower spike protein in cell lysate but higher spike protein in supernatant than WT, suggesting its mutation increased secretion of spike Ecto.

Based on the amount of spike protein in supernatant and cell, the supernatant-to-cell ratio of most mutants were within 1-2 fold of WT, except for KL49, KL50, KL69 and KL70. (text, a shorthand naming convention is adopted, referring to “pKL049” of Table 2 as “KL49”.) The supernatant to cell ratios of mutant KL49, KL50, KL69 and KL70 were higher than 2-fold of WT. KL70 was the highest, being 6-fold of WT. The supernatant to cell ratio was also calculated by using Actin-normalized Spike signal. Based on Actin-normalized spike signal in supernatant and cell, the supernatant to cell ratio of most mutants were within 1-3 fold of WT, except for KL50, KL69 and KL70. The actin-normalized supernatant to cell ratios of mutant KL50, KL69 and KL70 were higher than 3-fold. KL70 was the highest, being 8-fold of WT(wild type). Overall, KL49, KL50, KL69 and KL70 showed higher supernatant to cell ratio of spike protein, among them KL70 was the highest.

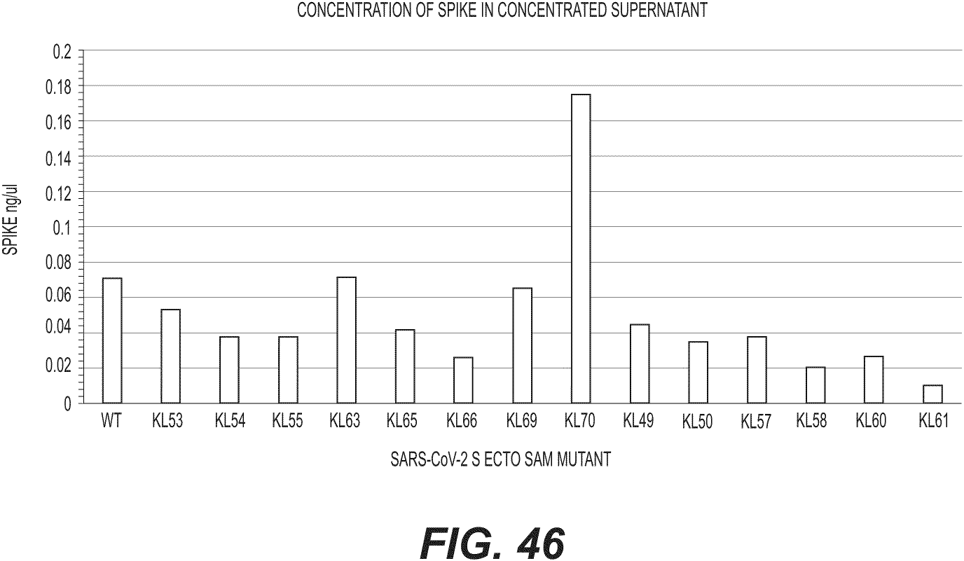

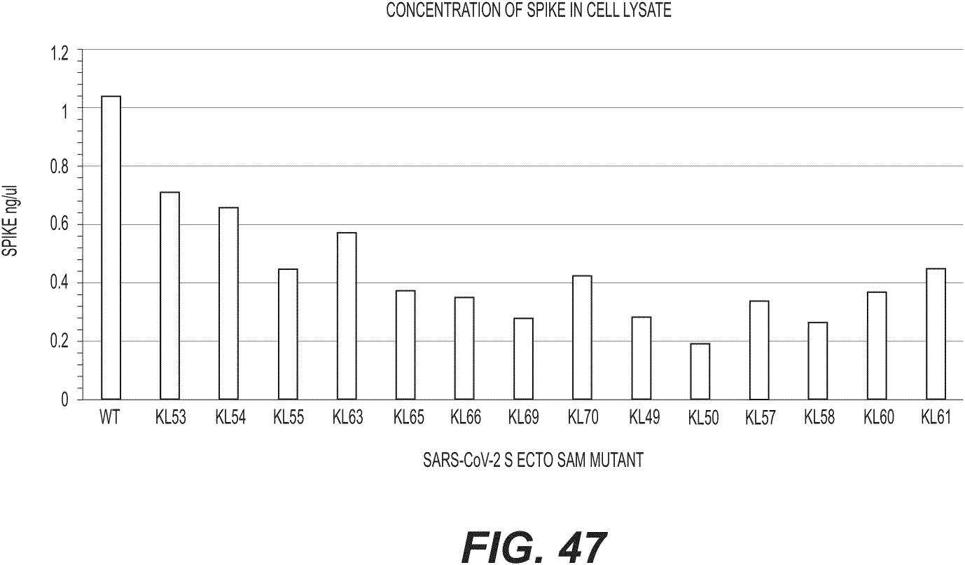

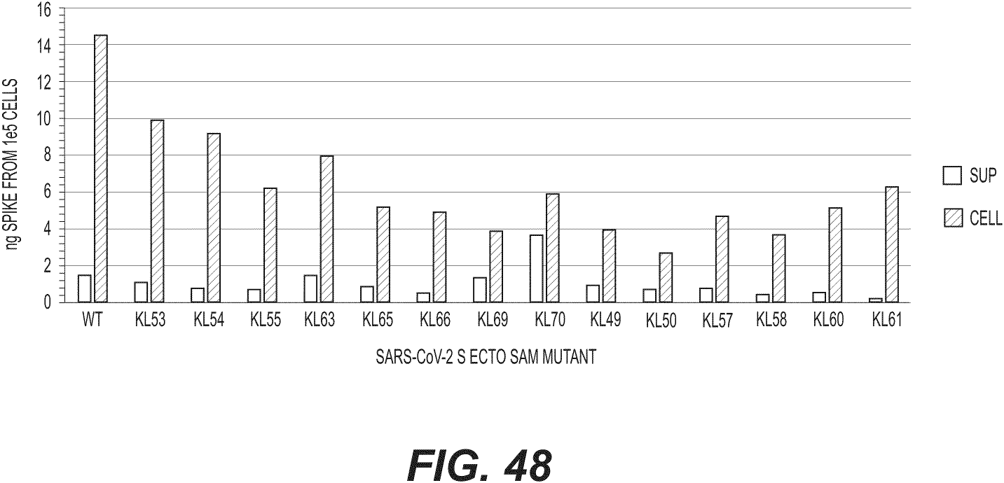

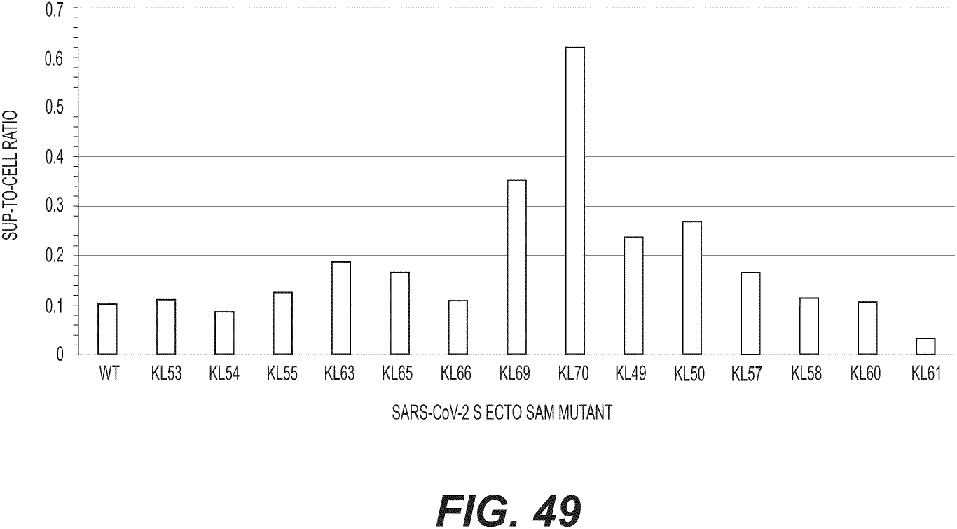

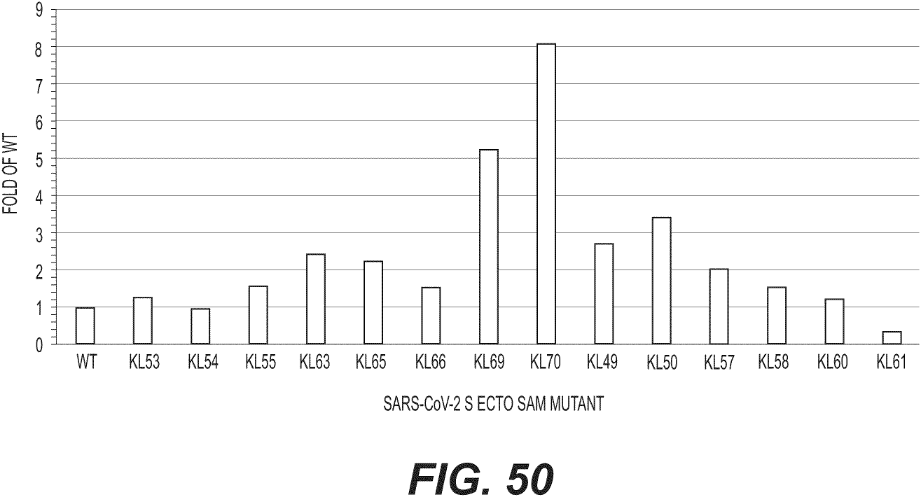

Concentration of spike protein in concentrated supernatant is noted in FIG. 46. Concentration of spike protein in cell lysate is illustrated in FIG. 47. Concentration of spike protein in cell lysate and supernatant is illustrated in FIG. 48. Supernatant-to-cell ratio of spike protein is illustrated in FIG. 49. Supernatant-to-cell ratio of spike protein normalized to actin and wild type is illustrated in FIG. 49.

### Summary of Example 11

A panel of SAM encoding antigen mutants, in both FL and Ecto forms as described above, with alternative signal sequences replacing the first 16 or 18 aa of the antigen, were generated. The FL antigens retained the membrane spanning region of the antigen and thus, if expressed correctly, would demonstrate surface expression. The Ecto forms lacked the membrane spanning region and thus, if expressed correctly, would be secreted. Screening of FL mutants by flow cytometry identified 5 mutants that show increased surface antigen expression: 1 having Gaussia luciferase (GLuc); 2 having Gaussia luciferase-AKP (GLuc-AKP); 2 having IgG light chain kappa (LCk). Screening of Ecto mutants by Western blot identified 3 mutants with enhanced secretion of antigen: 2 having Gaussia luciferase-AKP (GLuc-AKP); 1 having Gaussia luciferase (GLuc). Overall, the GLuc signal sequence, especially GLuc-AKP, was identified as strongly enhancing surface expression/secretion.

We claim:

1. A self-replicating ribonucleic acid (RNA) comprising a first sequence or a second sequence, the first sequence: 1) being at least 95% identical to SEQ ID NO: 122 and the same length as, or up to 30 nucleotides shorter than, SEQ ID NO: 122 and 2) encoding a first amino acid sequence being at least 99% identical to SEQ ID NO:26 and the same length as, or up to 10 amino acids shorter than, SEQ ID NO:26, the second sequence: 3) being at least 95% identical to SEQ ID NO: 145 and the same length as, or up to 30 nucleotides shorter than, SEQ ID NO:145 and 4) encoding a second amino acid sequence being at least 99% identical to SEQ ID NO:49 and the same length as, or up to 10 amino acids shorter than, SEQ ID NO:49.

2. The self-replicating RNA of claim 1, wherein the first sequence or the second sequence further comprises a third sequence, the third sequence encoding a heterologous signal sequence, wherein the heterologous signal sequence comprises a positive charge at its N terminus.

3. The self-replicating RNA of claim 2, wherein the heterologous signal sequence has a sequence selected from:
a) a Gaussian Luciferase signal sequence as shown in SEQ ID NO:2,
b) a human CD5 signal sequence as shown in SEQ ID NO:3,
c) a human CD33 signal sequence as shown in SEQ ID NO:4,
d) a human IL2 signal sequence as shown in SEQ ID NO:5,
e) a human IgE signal sequence as shown in SEQ ID NO:6,
f) a human Light Chain Kappa signal sequence as shown in SEQ ID NO:7,
g) a JEV short signal sequence as shown in SEQ ID NO:8,
h) a JEV long signal sequence as shown in SEQ ID NO:9,
i) a Mouse Light Chain Kappa signal sequence as shown in SEQ ID NO:10,
j) a SSP signal sequence as shown in SEQ ID NO:11,
k) a Gaussian Luciferase (AKP) signal sequence as shown in SEQ ID NO:12, and
1) a variant of any one of sequences a)-k) having 1, 2, 3, 4, or 5 amino acid residue deletions, insertions, or substitutions.

4. The self-replicating RNA of claim 2, wherein the heterologous signal sequence has a sequence selected from:
a) a Gaussian Luciferase signal sequence as shown in SEQ ID NO:2,
b) a human Light Chain Kappa signal sequence as shown in SEQ ID NO:7,
c) a Gaussian Luciferase (AKP) signal sequence as shown in SEQ ID NO:12, and
d) a variant of any one of sequences a)-c) having 1, 2, 3, 4, or 5 amino acid residue deletions, insertions, or substitutions.

5. (canceled)

6. (canceled)

7. The self-replicating RNA of claim 1 comprising from 5′ to 3′ a viral equine encephalitis (VEE) TC-83 replicon a subgenomic promoter, and the first sequence or the second sequence, the VEE TC-83 replicon comprising VEE nonstructural proteins 1-4 (nsP1-4).

8. The self-replicating RNA of claim 7, comprising SEQ ID NO:171,wherein the first sequence or the second sequence is inserted between positions 7561 and 7562 of SEQ ID NO:171.

9. A self-replicating RNA comprising from 5′ to 3′ a sequence comprising: 1) SEQ ID NO:172, 2) a construct encoding: (i) a signal sequence selected from the group consisting of SEQ ID NOS:258-268 and (ii) a first amino acid sequence being at least 99% identical to SEQ ID NO:26 and the same length as, or up to 10 amino acids shorter than, SEQ ID NO:26 or a second amino acid sequence being at least 99% identical to SEQ ID NO:49 and the same length as, or up to 10 amino acids shorter than, SEQ ID NO:49,and 3) a sequence having SEQ ID NO:173.

10. The self-replicating RNA of claim 9, wherein the signal sequence has a sequence selected from the group consisting of SEQ ID NO:258, SEQ ID NO:263, and SEQ ID NO:268.

11. A DNA encoding a self-replicating RNA molecule comprising a first sequence or a second sequence, the first sequence: 1) being at least 95% identical to SEQ ID NO: 122 and the same length as, or up to 30 nucleotides shorter than, SEQ ID NO:122 and 2) encoding a first amino acid sequence being at least 99% identical to SEQ ID NO:26 and the same length as, or up to 10 amino acids shorter than, SEQ ID NO:26, the second sequence: 3) being at least 95% identical to SEQ ID NO:145 and the same length as, or up to 30 nucleotides shorter than, SEQ ID NO:145 and 4) encoding a first amino acid sequence being at least 99% identical to SEQ ID NO:49 and the same length as, or up to 10 amino acids shorter than, SEQ ID NO:49.

12. A composition comprising an immunologically effective amount of the self-replicating RNA of claim 1 and a pharmaceutically acceptable carrier.

13. The composition of claim 12, wherein the pharmaceutically acceptable carrier comprises a biodegradable polymeric microparticle delivery system.

14. The composition of claim 12, wherein the pharmaceutically acceptable carrier comprises a submicron cationic oil-in-water emulsion.

15. The composition claim 12, wherein the pharmaceutically acceptable carrier comprises a liposome.

16. The composition of claim 12 further comprising a nucleic acid sequence that encodes an antigen that differs from the first amino acid sequence or the second amino acid sequence.

17. A method for producing the self-replicating RNA molecule, the method comprising transcribing the DNA of claim 11 to obtain the self-replicating RNA molecule comprising comprising .

18. A method of inducing an immune response against the first amino acid sequence or second amino acid sequence in a subject in need thereof, the method comprising administering to the subject an immunologically effective amount of the self-replicating RNA molecule of claim 1 .

19. (canceled)

20. (canceled)

21. A composition comprising an immunologically effective amount of the DNA of claim 11.

22. A method of inducing an immune response against the first amino acid sequence or second amino acid sequence in a subject in need thereof, the method comprising administering to the subject an immunologically effective amount of the self-replicating RNA molecule of claim 15.
